(19) United States
(12) Patent Application Publication
(10) Pub. No.: US 2007/0026416 A1
(43) Pub. Date: Feb. 1, 2007

(54) Devices and methods for enrichment and alteration of circulating tumor cells and other particles

(76) Inventor: Martin Fuchs, Uxbridge, MA (US)

Correspondence Address: CLARK & ELBING LLP, 101 FEDERAL STREET, BOSTON, MA, 02110, US

(21) Appl. No.: 11/323,961

(22) Filed: Dec. 29, 2005

Related U.S. Application Data
(60) Provisional application No. 60/703,833, filed on Jul. 29, 2005.

(51) Int. Cl. C12Q 1/68; G01N 33/567; C12M 1/34

(52) U.S. Cl. 435/6; 435/7.2; 435/287.2

(57) ABSTRACT

The invention features devices and methods for detecting, enriching, and analyzing circulating tumor cells and other particles. The invention further features methods of diagnosing a condition, e.g., cancer, in a subject by analyzing a cellular sample from the subject.

### CROSS-REFERENCE TO RELATED APPLICATIONS

This application claims the benefit of U.S. Provisional Application No. 60/703,833, filed Jul. 29, 2005, which is hereby incorporated by reference.

### BACKGROUND OF THE INVENTION

The invention relates to the fields of medical diagnostics and microfluidics.

Cancer is a disease marked by the uncontrolled proliferation of abnormal cells. In normal tissue, cells divide and organize within the tissue in response to signals from surrounding cells. Cancer cells do not respond in the same way to these signals, causing them to proliferate and, in many organs, form a tumor. As the growth of a tumor continues, genetic alterations may accumulate, manifesting as a more aggressive growth phenotype of the cancer cells. If left untreated, metastasis, the spread of cancer cells to distant areas of the body by way of the lymph system or bloodstream, may ensue. Metastasis results in the formation of secondary tumors at multiple sites, damaging healthy tissue. Most cancer death is caused by such secondary tumors.

Despite decades of advances in cancer diagnosis and therapy, many cancers continue to go undetected until late in their development. As one example, most early-stage lung cancers are asymptomatic and are not detected in time for curative treatment, resulting in an overall five-year survival rate for patients with lung cancer of less than 15%. However, in those instances in which lung cancer is detected and treated at an early stage, the prognosis is much more favorable.

Therefore, there exists a need to develop new methods for detecting cancer at earlier stages in the development of the disease.

### SUMMARY OF THE INVENTION

The invention features a device for processing a cellular sample. The device includes a channel including a first array of obstacles that form a network of gaps, preferably at least 40 microns, wherein the obstacles are configured to cause one or more first cells to preferentially make contact with the obstacles, and wherein at least some of the obstacles include one or more binding moieties that selectively bind the first cells. The device can be configured such that fluid flows through the gaps such that the fluid is divided unequally into a major flux and a minor flux. The first cells can be epithelial cells, cancer cells, bone marrow cells, fetal cells, progenitor cells, stem cells, foam cells, mesenchymal cells, immune system cells, endothelial cells, endometrial cells, connective tissue cells, trophoblasts, bacteria, fungi, or pathogens.

The cellular sample can be blood, sweat, tears, ear flow, sputum, lymph, bone marrow suspension, urine, saliva, semen, vaginal flow, cerebrospinal fluid, brain fluid, ascites, milk, secretions of the respiratory, intestinal, or genitourinary tract, amniotic fluid, or a water sample.

The array of obstacles can be a two-dimensional array and can include a plurality of rows, each successive row being offset by less than half of the period of the previous row. The array of obstacles can include a staggered two-dimensional array of obstacles, and can further include a second staggered two-dimensional array of obstacles that form a network of gaps, configured such that the first array and second array are oriented in different directions.

The device can include a second array of obstacles that form a network of gaps, wherein the first array is situated upstream of the second array, and wherein the second array has a higher density than the first array. In this configuration, the device can further include one or more additional arrays of obstacles that form a network of gaps, wherein each additional array has an equal or higher density than any array upstream of the additional array.

The binding moieties of the device can be ones that specifically bind a cell surface cancer marker, e.g., a cell surface cancer marker selected from Table 1 such as EpCAM, E-Cadherin, Mucin-1, Cytokeratin 8, EGFR, or leukocyte associated receptor (LAR).

The binding moieties can be a polypeptide such as an antibody, which, e.g., specifically binds one or more epithelial cells, cancer cells, bone marrow cells, fetal cells, progenitor cells, stem cells, foam cells, mesenchymal cells, immune system cells, endothelial cells, endometrial cells, connective tissue cells, trophoblasts, bacteria, fungi, or pathogens.

The device can include an inlet for delivery of the cellular sample and can be configured for delivery of a second fluid medium through the inlet; the fluid medium can include a buffer, a lysis reagent, a nucleic acid amplification reagent, an osmolarity regulating reagent, a labeling reagent, a preservative, or a fixing reagent.

The device can further include a programmable heating unit.

The device can be used to detect cancer cells in a cellular sample; the cellular sample is introduced into the device such that at least some of the obstacles that include one or more binding moieties come in contact with cells in the sample so that the presence or absence of cancer cells in the sample can be determined, and a diagnosis of cancer or lack of cancer can be made for the patient from whom the sample was obtained.

Any of the devices of the invention may be used together with a set of instructions for the device.

By “approximately equal” in the context of length, size, area, or other measurements is meant equal to within 10%, 5%, 4%, 3%, 2%, or even 1%.

By “biological particle” is meant any species of biological origin that is insoluble in aqueous media. Examples include cells, particulate cell components, viruses, and complexes including proteins, lipids, nucleic acids, and carbohydrates.

By “biological sample” is meant any sample of biological origin or containing, or potentially containing, biological particles. Preferred biological samples are cellular samples.

By “blood component” is meant any component of whole blood, including host red blood cells, white blood cells, platelets, or epithelial cells, in particular, CTCs. Blood components also include the components of plasma, e.g., proteins, lipids, nucleic acids, and carbohydrates, and any other cells that may be present in blood, e.g., because of current or past pregnancy, organ transplant, infection, injury, or disease.

By “cellular sample” is meant a sample containing cells or components thereof. Such samples include naturally occurring fluids (e.g., blood, sweat, tears, ear flow, sputum, lymph, bone marrow suspension, urine, saliva, semen, vaginal flow, cerebrospinal fluid, cervical lavage, brain fluid, ascites, milk, secretions of the respiratory, intestinal or genitourinary tract, amniotic fluid, and water samples) and fluids into which cells have been introduced (e.g., culture media and liquefied tissue samples). The term also includes a lysate.

By “channel” is meant a gap through which fluid may flow. A channel may be a capillary, a conduit, or a strip of hydrophilic pattern on an otherwise hydrophobic surface wherein aqueous fluids are confined.

By “circulating tumor cell” (CTC) is meant a cancer cell that is exfoliated from a solid tumor of a subject and is found in the subject's circulating blood.

By “component” of cell is meant any component of a cell that may be at least partially isolated upon lysis of the cell. Cellular components may be organelles (e.g., nuclei, perinuclear compartments, nuclear membranes, mitochondria, chloroplasts, or cell membranes), polymers or molecular complexes (e.g., lipids, polysaccharides, proteins (membrane, trans-membrane, or cytosolic), nucleic acids (native, therapeutic, or pathogenic), viral particles, or ribosomes), or other molecules (e.g., hormones, ions, cofactors, or drugs).

By “component” of a cellular sample is meant a subset of cells, or components thereof, contained within the sample.

By “density” in reference to an array of obstacles is meant the number of obstacles per unit of area, or alternatively the percentage of volume occupied by such obstacles. Array density is increased either by placing obstacles closer together or by increasing the size of obstacles relative to the gaps between obstacles.

By “enriched sample” is meant a sample containing components that has been processed to increase the relative population of components of interest relative to other components typically present in a sample. For example, samples may be enriched by increasing the relative population of cells of interest by at least 10%, 25%, 50%, 75%, 100% or by a factor of at least 1,000, 10,000, 100,000, 1,000,000, 10,000,000, or even 100,000,000.

By “exchange buffer” in the context of a cellular sample is meant a medium distinct from the medium in which the cellular sample is originally suspended, and into which one or more components of the cellular sample are to be exchanged.

By “flow-extracting boundary” is meant a boundary designed to remove fluid from an array.

By “flow-feeding boundary” is meant a boundary designed to add fluid to an array.

By “gap” is meant an opening through which fluids or particles may flow. For example, a gap may be a capillary, a space between two obstacles wherein fluids may flow, or a hydrophilic pattern on an otherwise hydrophobic surface wherein aqueous fluids are confined. In a preferred embodiment of the invention, the network of gaps is defined by an array of obstacles. In this embodiment, the gaps are the spaces between adjacent obstacles. In a preferred embodiment, the network of gaps is constructed with an array of obstacles on the surface of a substrate.

By “hydrodynamic size” is meant the effective size of a particle when interacting with a flow, obstacles, or other particles. It is used as a general term for particle volume, shape, and deformability in the flow.

By “hyperspectral” in reference to an imaging process or method is meant the acquisition of an image at five or more wavelengths or bands of wavelengths.

By “intracellular activation” is meant activation of second messenger pathways leading to transcription factor activation, or activation of kinases or other metabolic pathways. Intracellular activation through modulation of external cell membrane antigens may also lead to changes in receptor trafficking.

By “labeling reagent” is meant a reagent that is capable of binding to an analyte, being internalized or otherwise absorbed, and being detected, e.g., through shape, morphology, color, fluorescence, luminescence, phosphorescence, absorbance, magnetic properties, or radioactive emission.

By “microfluidic” is meant having at least one dimension of less than 1 mm.

By “microstructure” in reference to a surface is meant the microscopic structure of a surface that includes one or more individual features measuring less than 1 mm in at least one dimension. Exemplary microfeatures are micro-obstacles, micro-posts, micro-grooves, micro-fins, and micro-corrugation.

By “obstacle” is meant an impediment to flow in a channel, e.g., a protrusion from one surface. For example, an obstacle may refer to a post outstanding on a base substrate or a hydrophobic barrier for aqueous fluids. In some embodiments, the obstacle may be partially permeable. For example, an obstacle may be a post made of porous material, wherein the pores allow penetration of an aqueous component but are too small for the particles being separated to enter.

By “shrinking reagent” is meant a reagent that decreases the hydrodynamic size of a particle. Shrinking reagents may act by decreasing the volume, increasing the deformability, or changing the shape of a particle.

By “swelling reagent” is meant a reagent that increases the hydrodynamic size of a particle. Swelling reagents may act by increasing the volume, reducing the deformability, or changing the shape of a particle.

Other features and advantages will be apparent from the following description and the claims.

### BRIEF DESCRIPTION OF THE DRAWINGS

FIGS. 1A-1E are schematic depictions of an array that separates cells based on lateral displacement: (A) illustrates the lateral displacement of subsequent rows; (B) illustrates how fluid flowing through a gap is divided unequally around obstacles in subsequent rows; (C) illustrates how a particle with a hydrodynamic size above the critical size is displaced laterally in the device; (D) illustrates an array of cylindrical obstacles; and (E) illustrates an array of elliptical obstacles.

FIG. 2 is a schematic description illustrating the unequal division of the flux through a gap around obstacles in subsequent rows.

FIG. 3 is a schematic depiction of how the critical size depends on the flow profile, which is parabolic in this example.

FIG. 4 is an illustration of how shape affects the movement of particles through a device.

FIG. 5 is an illustration of how deformability affects the movement of particles through a device.

FIG. 6 is a schematic depiction of lateral displacement. Particles having a hydrodynamic size above the critical size move to the edge of the array, while particles having a hydrodynamic size below the critical size pass through the device without lateral displacement.

FIG. 7 is a schematic depiction of a three stage device.

FIG. 8 is a schematic depiction of the maximum size and cut-off size for the device of FIG. 7.

FIG. 9 is a schematic depiction of a bypass channel.

FIG. 10 is a schematic depiction of a bypass channel.

FIG. 11 is a schematic depiction of a three stage device having a common bypass channel.

FIG. 12 is a schematic depiction of a three stage, duplex device having a common bypass channel.

FIG. 13 is a schematic depiction of a three stage device having a common bypass channel, where the flow through the device is substantially constant.

FIG. 14 is a schematic depiction of a three stage, duplex device having a common bypass channel, where the flow through the device is substantially constant.

FIG. 15 is a schematic depiction of a three stage device having a common bypass channel, where the fluidic resistance in the bypass channel and the adjacent stage are substantially constant.

FIG. 16 is a schematic depiction of a three stage, duplex device having a common bypass channel, where the fluidic resistance in the bypass channel and the adjacent stage are substantially constant.

FIG. 17 is a schematic depiction of a three stage device having two, separate bypass channels.

FIG. 18 is a schematic depiction of a three stage device having two, separate bypass channels, which are in arbitrary configuration.

FIG. 19 is a schematic depiction of a three stage, duplex device having three, separate bypass channels.

FIG. 20 is a schematic depiction of a three stage device having two, separate bypass channels, wherein the flow through each stage is substantially constant.

FIG. 21 is a schematic depiction of a three stage, duplex device having three, separate bypass channels, wherein the flow through each stage is substantially constant.

FIG. 22 is a schematic depiction of a flow-extracting boundary.

FIG. 23 is a schematic depiction of a flow-feeding boundary.

FIG. 24 is a schematic depiction of a flow-feeding boundary, including a bypass channel.

FIG. 25 is a schematic depiction of two flow-feeding boundaries flanking a central bypass channel.

FIG. 26 is a schematic depiction of a device having four channels that act as on-chip flow resistors.

FIGS. 27 and 28 are schematic depictions of the effect of on-chip resistors on the relative width of two fluids flowing in a device.

FIG. 29 is a schematic depiction of a duplex device having a common inlet for the two outer regions.

FIG. 30A is a schematic depiction of a multiple arrays on a device. FIG. 30B is a schematic depiction of multiple arrays with common inlets and product outlets on a device.

FIG. 31 is a schematic depiction of a multi-stage device with a small footprint.

FIG. 32 is a schematic depiction of blood passing through a device.

FIG. 33A is a graph of cell count versus hydrodynamic size for a microfluidic separation of normal whole blood. FIG. 33B is a graph of cell count versus hydrodynamic size for a microfluidic separation of whole blood including a population of circulating tumor cells (CTCs). FIG. 33C is the graph of FIG. 33B, additionally showing a size cutoff that excludes most native blood cells. FIG. 33D is the graph of FIG. 33C, additionally showing a population of cells larger than the size cutoff and indicative of a disease state.

FIGS. 34A-34D are schematic depictions of moving a particle from a sample to a buffer in a single stage (A), three stage (B), duplex (C), or three stage duplex (D) device.

FIG. 35A is a schematic depiction of a two stage device employed to move a particle from blood to a buffer to produce three products. FIG. 35B is a schematic graph of the maximum size and cut off size of the two stages. FIG. 35C is a schematic graph of the composition of the three products.

FIG. 36 is a schematic depiction of a two stage device for alteration, where each stage has a bypass channel.

FIG. 37 is a schematic depiction of the use of fluidic channels to connect two stages in a device.

FIG. 38 is a schematic depiction of the use of fluidic channels to connect two stages in a device, wherein the two stages are configured as a small footprint array.

FIG. 39A is a schematic depiction of a two stage device having a bypass channel that accepts output from both stages. FIG. 39B is a schematic graph of the size range of product achievable with this device.

FIG. 40 is a schematic depiction of a two stage device for alteration having bypass channels that flank each stage and empty into the same outlet.

FIG. 41 is a schematic depiction of a device for the sequential movement and alteration of particles.

FIG. 42A is a schematic depiction of a device of the invention and its operation. FIG. 42B is an illustration of the device of FIG. 42A and a further-schematized representation of this device.

FIGS. 43A and 43B are schematic depictions of two distinct configurations for joining two devices together. In FIG. 43A, a cascade configuration is shown, in which outlet 1 of one device is joined to a sample inlet of a second device. In FIG. 43B, a bandpass configuration is shown, in which outlet 2 of one device is joined to a sample inlet of a second device.

FIG. 44 is a schematic depiction of an enhanced method of size separation in which target cells are labeled with immunoaffinity beads.

FIG. 45 is a schematic depiction of a method for performing size fractionation and for separating free labeling reagents, e.g., antibodies, from bound labeling reagents by using a device of the invention.

FIG. 46 is a schematic depiction of a method shown in FIG. 45. In this case, non-target cells may copurify with target cells, but these non-target cells do not interfere with quantification of target cells.

FIG. 47 is a schematic depiction of a method for enriching large cells from a mixture and producing a concentrated sample of these cells.

FIG. 48 is a schematic depiction of a method for lysing cells inside a device of the invention and separating whole cells from organelles and other cellular components.

FIG. 49 is a schematic depiction of two devices arrayed in a cascade configuration and used for performing size fractionation and for separating free labeling reagent from bound labeling reagent by using a device of the invention.

FIG. 50 is a schematic depiction of two devices arrayed in a cascade configuration and used for performing size fractionation and for separating free labeling reagent from bound labeling reagent by using a device of the invention. In this figure, phage is utilized for binding and detection rather than antibodies.

FIG. 51 is a schematic depiction of two devices arrayed in a bandpass configuration.

FIG. 52 is a graph of cell count versus hydrodynamic size for a microfluidic separation of normal whole blood.

FIG. 53 is a set of histograms from input, product, and waste samples generated with a Coulter “A^{C}-T diff” clinical blood analyzer. The x-axis depicts cell volume in femtomoles.

FIG. 54 is a pair of representative micrographs from product and waste streams of fetal blood processed with a cell enrichment module, showing clear separation of nucleated cells and red blood cells.

FIG. 55 is a pair of images showing cells fixed on a cell enrichment module with paraformaldehyde and observed by fluorescence microscopy. Target cells are bound to the obstacles and floor of the capture module.

FIG. 56 is a schematic depiction of a method of the invention. This method features isolating and counting large cells within a cellular sample, wherein the count is indicative of a patient's disease state, and subsequently further analyzing the large cell subpopulation.

FIG. 57A is a design for a preferred embodiment of the invention. FIG. 57B is a table of design parameters corresponding to FIG. 57A. FIG. 57C is a mask design of a chip of the invention.

FIG. 58 is a schematic depiction of a method of detecting epidermal growth factor receptor (EGFR) mutations in CTCs in blood.

FIG. 59 is a schematic depiction of a process for generating EGFR sequencing templates. EGFR mRNA is reverse transcribed to make cDNA; next, two PCR amplifications are performed sequentially.

FIG. 60 is a schematic depiction of an allele-specific TaqMan 5′ Nuclease Real Time PCR assay used to amplify EGFR subregions specific to particular mutations of interest.

FIG. 61 is a set of sequencing charts showing the detection of several EGFR mutations. (shaded regions) above the background level of fluorescence.

FIG. 62A is an image of an agarose gel showing that EpCAM and EGFR are expressed in tumor cells but not in leukocytes. BCKDK is expressed in both types of cells, while CD45 is expressed only in leukocytes. FIG. 62B is a graph and table showing a Pharmagene XpressWay™ profile of EGFR mRNA expression. Expression levels are profiled in 72 tissues via quantitative RT-PCR, and >10,000 copies per cell are detected in almost every tissue profiled except for blood. The table shows quantitation of mRNA for tissues #1-4 from the graph.

FIG. 63 is a pair of images of agarose gels showing the results of a two sets of PCR assays. In the first set (left), PCR is performed on EGFR input RNA at various concentrations. In the second set of assays, samples from the first set of PCR reactions are amplified with nested primers.

FIG. 64A is an image of an agarose gel showing the results of a set of PCR assays in which NCI-H1975 RNA is mixed with various quantities of peripheral blood mononuclear cell (PBMC) RNA and reverse transcribed prior to PCR. Spurious amplification bands are seen at the highest dilution. FIG. 64B is an image of an agarose gel showing the results of a set of PCR assays in which the samples shown in FIG. 64A are further amplified using nested primers. No spurious amplification bands are produced, even at the highest dilution.

FIG. 65 is an image of an agarose gel showing the results of a set of PCR assays. In the associated experiment, whole blood spiked with H1650 cells was run on two devices of the invention, and cDNA was synthesized from the resulting enriched samples. PCR using EGFR and CD45 primers was performed. Both wild type (138 bp) and mutant (123 bp) EGFR bands are visible in the lanes showing EGFR amplifications.

FIG. 66A is a schematic depiction of an array of the invention containing staggered subarrays. FIG. 66B is a schematic depiction contrasting a regular array with a staggered array. FIG. 66C is a schematic depiction showing the flow and capture of cells in a staggered array. FIG. 66D is a schematic depiction showing a device containing an outlet port surrounded by a region of narrowed flow paths. FIG. 66E is a schematic depiction of a device that is structured in the depth dimension to create narrowed flow paths. FIG. 66F is a schematic depiction of the device of FIG. 66E, showing captured cells. FIG. 66G is a set of microscope views showing stained H1650 cells captured in the narrow flow regions of a device of the invention.

FIG. 67A is a chart and inset showing the size distribution of several cellular samples, including white blood cells and various cancer cell lines, as measured by a Beckman Coulter Z2 counting device. The main chart uses a logarithmic scale for the volume axis, while the inset uses a linear scale to better represent the distribution of white blood cells. FIG. 67B is a chart showing the size distribution of several cancer cell lines. FIG. 67C is the chart of FIG. 67B, further showing three exemplary size cutoffs.

FIG. 68A is a schematic depiction of a capture device of the invention that features a functionalized microscope slide on the bottom of a sample chamber. FIG. 68B is a schematic depiction of a method of rocking cells in the capture device in order to keep the cells tumbling and prevent sedimentation. FIG. 68C is a schematic depiction of a method of rotating the capture device as an alternative to rocking. FIG. 68D is a schematic depiction of a capture device that includes two additional fluid chambers, which may be alternately filled and emptied in order to cause fluid motion inside the main chamber of the device. FIG. 68E is a schematic depiction of a microscope slide with multiple, spatially patterned capture functionalities on the surface.

FIG. 69A is a schematic depiction of a centrifugation device of the invention, shown both at rest and in operation. FIG. 69B is a schematic depiction of a cell binding to a functionalized surface in a gravitational field (top) and a centrifugal field (bottom). FIG. 69C is a schematic depiction of the device of FIG. 69A in which the chambers are inverted during the spin. FIG. 69D is a schematic depiction of the device of FIG. 69C, further showing a second functionalized surface for the capture of contaminating cells. FIG. 69E is a schematic depiction of a centrifugal device in which the functionalized slide is inclined at an angle during the spin. FIG. 69F is a pair of charts showing spin speed versus operating time, including periods that may be optimized: “spin up” (1), “spin time” (2), “spin down” (3), and rest time (4). FIG. 69G is a schematic depiction of a centrifugal device that includes a functionalized microstructure surface.

FIG. 70A is an image of an enrichment device showing the flow paths of a small cell (left) and a large cell (right). The small cell may be seen to have very little interaction with the obstacles and flows essentially in the average flow direction, while the large cell contacts each obstacle along its path and is directed laterally through the array. FIG. 70B is a schematic depiction of a device of the invention containing a regular array of obstacles. FIG. 70C is a schematic depiction of a device of the invention that includes multiple arrays in which the direction of deflection, the gap size, and/or the distance between obstacles is varied throughout the device, while the critical size is kept constant.

Figures are not necessarily to scale.

### DETAILED DESCRIPTION OF THE INVENTION

The invention features devices and methods for detecting, enriching, and analyzing circulating tumor cells (CTCs) and other particles. The invention further features methods of diagnosing a condition in a subject, e.g., cancer, by analyzing a cellular sample from the subject. In some embodiments, devices of the invention include arrays of obstacles that allow displacement of CTCs or other fluid components.

While this application focuses primarily on the detection, enrichment, and analysis of CTCs or epithelial cells, the devices and methods of the invention are useful for processing a wide range of other cells and particles, e.g., red blood cells, white blood cells, fetal cells, stem cells (e.g., undifferentiated), bone marrow cells, progenitor cells, foam cells, mesenchymal cells, endothelial cells, endometrial cells, trophoblasts, cancer cells, immune system cells (host or graft), connective tissue cells, bacteria, fungi, cellular pathogens (e.g., bacterial or protozoa), cellular organelles and other cellular components (e.g., mitochondria and nuclei), and viruses.

Exemplary devices and methods of the invention are described in detail below.

Circulating Tumor Cells (CTCs)

Epithelial cells that are exfoliated from solid tumors have been found in very low concentrations in the circulation of patients with advanced cancers of the breast, colon, liver, ovary, prostate, and lung, and the presence or relative number of these cells in blood has been correlated with overall prognosis and response to therapy. These CTCs may be an early indicator of tumor expansion or metastasis before the appearance of clinical symptoms.

CTCs typically have a short half-life of approximately one day, and their presence generally indicates a recent influx from a proliferating tumor. Therefore, CTCs represent a dynamic process that may reflect the current clinical status of patient disease and therapeutic response. Enumeration and characterization of CTCs, using the devices and methods of the invention, is useful in assessing cancer prognosis and in monitoring therapeutic efficacy for early detection of treatment failure that may lead to disease relapse. In addition, CTC analysis according to the invention enables the detection of early relapse in presymptomatic patients who have completed a course of therapy.

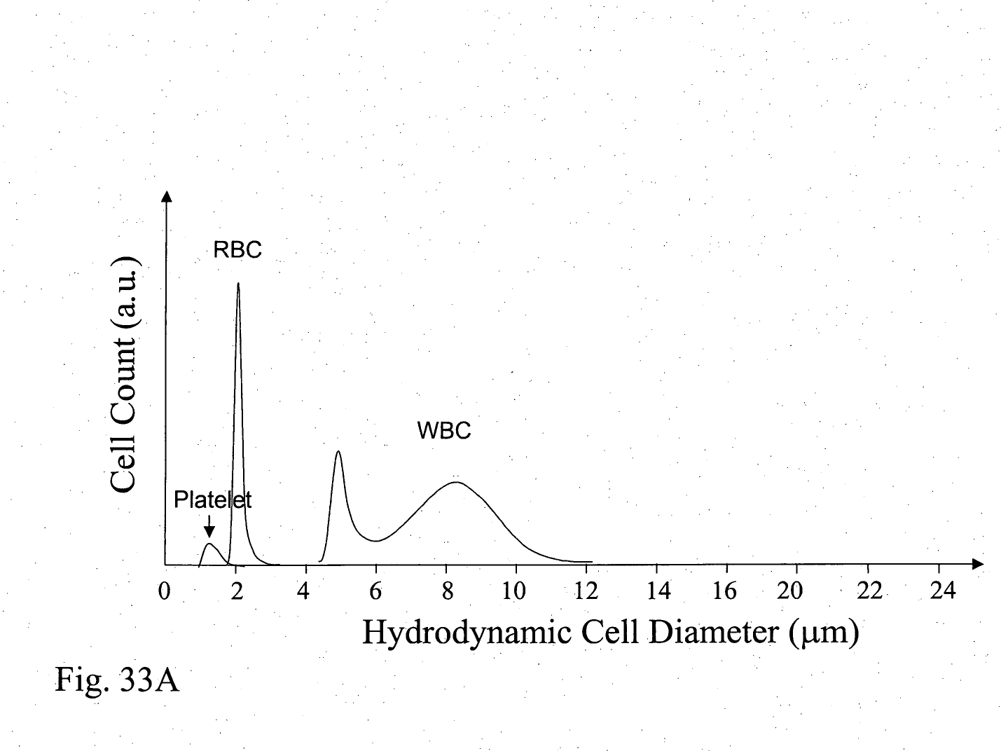

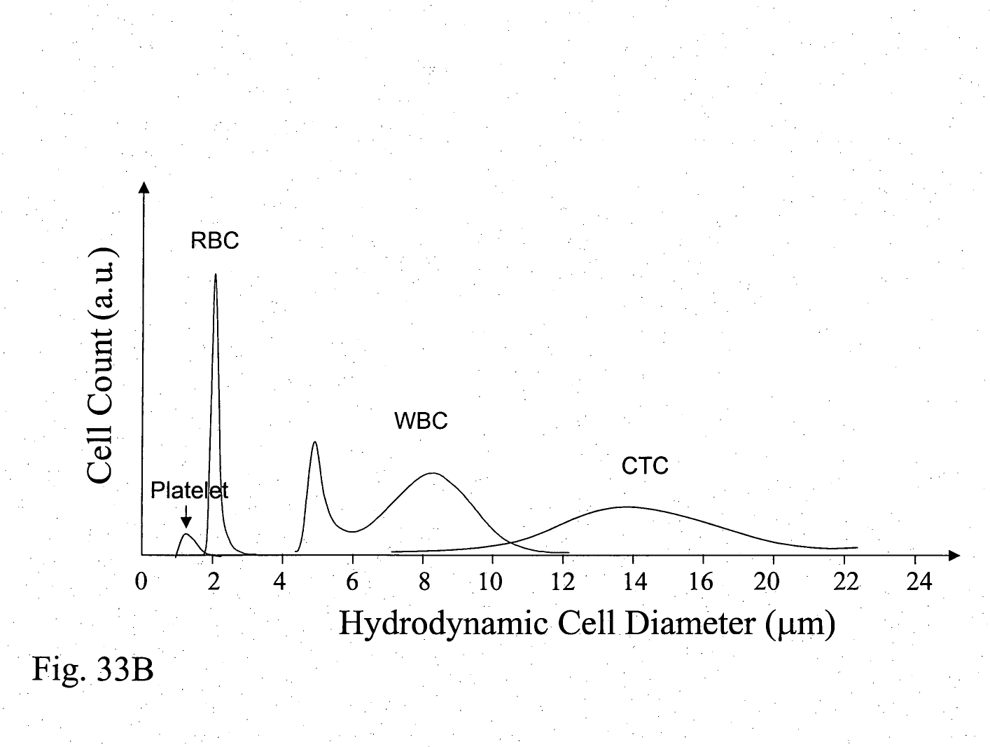

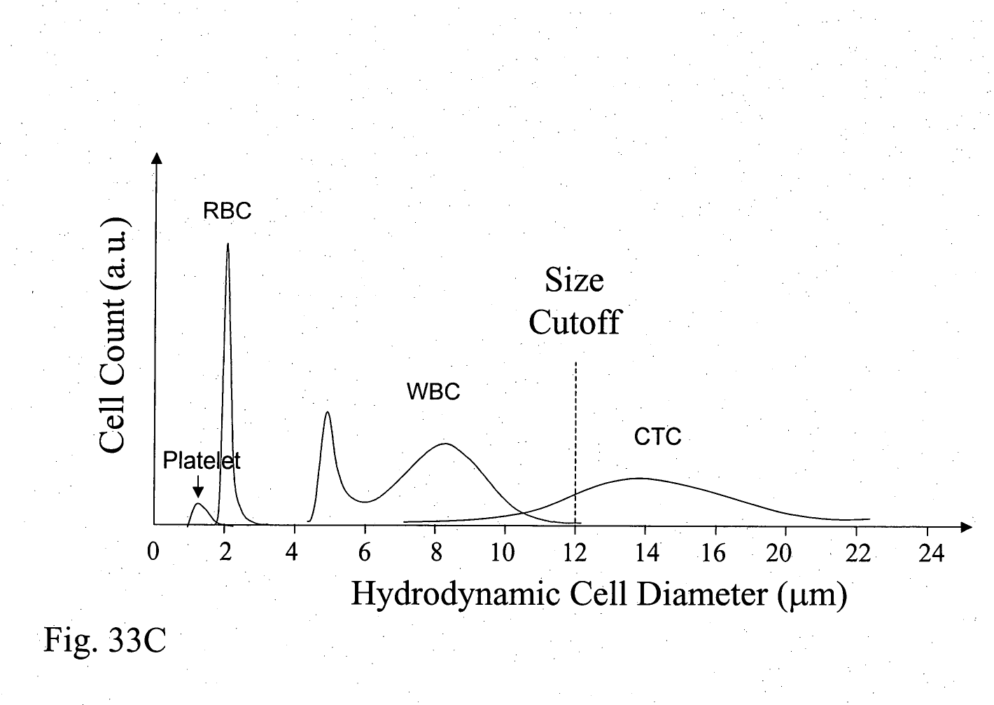

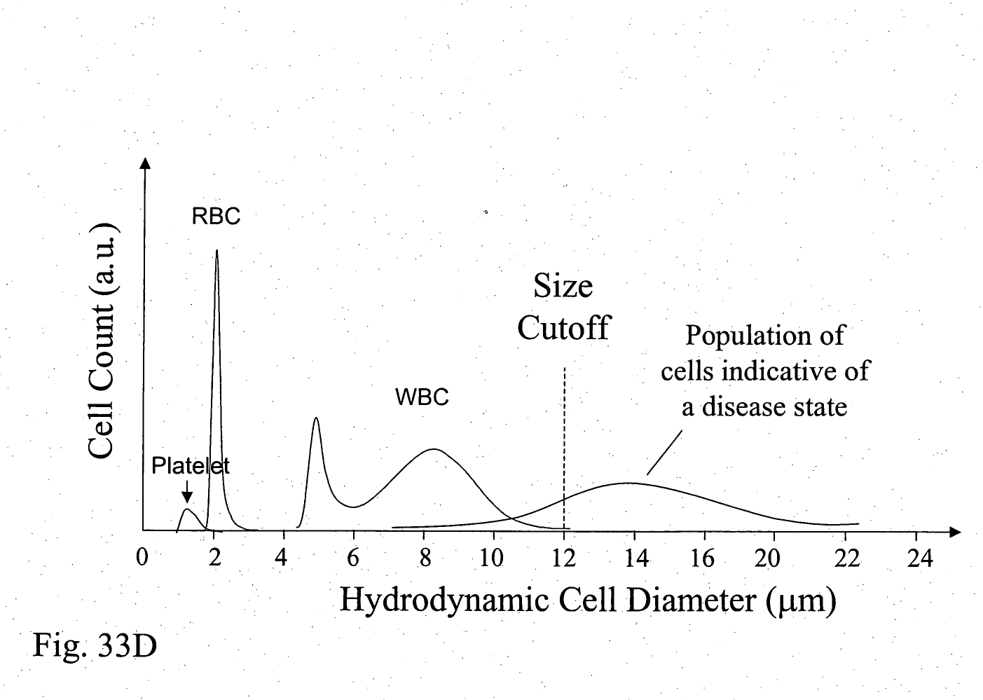

CTCs are generally larger than most blood cells (see, e.g., FIG. 33B). Therefore, one useful approach for analyzing CTCs in blood is to enrich cells based on size, resulting in a cell population enriched in CTCs. This cell population may then be subjected to further processing or analysis. Other methods of enrichment of CTCs are also possible using the invention. Devices and methods for enriching, enumerating, and analyzing CTCs are described below.

Device

In general, the devices include one or more arrays of obstacles that allow lateral displacement of CTCs and other components of fluids, thereby offering mechanisms of enriching or otherwise processing such components. Prior art devices that differ from those the present invention, but which, like those of the invention, employ obstacles for this purpose, are described, e.g., in Huang et al. Science 304, 987-990 (2004) and U.S. Publication No. 20040144651. The devices of the invention for separating particles according to size typically employ an array of a network of gaps, wherein a fluid passing through a gap is divided unequally into subsequent gaps. The array includes a network of gaps arranged such that fluid passing through a gap is divided unequally, even though the gaps may be identical in dimensions. The method uses a flow that carries cells to be separated through the array of gaps. The flow is aligned at a small angle (flow angle) with respect to a line-of-sight of the array. Cells having a hydrodynamic size larger than a critical size migrate along the line-of-sight, i.e., laterally, through the array, whereas those having a hydrodynamic size smaller than the critical size follow the average flow direction. Flow in the device occurs under laminar flow conditions. Devices of the invention are optionally configured as continuous-flow devices.

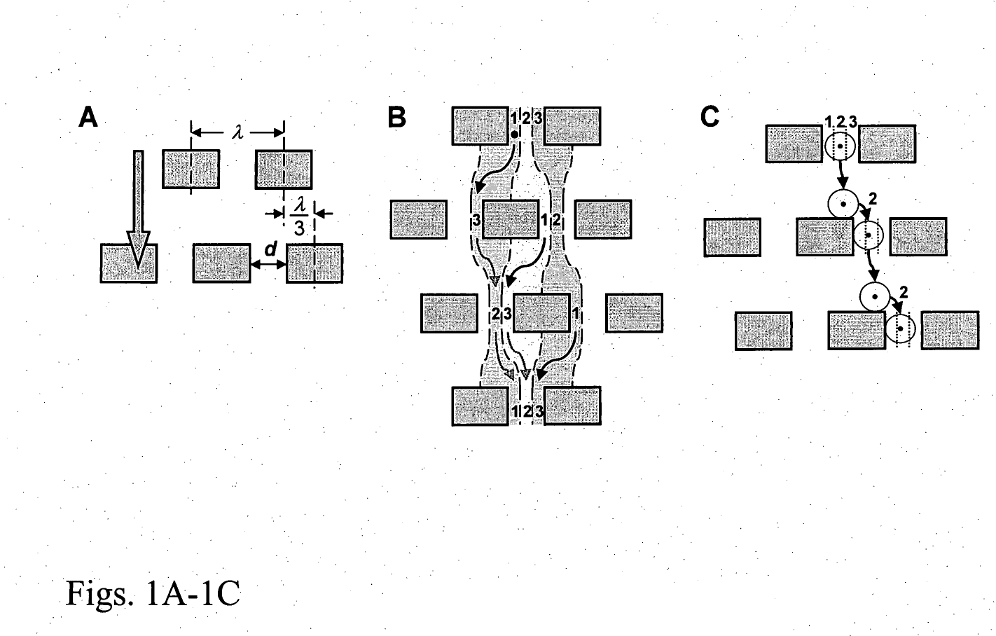

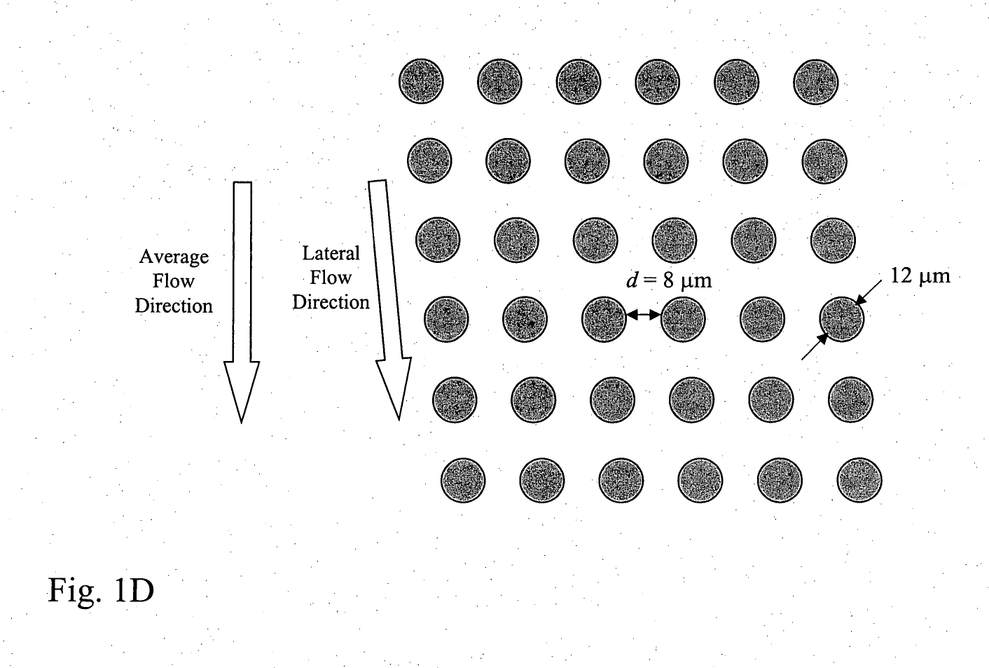

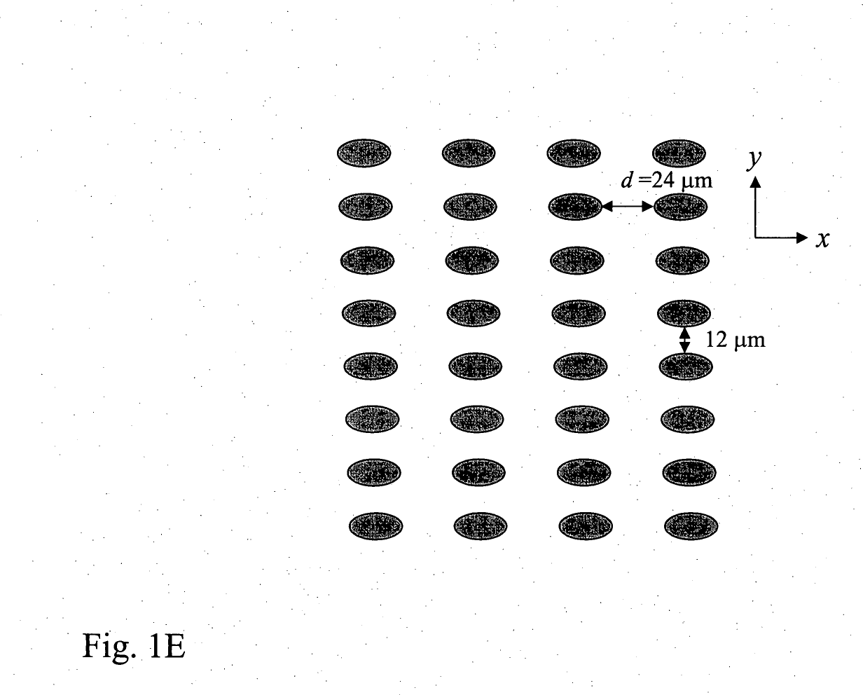

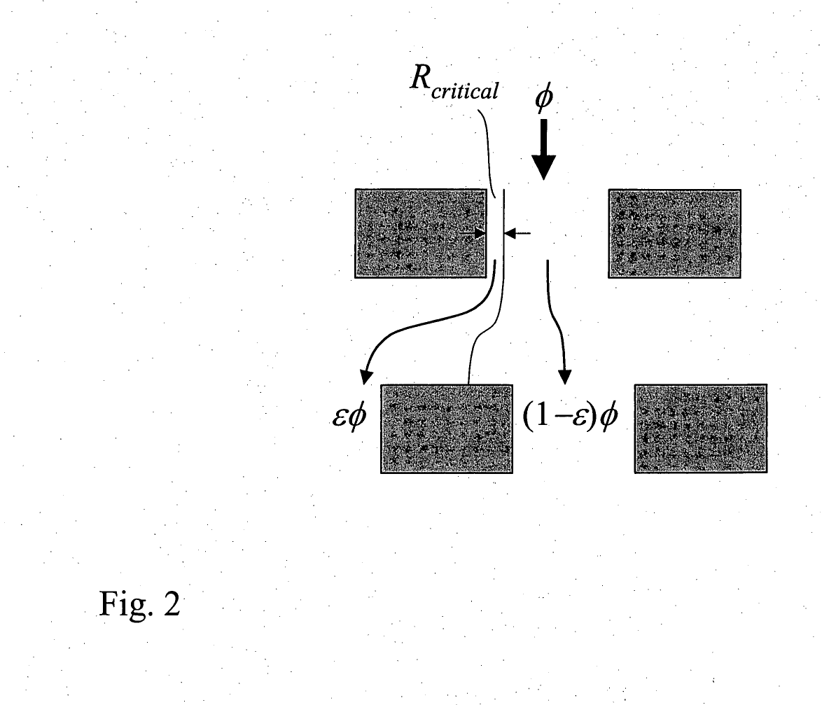

The critical size is a function of several design parameters. With reference to the obstacle array in FIGS. 1A-1C, each row of obstacles is shifted horizontally with respect to the previous row by Δλ, where λ is the center-to-center distance between the obstacles (FIG. 1A). The parameter Δλ/λ (the “bifurcation ratio,” ε) determines the ratio of flow bifurcated to the left of the next obstacle. In FIGS. 1A-1C, ε is 1/3, for the convenience of illustration. In general, if the flux through a gap between two obstacles is φ, the minor flux is εφ, and the major flux is (1−ε)φ (FIG. 2). In this example, the flux through a gap is divided essentially into thirds (FIG. 1B). While each of the three fluxes through a gap weaves around the array of obstacles, the average direction of each flux is in the overall direction of flow. FIG. 1C illustrates the movement of particles sized above the critical size through the array. Such particles move with the major flux, being transferred sequentially to the major flux passing through each gap.

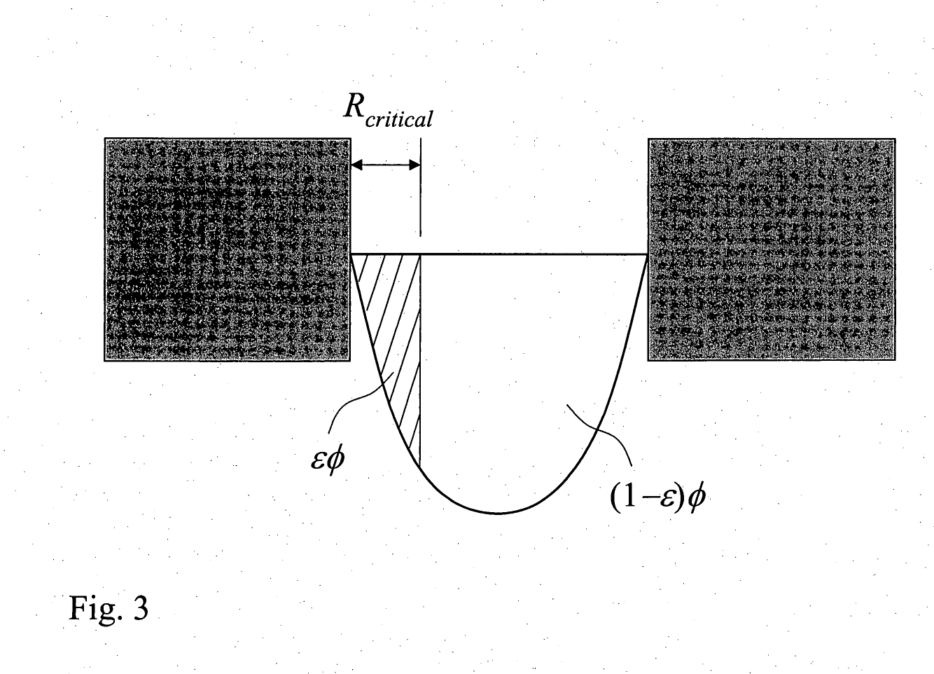

Referring to FIG. 2, the critical size is approximately 2R_{critical}, where R_{critical} is the distance between the stagnant flow line and the obstacle. If the center of mass of a particle, e.g., a cell, falls within R_{critical}, the particle would follow the major flux and move laterally through the array. R_{critical} may be determined if the flow profile across the gap is known (FIG. 3); it is the thickness of the layer of fluids that would make up the minor flux. For a given gap size, d, R_{critical} may be tailored based on the bifurcation ratio, ε. In general, the smaller ε, the smaller R_{critical}.

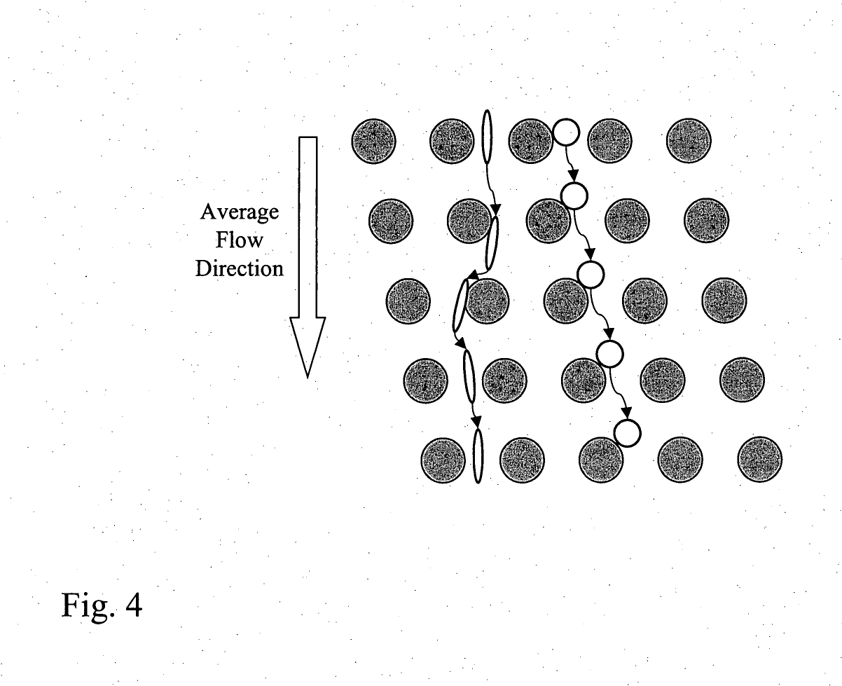

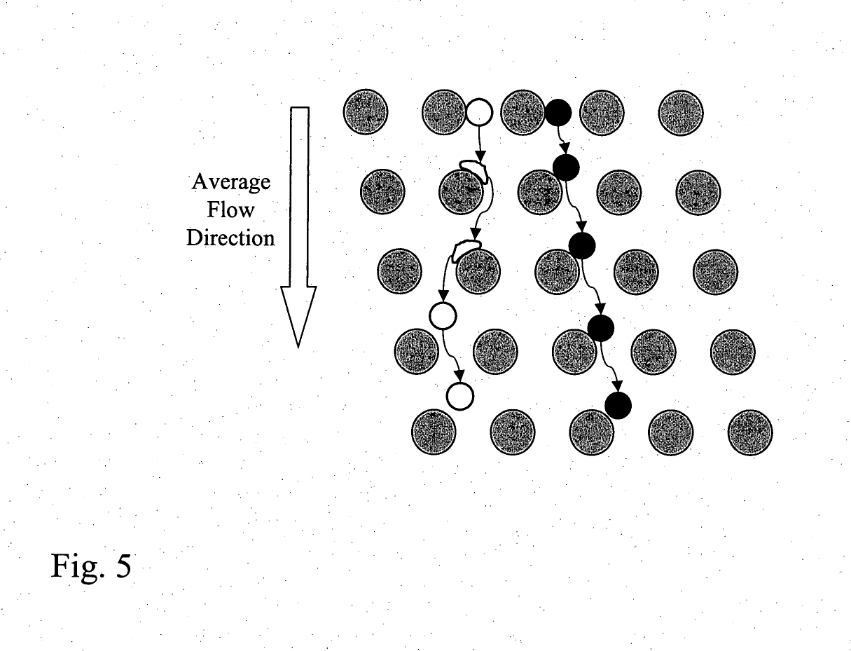

In an array for lateral displacement, particles of different shapes behave as if they have different sizes (FIG. 4). For example, lymphocytes are spheres of ˜5 μm diameter, and erythrocytes are biconcave disks of ˜7 μm diameter, and ˜1.5 μm thick. The long axis of erythrocytes (diameter) is larger than that of the lymphocytes, but the short axis (thickness) is smaller. If erythrocytes align their long axes to a flow when driven through an array of obstacles by the flow, their hydrodynamic size is effectively their thickness (˜1.5 μm), which is smaller than lymphocytes. When an erythrocyte is driven through an array of obstacles by a hydrodynamic flow, it tends to align its long axis to the flow and behave like a ˜1.5 μm-wide particle, which is effectively “smaller” than lymphocytes. The method and device may therefore separate cells according to their shapes, although the volumes of the cells could be the same. In addition, particles having different deformability behave as if they have different sizes (FIG. 5). For example, two particles having the same undeformed shape may be separated by lateral displacement, as the cell with the greater deformability may deform when it comes into contact with an obstacle in the array and change shape. Thus, separation in the device may be achieved based on any parameter that affects hydrodynamic size including the physical dimensions, the shape, and the deformability of the particle.

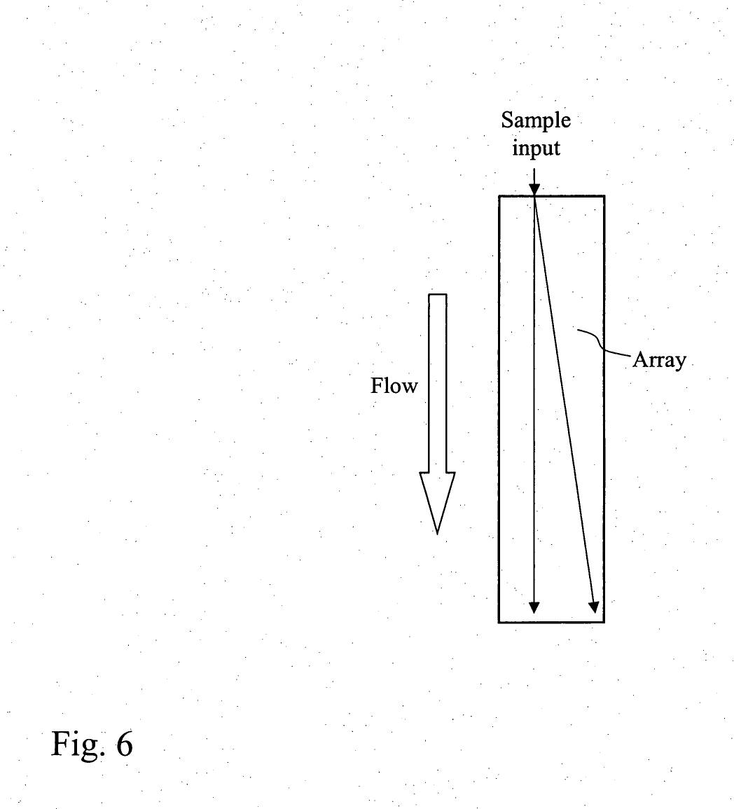

Referring to FIG. 6, feeding a mixture of particles, e.g., cells, of different hydrodynamic sizes from the top of the array and collecting the particles at the bottom, as shown schematically, produces two outputs, the product containing cells larger than the critical size, 2R_{critical}, and waste containing cells smaller than the critical size. Although labeled “waste” in FIG. 6, particles below the critical size may be collected while the particles above the critical size are discarded. Both types of outputs may also be desirably collected, e.g., when fractionating a sample into two or more sub-samples. Cells larger than the gap size will get trapped inside the array. Therefore, an array has a working size range. Cells have to be larger than a cut-off size (2R_{critical}) and smaller than a maximum pass-through size (array gap size) to be directed into the major flux. The “size range” of an array is defined as the ratio of maximum pass-through size to cut-off size.

In some cases, the gaps between obstacles are more than 15 microns, more than 20 microns, or less than 60 microns in size. In other cases, the gaps are between 20 and 100 microns in size.

In certain embodiments, a device of the invention may contain obstacles that include binding moieties, e.g., monoclonal anti-EpCAM antibodies or fragments thereof, that selectively bind to particular cell types, e.g., cells of epithelial origin, e.g., tumor cells. All of the obstacles of the device may include these binding moieties; alternatively, only a subset of the obstacles include them. Devices may also include additional modules that are fluidically coupled, e.g., a cell counting module or a detection module. For example, the detection module may be configured to visualize an output sample of the device. In addition, devices of the invention may be configured to direct cells in a selected size range in one direction, and other cells in a second direction. For example, the device may be configured to enrich cells having a hydrodynamic size greater than 12 microns, 14 microns, 16 microns, 18 microns, or even 20 microns from smaller cells in the sample. Alternatively, the device may enrich cells having a hydrodynamic size greater than or equal to 6 microns and less than or equal to 12 microns, e.g., cells having a hydrodynamic size greater than or equal to 8 microns and less than or equal to 10 microns, from other cells. The device may also enrich cells having a hydrodynamic size greater than or equal to 5 microns and less than or equal to 10 microns from cells having a hydrodynamic size greater than 10 microns; alternatively, it may enrich cells having a hydrodynamic size greater than or equal to 4 microns and less than or equal to 8 microns from cells having a hydrodynamic size greater than 8 microns. In general, the device may be configured to separate two groups of cells, where the first group has a larger average hydrodynamic size than the second group.

In some embodiments, devices of the invention may process more than 20 mL of fluid per hour, or even 50 mL of fluid per hour.

As described above, a device of the invention typically contains an array of obstacles that form a network of gaps. For example, such a device may include a staggered two-dimensional array of obstacles, e.g., such that each successive row is offset by less than half of the period of the previous row. The device may also include a second staggered two-dimensional array of obstacles, which is optionally oriented in a different direction than the first array. In this case, the first array may be situated upstream of the second array, and the second array may have a higher density than the first array. Multiple arrays may be configured in this manner, such that each additional array has an equal or higher density than any array upstream of the additional array.

Devices of the invention may be adapted for implantation in a subject. For example, such a device may be adapted for placement in or near the circulatory system of a subject in order to be able to process blood samples. Such devices may be part of an implantable system of the invention that is fluidically coupled to the circulatory system of a subject, e.g., through tubing or an arteriovenous shunt. In some cases, systems of the invention that include implantable devices, e.g., disposable systems, may remove one or more analytes, components, or materials from the circulatory system. These systems may be adapted for continuous blood flow through the device.

Sample Mobilization Devices

The invention additionally encompasses devices for cell enrichment, e.g., enrichment of CTCs, that employ sample mobilization. A sample mobilization device gives rise to movement of cells, or other components of a fluid sample, relative to features, e.g., obstacles, of the device. For example, one device of the invention includes a receptacle that may hold a cellular sample, a detachably attached lid configured to fit within the receptacle that includes a functionalized lid surface including one or more capture moieties that selectively capture cells of interest, and an sample mobilizer coupled to either the receptacle or the lid. Optionally, the receptacle has a functionalized surface including one or more capture moieties that selectively capture a second cell type. The lid surface may have any shape, e.g., square, rectangular, or circular. The device may be manufactured using any materials known in the art, e.g., glass, silicon, or plastic. In some cases, the lid surface or receptacle surface includes a microstructure, e.g., a micro-obstacle, a micro-corrugation, a micro-groove, or a micro-fin. The capture moieties may include one or more antibodies that specifically bind to a particular cell type, and these antibodies may be configured in an array. As with other devices of the invention, the antibodies may specifically bind to any of a wide variety of cells, e.g., leukocytes or epithelial cells. Preferably, the antibodies are able to bind specifically to CTCs. Furthermore, the antibodies may specifically bind a cell surface cancer marker, e.g., EpCAM, E-Cadherin, Mucin-1, Cytokeratin 8, epidermal growth factor receptor (EGFR), and leukocyte associated receptor (LAR), or a marker selected from Table 1. In some cases, the lid of a sample mobilization device may be designed to fit into the receptacle at a nonorthogonal angle with respect to a wall of the receptacle. The receptacle may be designed to hold any desirable amount of sample, e.g., 10 mL or 50 mL.

TABLE 1

| | | |
|---|---|---|
| 2AR | BETA 1 INTEGRIN SUBUNIT | CD10 |
| A DISINTEGRIN | BETA 3 INTEGRIN SUBUNIT | CD146 |
| ACTIVATOR OF THYROID | BETA 5 INTEGRIN SUBUNIT | CD147 |
| AND RETINOIC ACID | BETA-2 INTERFERON | CD24 |
| RECEPTOR (ACTR) | BETA-CATENIN | CD29 |
| ADAM 11 | BETA-CATENIN | CD44 |
| ADIPOGENESIS | BONE SIALOPROTEIN | CD51 |
| INHIBITORY FACTOR | (BSP) | CD54 |
| (ADIF) | BREAST CANCER | CD61 |
| ALPHA 6 INTEGRIN | ESTROGEN-INDUCIBLE | CD66e |
| SUBUNIT | SEQUENCE (BCEI) | CD82 |
| ALPHA V INTEGRIN | BREAST CANCER | CD87 |
| SUBUNIT | RESISTANCE PROTEIN | CD9 |
| ALPHA-CATENIN | (BCRP) | CEA |
| AMPLIFIED IN BREAST | BREAST CANCER TYPE 1 | CELLULAR RETINOL- |
| CANCER 1 (AIB1) | (BRCA1) | BINDING PROTEIN 1 |
| AMPLIFIED IN BREAST | BREAST CANCER TYPE 2 | (CRBP1) |
| CANCER 3 (AIB3) | (BRCA2) | c-ERBB-2 |
| AMPLIFIED IN BREAST | BREAST CARCINOMA | CK7 |
| CANCER 4 (AIB4) | AMPLIFIED SEQUENCE 2 | CK8 |
| AMYLOID PRECURSOR | (BCAS2) | CK18 |
| PROTEIN SECRETASE | CADHERIN | CK19 |
| (APPS) | EPITHELIAL CADHERIN-11 | CK20 |
| AP-2 GAMMA | CADHERIN-ASSOCIATED | CLAUDIN-7 |
| APPS | PROTEIN | c-MET |
| ATP-BINDING CASSETTE | CALCITONIN RECEPTOR | COLLAGENASE |
| TRANSPORTER (ABCT) | (CTR) | FIBROBLAST |
| PLACENTA-SPECIFIC | CALCIUM PLACENTAL | COLLAGENASE |
| (ABCP) | PROTEIN (CAPL) | INTERSTITIAL |
| ATP-BINDING CASSETTE | CALCYCLIN | COLLAGENASE-3 |
| SUBFAMILY C MEMBER | CALLA | COMMON ACUTE |
| (ABCC1) | CAM5 | LYMPHOCYTIC LEUKEMIA |
| BAG-1 | CAPL | ANTIGEN (CALLA) |
| BASIGIN (BSG) | CARCINOEMBRYONIC | CONNEXIN 26 (Cx26) |
| BCEI | ANTIGEN (CEA) | CONNEXIN 43 (Cx43) |
| B-CELL DIFFERENTIATION | CATENIN | CORTACTIN |
| FACTOR (BCDF) | ALPHA 1 | COX-2 |
| B-CELL LEUKEMIA 2 (BCL- | CATHEPSIN B | CTLA-8 |
| 2) | CATHEPSIN D | CTR |
| B-CELL STIMULATORY | CATHEPSIN K | CTSD |
| FACTOR-2 (BSF-2) | CATHEPSIN L2 | CYCLIN D1 |
| BCL-1 | CATHEPSIN O | CYCLOOXYGENASE-2 |
| BCL-2-ASSOCIATED X | CATHEPSIN O1 | HEPATOCYTE- |
| PROTEIN (BAX) | CATHEPSIN V | STIMULATING FACTOR III |
| BCRP | FIBRONECTIN RECEPTOR | (HSF III) |
| CYTOKERATIN 18 | BETA SUBUNIT (FNRB) | HER-2 |
| CYTOKERATIN 19 | FLK-1 | HER2/NEU |
| CYTOKERATIN 8 | GA15.3 | HERMES ANTIGEN |
| CYTOTOXIC T- | GA733.2 | HET |
| LYMPHOCYTE- | GALECTIN-3 | HHM |
| ASSOCIATED SERINE | GAMMA-CATENIN | HUMORAL |
| ESTERASE 8 (CTLA-8) | GAP JUNCTION PROTEIN | HYPERCALCEMIA OF |
| DIFFERENTIATION- | (26 kDa) | MALIGNANCY (HHM) |
| INHIBITING ACTIVITY (DIA) | GAP JUNCTION PROTEIN | ICERE-1 |
| DNA AMPLIFIED IN | (43 kDa) | INT-1 |
| MAMMARY CARCINOMA 1 | GAP JUNCTION PROTEIN | INTERCELLULAR |
| (DAM1) | ALPHA-1 (GJA1) | ADHESION MOLECULE-1 |
| DNA TOPOISOMERASE II | GAP JUNCTION PROTEIN | (ICAM-1) |
| ALPHA | BETA-2 (GJB2) | INTERFERON-GAMMA- |
| DR-NM23 | GCP1 | INDUCING FACTOR (IGIF) |
| E-CADHERIN | GELATINASE A | INTERLEUKIN-1 ALPHA (IL- |
| EMMPRIN | GELATINASE B | 1A) |
| EMS1 | GELATINASE (72 kDa) | INTERLEUKIN-1 BETA (IL- |
| ENDOTHELIAL CELL | GELATINASE (92 kDa) | 1B) |
| GROWTH FACTOR (ECGR) | GLIOSTATIN | INTERLEUKIN-11 (IL-11) |
| PLATELET-DERIVED (PD- | GLUCOCORTICOID | INTERLEUKIN-17 (IL-17) |
| ECGF) | RECEPTOR INTERACTING | INTERLEUKIN-18 (IL-18) |
| ENKEPHALINASE | PROTEIN 1 (GRIP1) | INTERLEUKIN-6 (IL-6) |
| EPIDERMAL GROWTH | GLUTATHIONE S- | INTERLEUKIN-8 (IL-8) |
| FACTOR RECEPTOR | TRANSFERASE p | INVERSELY CORRELATED |
| (EGFR) | GM-CSF | WITH ESTROGEN |
| EPISIALIN | GRANULOCYTE | RECEPTOR EXPRESSION- |
| EPITHELIAL MEMBRANE | CHEMOTACTIC PROTEIN 1 | 1 (ICERE-1) |
| ANTIGEN (EMA) | (GCP1) | KAI1 |
| ER-ALPHA | GRANULOCYTE- | KDR |
| ERBB2 | MACROPHAGE-COLONY | KERATIN 8 |
| ERBB4 | STIMULATING FACTOR | KERATIN 18 |
| ER-BETA | GROWTH FACTOR | KERATIN 19 |
| ERF-1 | RECEPTOR BOUND-7 | KISS-1 |
| ERYTHROID- | (GRB-7) | LEUKEMIA INHIBITORY |
| POTENTIATING ACTIVITY | GSTp | FACTOR (LIF) |
| (EPA) | HAP | LIF |
| ESR1 | HEAT-SHOCK COGNATE | LOST IN INFLAMMATORY |
| ESTROGEN RECEPTOR- | PROTEIN 70 (HSC70) | BREAST CANCER (LIBC) |
| ALPHA | HEAT-STABLE ANTIGEN | LOT (“LOST ON |
| ESTROGEN RECEPTOR- | HEPATOCYTE GROWTH | TRANSFORMATION”) |
| BETA | FACTOR (HGF) | LYMPHOCYTE HOMING |
| ETS-1 | HEPATOCYTE GROWTH | RECEPTOR |
| EXTRACELLULAR MATRIX | FACTOR RECEPTOR | MACROPHAGE-COLONY |
| METALLOPROTEINASE | (HGFR) | STIMULATING FACTOR |
| INDUCER (EMMPRIN) | MULTIDRUG RESISTANCE | MAGE-3 |
| FIBRONECTIN RECEPTOR | PROTEIN 1 (MDR, MDR1) | PAI-2 |
| BETA POLYPEPTIDE | MULTIDRUG RESISTANCE | PARATHYROID |
| (FNRB) | RELATED PROTEIN-1 | ADENOMATOSIS 1 (PRAD1) |
| MAMMAGLOBIN | (MRP, MRP-1) | PARATHYROID HORMONE- |
| MASPIN | N-CADHERIN | LIKE HORMONE (PTHLH) |
| MC56 | NEP | PARATHYROID HORMONE- |
| M-CSF | NEU | RELATED PEPTIDE (PTHrP) |
| MDC | NEUTRAL | P-CADHERIN |
| MDNCF | ENDOPEPTIDASE | PD-ECGF |
| MDR | NEUTROPHIL-ACTIVATING | PDGF-β |
| MELANOMA CELL | PEPTIDE 1 (NAP1) | PEANUT-REACTIVE |
| ADHESION MOLECULE | NM23-H1 | URINARY MUCIN (PUM) |
| (MCAM) | NM23-H2 | P-GLYCOPROTEIN (P-GP) |
| MEMBRANE | NME1 | PGP-1 |
| METALLOENDOPEPTIDASE | NME2 | PHGS-2 |
| (MME) | NUCLEAR RECEPTOR | PHS-2 |
| MEMBRANE-ASSOCIATED | COACTIVATOR-1 (NCoA-1) | PIP |
| NEUTRAL | NUCLEAR RECEPTOR | PLAKOGLOBIN |
| ENDOPEPTIDASE (NEP) | COACTIVATOR-2 (NCoA-2) | PLASMINOGEN |
| CYSTEINE-RICH PROTEIN | NUCLEAR RECEPTOR | ACTIVATOR INHIBITOR |
| (MDC) | COACTIVATOR-3 (NCoA-3) | (TYPE 1) |
| METASTASIN (MTS-1) | NUCLEOSIDE | PLASMINOGEN |
| MLN64 | DIPHOSPHATE KINASE A | ACTIVATOR INHIBITOR |
| MMP1 | (NDPKA) | (TYPE 2) |
| MMP2 | NUCLEOSIDE | PLASMINOGEN |
| MMP3 | DIPHOSPHATE KINASE B | ACTIVATOR (TISSUE- |
| MMP7 | (NDPKB) | TYPE) |
| MMP9 | ONCOSTATIN M (OSM) | PLASMINOGEN |
| MMP11 | ORNITHINE | ACTIVATOR (UROKINASE- |
| MMP13 | DECARBOXYLASE (ODC) | TYPE) |
| MMP14 | OSTEOCLAST | PLATELET |
| MMP15 | DIFFERENTIATION | GLYCOPROTEIN IIIa |
| MMP16 | FACTOR (ODF) | (GP3A) |
| MMP17 | OSTEOCLAST | PLAU |
| MOESIN | DIFFERENTIATION | PLEOMORPHIC ADENOMA |
| MONOCYTE ARGININE- | FACTOR RECEPTOR | GENE-LIKE 1 (PLAGL1) |
| SERPIN | (ODFR) | POLYMORPHIC |
| MONOCYTE-DERIVED | OSTEONECTIN (OSN, ON) | EPITHELIAL MUCIN (PEM) |
| NEUTROPHIL | OSTEOPONTIN (OPN) | PRAD1 |
| CHEMOTACTIC FACTOR | OXYTOCIN RECEPTOR | PROGESTERONE |
| MONOCYTE-DERIVED | (OXTR) | RECEPTOR (PgR) |
| PLASMINOGEN | p27/kip1 | PROGESTERONE |
| ACTIVATOR INHIBITOR | p300/CBP COINTEGRATOR | RESISTANCE |
| MTS-1 | ASSOCIATE PROTEIN | PROSTAGLANDIN |
| MUC-1 | (p/CIP) | ENDOPEROXIDE |
| MUC18 | p53 | SYNTHASE-2 |
| MUCIN LIKE CANCER | p9Ka | PROSTAGLANDIN G/H |
| ASSOCIATED ANTIGEN | PAI-1 | SYNTHASE-2 |
| (MCA) | THYROID HORMONE | VASCULAR PERMEABILITY |
| MUCIN | RECEPTOR ACTIVATOR | FACTOR |
| MUC-1 | MOLECULE 1 (TRAM-1) | VEGFR2 |
| PROSTAGLANDIN H | TIGHT JUNCTION PROTEIN | VERY LATE T-CELL |
| SYNTHASE-2 | 1 (TJP1) | ANTIGEN BETA (VLA-BETA) |
| pS2 | TIMP1 | VIMENTIN |
| PS6K | TIMP2 | VITRONECTIN RECEPTOR |
| PSORIASIN | TIMP3 | ALPHA POLYPEPTIDE |
| PTHLH | TIMP4 | (VNRA) |
| PTHrP | TISSUE-TYPE | VITRONECTIN RECEPTOR |
| RAD51 | PLASMINOGEN | VON WILLEBRAND |
| RAD52 | ACTIVATOR | FACTOR |
| RAD54 | TN-C | VPF |
| RAP46 | TP53 | VWF |
| RECEPTOR-ASSOCIATED | tPA | WNT-1 |
| COACTIVATOR 3 (RAC3) | TRANSCRIPTIONAL | ZAC |
| REPRESSOR OF | INTERMEDIARY FACTOR 2 | ZO-1 |
| ESTROGEN RECEPTOR | (TIF2) | ZONULA OCCLUDENS-1 |
| ACTIVITY (REA) | TREFOIL FACTOR 1 (TFF1) | |
| S100A4 | TSG101 | |
| S100A6 | TSP-1 | |
| S100A7 | TSP1 | |
| S6K | TSP-2 | |
| SART-1 | TSP2 | |
| SCAFFOLD ATTACHMENT | TSP50 | |
| FACTOR B (SAF-B) | TUMOR CELL | |
| SCATTER FACTOR (SF) | COLLAGENASE | |
| SECRETED | STIMULATING FACTOR | |
| PHOSPHOPROTEIN-1 | (TCSF) | |
| (SPP-1) | TUMOR-ASSOCIATED | |
| SECRETED PROTEIN | EPITHELIAL MUCIN | |
| ACIDIC AND RICH IN | uPA | |
| CYSTEINE (SPARC) | uPAR | |
| STANNICALCIN | UROKINASE | |
| STEROID RECEPTOR | UROKINASE-TYPE | |
| COACTIVATOR-1 (SRC-1) | PLASMINOGEN | |
| STEROID RECEPTOR | ACTIVATOR | |
| COACTIVATOR-2 (SRC-2) | UROKINASE-TYPE | |
| STEROID RECEPTOR | PLASMINOGEN | |
| COACTIVATOR-3 (SRC-3) | ACTIVATOR RECEPTOR | |
| STEROID RECEPTOR RNA | (uPAR) | |
| ACTIVATOR (SRA) | UVOMORULIN | |
| STROMELYSIN-1 | VASCULAR ENDOTHELIAL | |
| STROMELYSIN-3 | GROWTH FACTOR | |
| TENASCIN-C (TN-C) | VASCULAR ENDOTHELIAL | |
| TESTES-SPECIFIC | GROWTH FACTOR | |
| PROTEASE 50 | RECEPTOR-2 (VEGFR2) | |
| THROMBOSPONDIN I | VASCULAR ENDOTHELIAL | |
| THROMBOSPONDIN II | GROWTH FACTOR-A | |
| THYMIDINE | | |
| PHOSPHORYLASE (TP) | | |

Any sample mobilization component may be used in the device. For example, the sample mobilizer may include a mechanical rocker or a sonicator. Alternatively, it may be adapted to provide centrifugal force to the receptacle and lid. A centrifugal sample mobilizer may be used to mobilize sample components, e.g., cells, within a fluid sample, e.g., a fluid sample having a free surface. A centrifugal sample mobilizer may also be used to drive cell rolling along the lid surface. In one example, a centrifugal sample mobilizer may include an axle that rotates the receptacle; in some embodiments, the centrifugal force generated by operating the device is capable of driving the lid into a nonorthogonal angle with respect to the axle.

Another sample mobilization component that may be used in the device utilizes two fluidically coupled chambers, each of which has a surface in contact with the internal space of the receptacle. In such a device, which utilizes pressure-driven flow, each chamber is filled with a fluid, e.g., air, and when one chamber is compressed, a portion of the fluid therein enters the other chamber, increasing its volume. By placing these chambers in contact with a cellular sample in the receptacle and altering their volumes, e.g., squeezing the chambers in alternation, the sample is mobilized.

Uses of Devices of the Invention

The invention features improved devices for the enrichment of CTCs and other particles, including bacteria, viruses, fungi, cells, cellular components, viruses, nucleic acids, proteins, and protein complexes, according to size. The devices may be used to effect various manipulations on particles in a sample. Such manipulations include enrichment or concentration of a particle, including size based fractionation, or alteration of the particle itself or the fluid carrying the particle. Preferably, the devices are employed to enrich CTCs or other rare particles from a heterogeneous mixture or to alter a rare particle, e.g., by exchanging the liquid in the suspension or by contacting a particle with a reagent. Such devices allow for a high degree of enrichment with limited stress on cells, e.g., reduced mechanical lysis or intracellular activation of cells.

Array Design

Single-stage array. In one embodiment, a single stage contains an array of obstacles, e.g., cylindrical obstacles (FIG. 1D), forming a network of gaps. In certain embodiments, the array has a maximum pass-through size that is several times larger than the cut-off size, e.g., when enriching CTCs from other cells in a blood sample. This result may be achieved using a combination of a large gap size d and a small bifurcation ratio ε. In preferred embodiments, the ε is at most 1/2, e.g., at most 1/3, 1/10, 1/30, 1/100, 1/300, or 1/1000. In such embodiments, the obstacle shape may affect the flow profile in the gap, e.g., such that fluid flowing through the gaps is unevenly distributed around the obstacles; however, the obstacles may be compressed in the flow direction, in order to make the array short (FIG. 1E). Single stage arrays may include bypass channels as described herein.

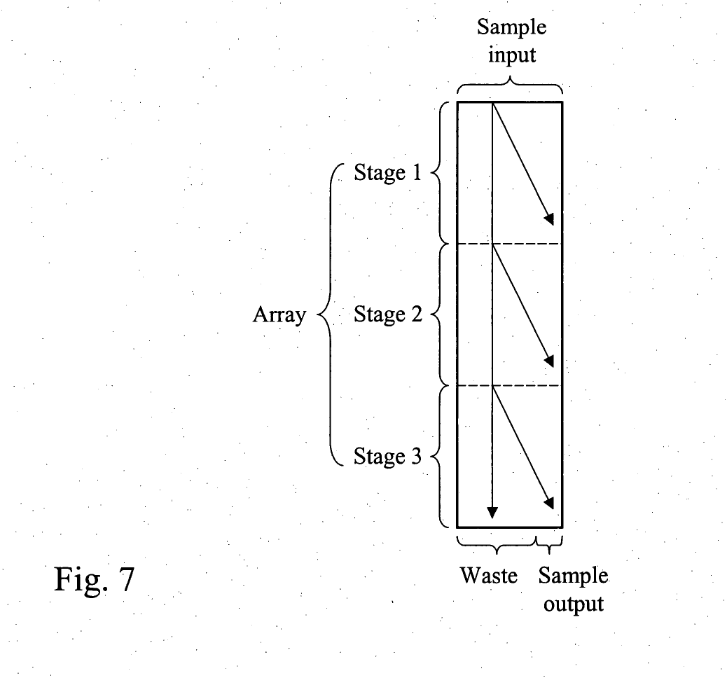

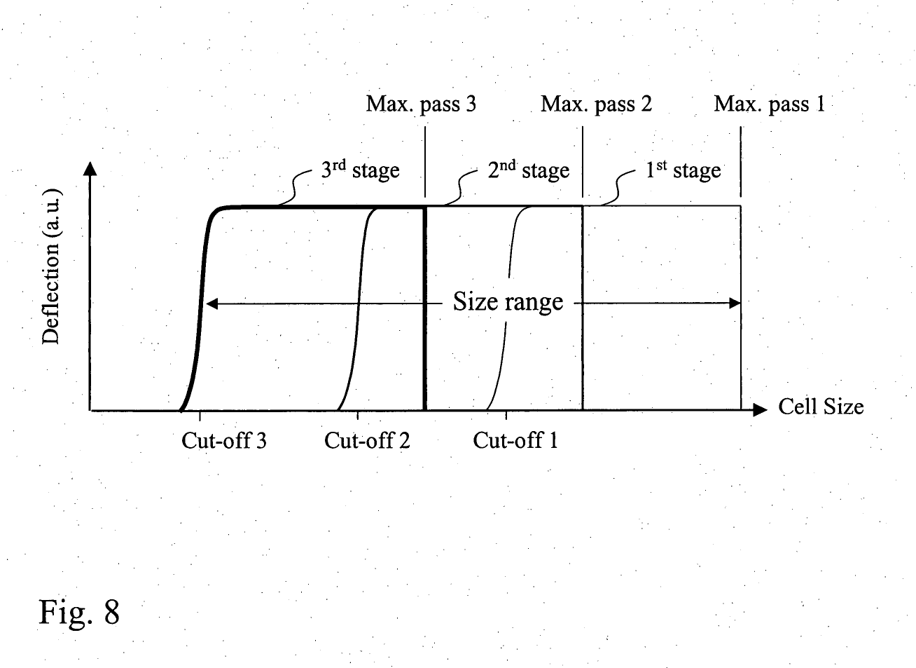

Multiple-stage arrays. In another embodiment, multiple stages are employed to enrich particles over a wide size range. An exemplary device is shown in FIG. 7. The device shown has three stages, but any number of stages may be employed. Typically, the cut-off size in the first stage is larger than the cut-off in the second stage, and the first stage cut-off size is smaller than the maximum pass-through size of the second stage (FIG. 8). The same is true for the following stages. The first stage will deflect (and remove) particles, e.g., that would cause clogging in the second stage, before they reach the second stage. Similarly, the second stage will deflect (and remove) particles that would cause clogging in the third stage, before they reach the third stage. In general, an array may have as many stages as desired, connected either serially or in parallel.

As described, in a multiple-stage array, large particles, e.g., cells, that could cause clogging downstream are deflected first, and these deflected particles need to bypass the downstream stages to avoid clogging. Thus, devices of the invention may include bypass channels that remove output from an array. Although described here in terms of removing particles above the critical size, bypass channels may also be employed to remove output from any portion of the array.

Different designs for bypass channels are as follows.

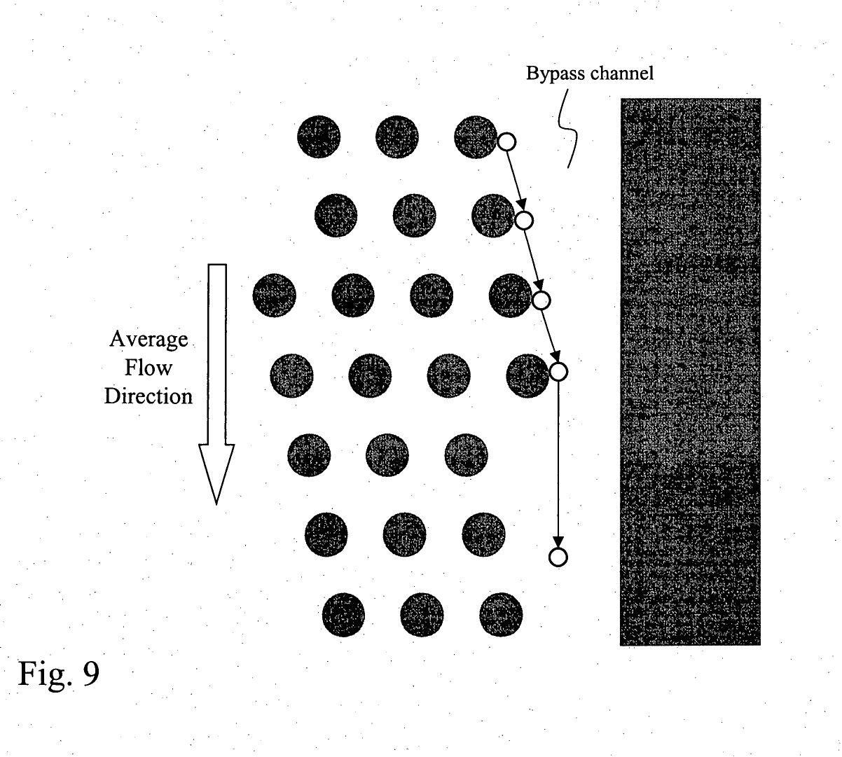

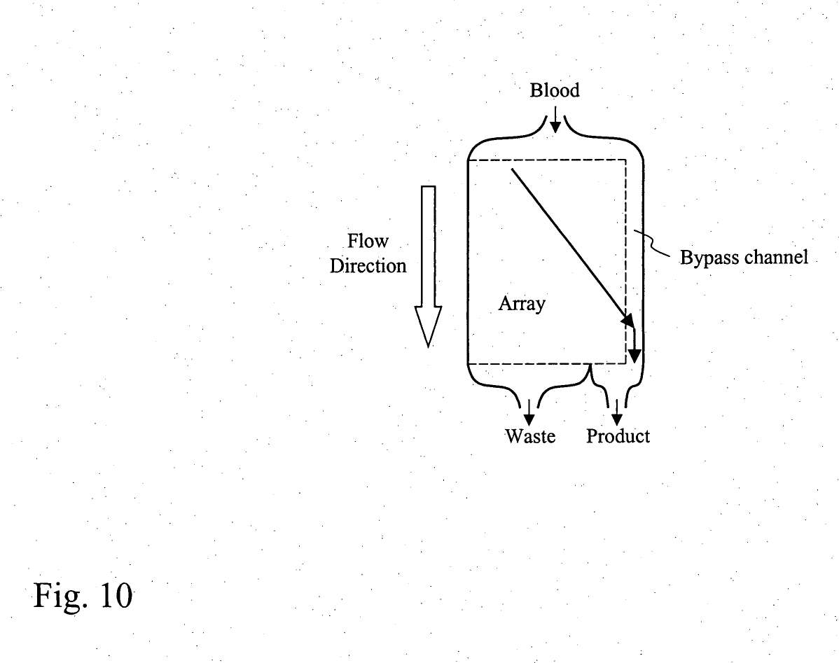

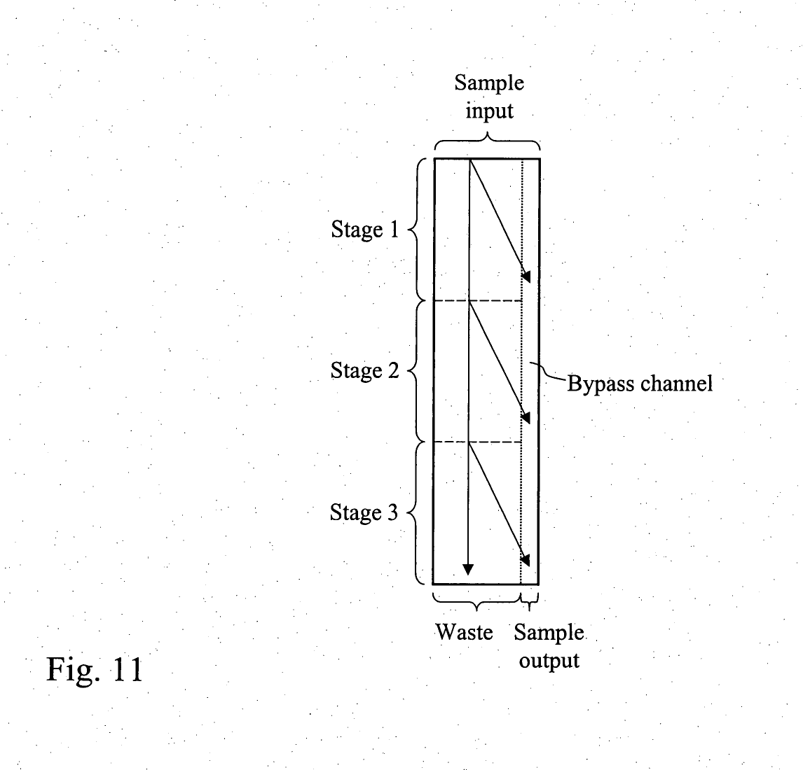

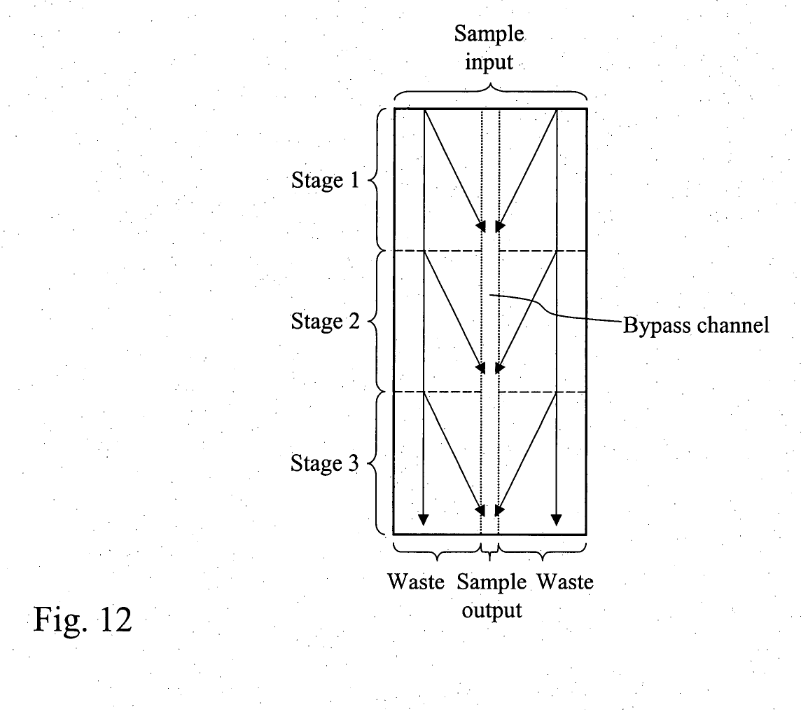

Single bypass channels. In this design, all stages share one bypass channel, or there is only one stage. The physical boundary of the bypass channel may be defined by the array boundary on one side and a sidewall on the other (FIGS. 9-11). Single bypass channels may also be employed with duplex arrays (FIG. 12).

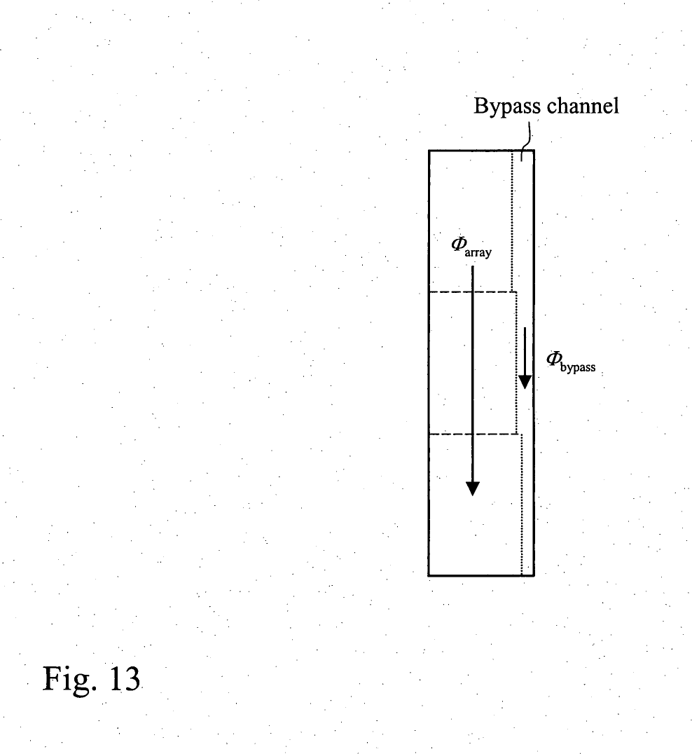

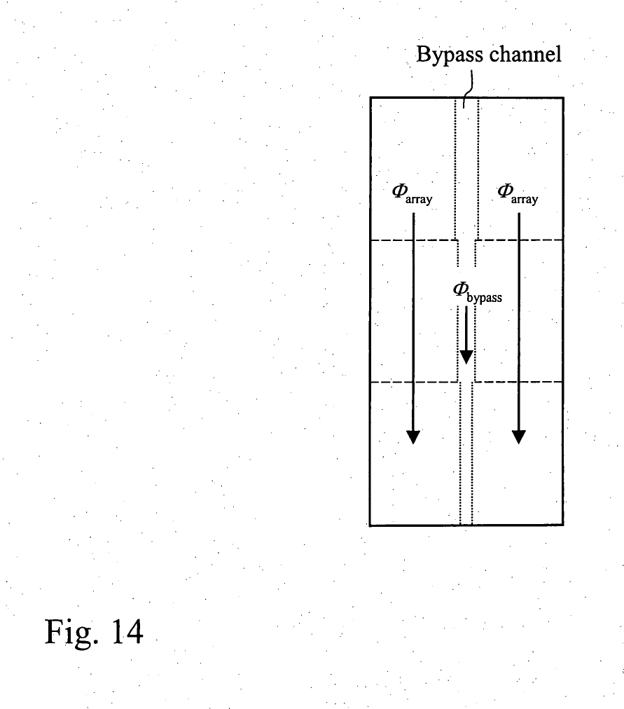

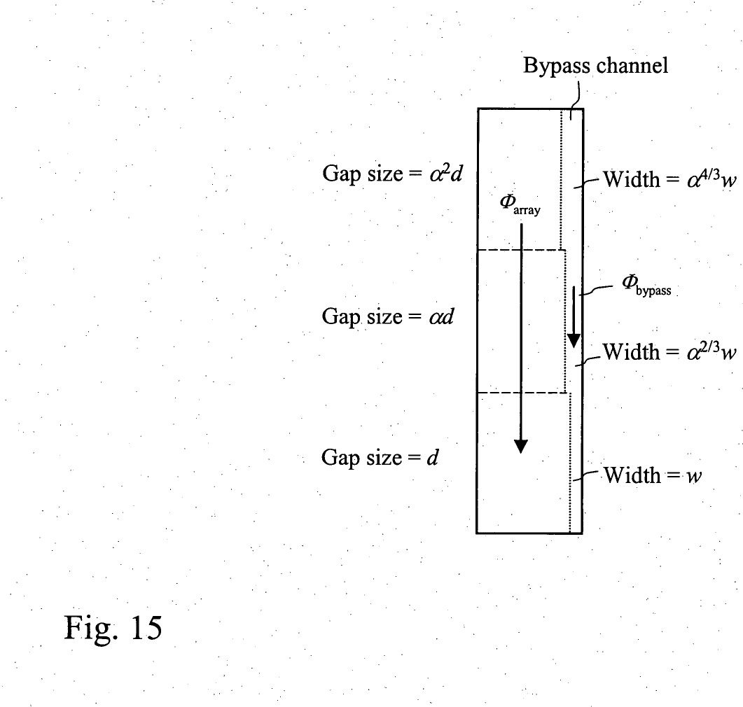

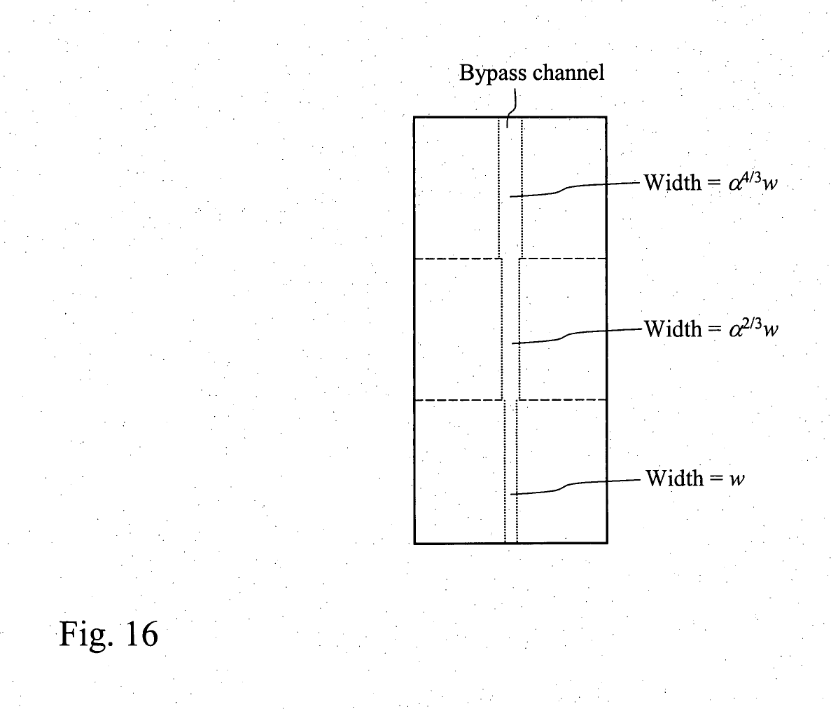

Single bypass channels may also be designed, in conjunction with an array to maintain constant flux through a device (FIG. 13). The bypass channel has varying width designed maintain constant flux through all the stages, so that the flow in the channel does not interfere with the flow in the arrays. Such a design may also be employed with an array duplex (FIG. 14). Single bypass channels may also be designed in conjunction with the array in order to maintain substantially constant fluidic resistance through all stages (FIG. 15). Such a design may also be employed with an array duplex (FIG. 16.)

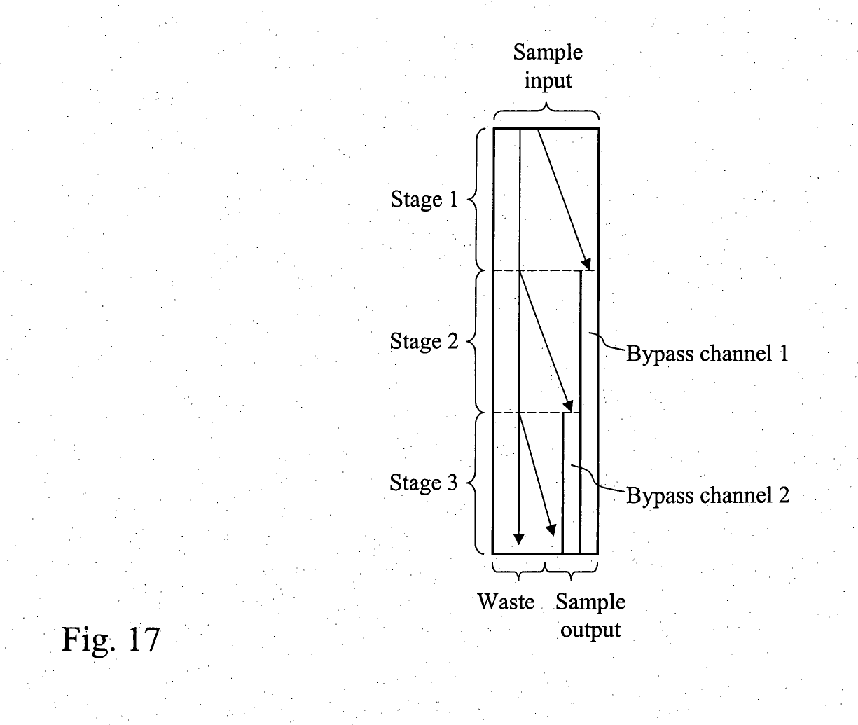

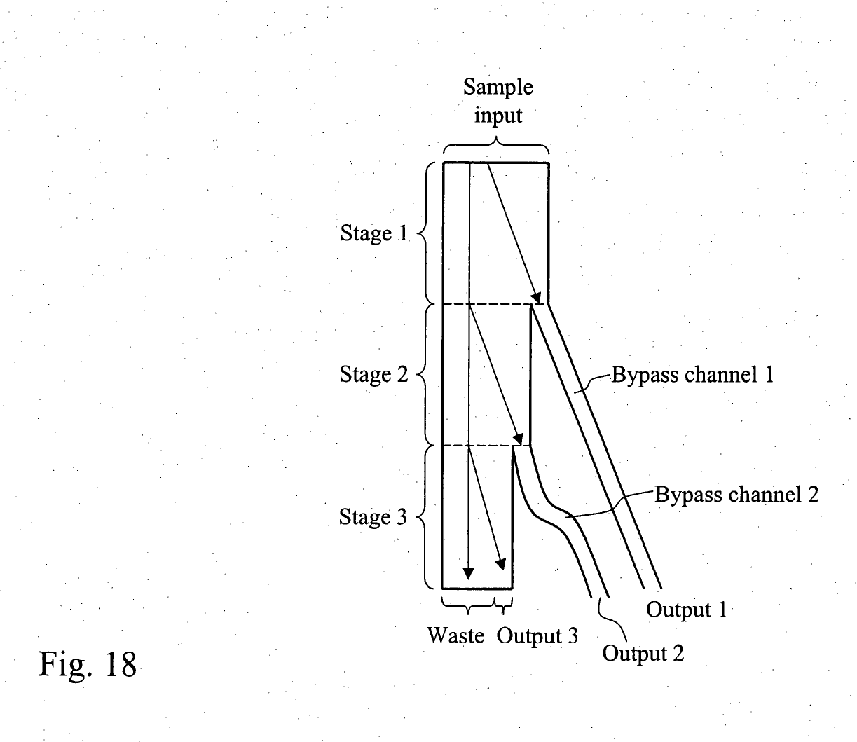

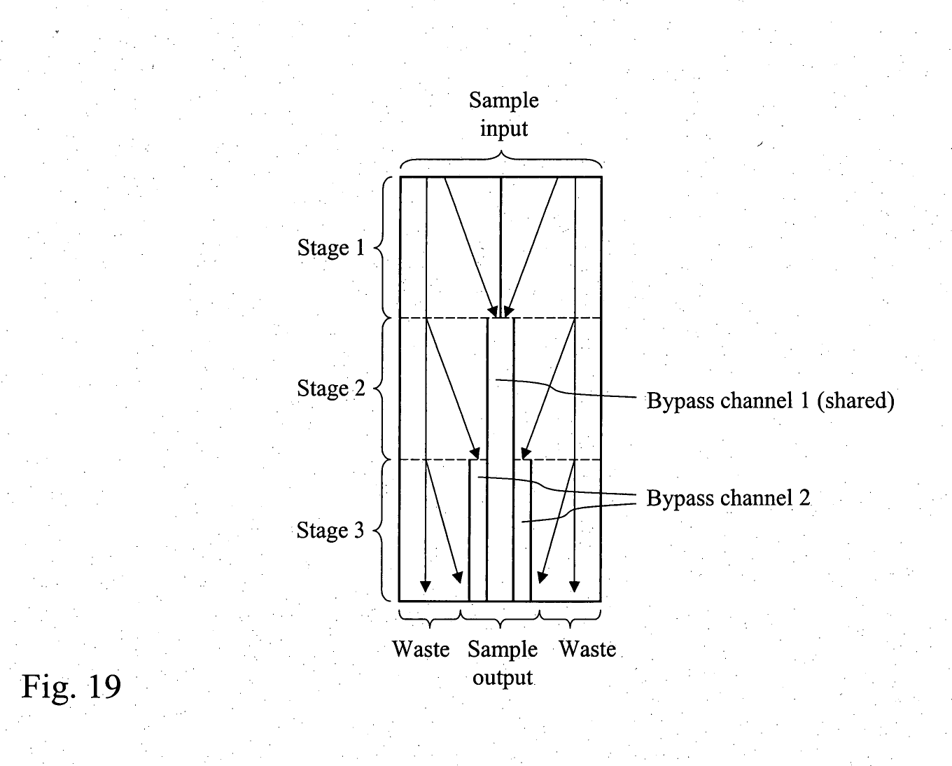

Multiple bypass channels. In this design (FIG. 17), each stage has its own bypass channel, and the channels are separated from each other by sidewalls. Large particles, e.g., cells are deflected into the major flux to the lower right corner of the first stage and then into in the bypass channel (bypass channel 1 in FIG. 17). Smaller cells that would not cause clogging in the second stage proceed to the second stage, and cells above the critical size of the second stage are deflected to the lower right corner of the second stage and into in another bypass channel (bypass channel 2 in FIG. 17). This design may be repeated for as many stages as desired. In this embodiment, the bypass channels are not fluidically connected, allowing for collection or other manipulation of multiple fractions. The bypass channels do not need to be straight or be physically parallel to each other (FIG. 18). Multiple bypass channels may also be employed with duplex arrays (FIG. 19).

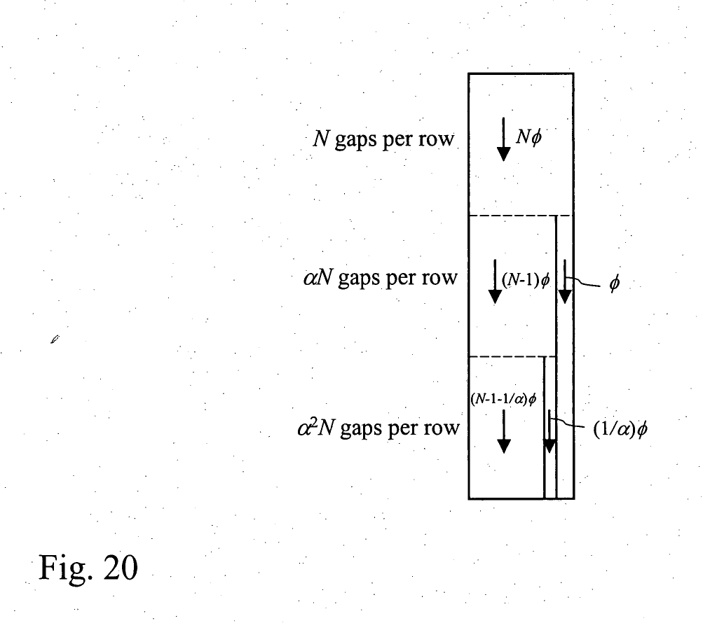

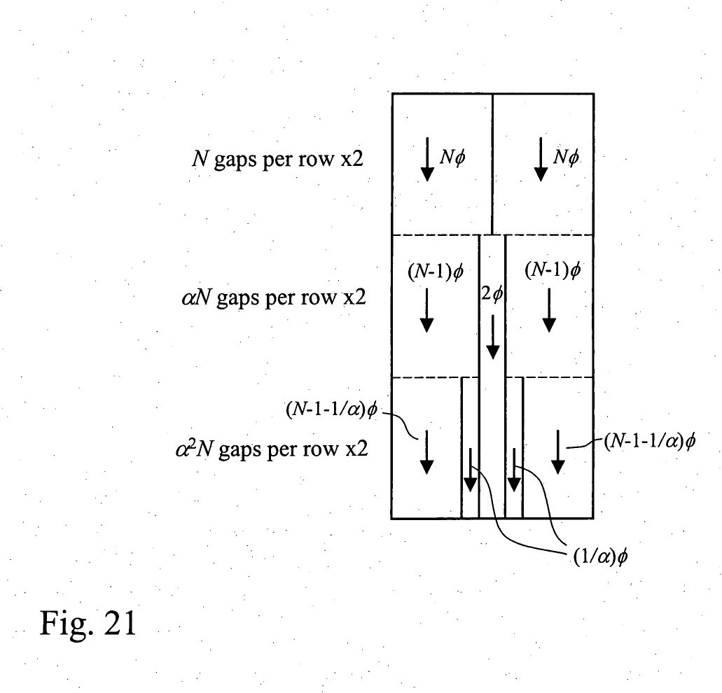

Multiple bypass channels may be designed, in conjunction with an array to maintain constant flux through a device (FIG. 20). In this example, bypass channels are designed to remove an amount of flow so the flow in the array is not perturbed, i.e., substantially constant. Such a design may also be employed with an array duplex (FIG. 21). In this design, the center bypass channel may be shared between the two arrays in the duplex.

Optimal Boundary Design. If the array were infinitely large, the flow distribution would be the same at every gap. The flux φ going through a gap would be the same, and the minor flux would be εφ for every gap. In practice, the boundaries of the array perturb this infinite flow pattern. Portions of the boundaries of arrays may be designed to generate the flow pattern of an infinite array. Boundaries may be flow-feeding, i.e., the boundary injects fluid into the array or flow-extracting, i.e., the boundary extracts fluid from the array.

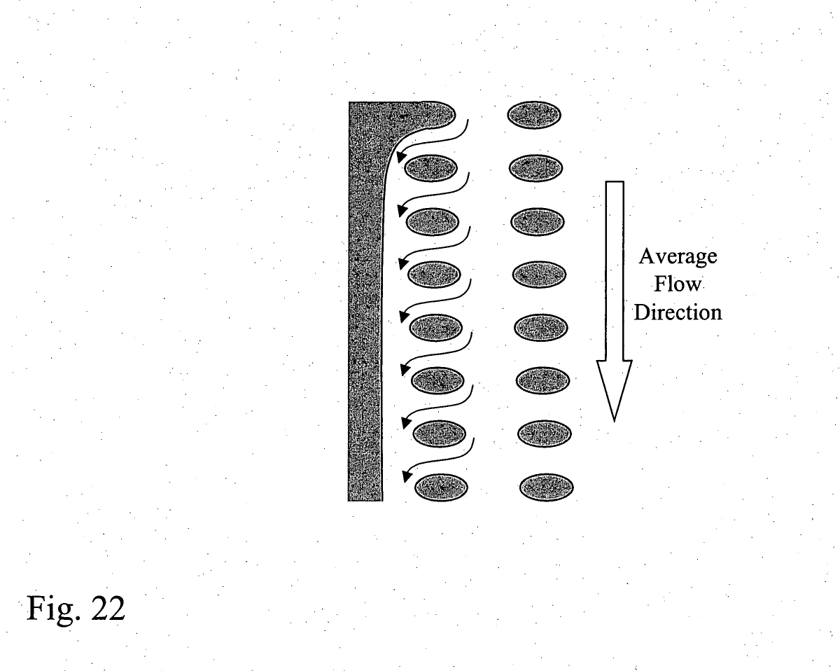

A preferred flow-extracting boundary widens gradually to extract εφ (represented by arrows in FIG. 22) from each gap at the boundary (d=24 μm, ε=1/60). For example, the distance between the array and the sidewall gradually increases to allow for the addition of εφ from each gap to the boundary. The flow pattern inside this array is not affected by the bypass channel because of the boundary design.

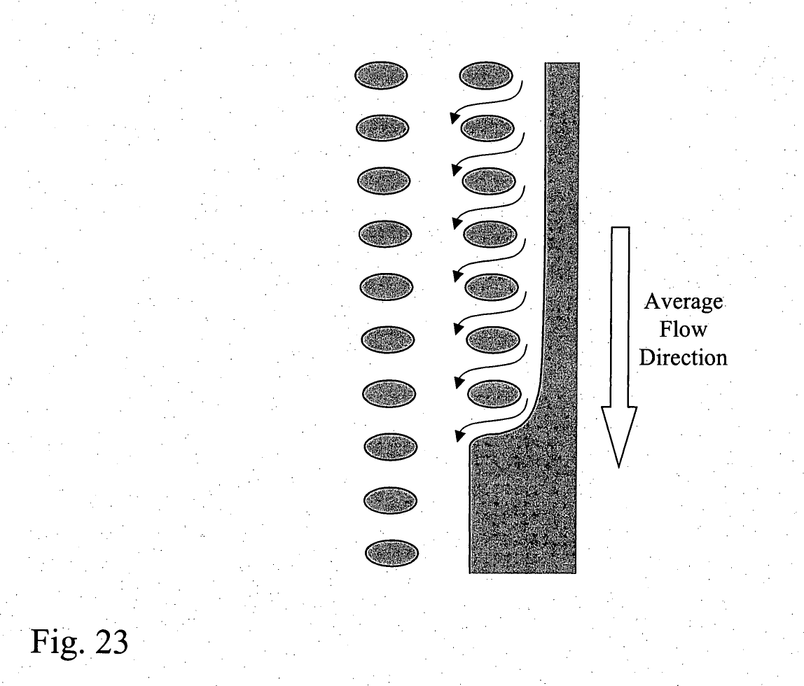

A preferred flow-feeding boundary narrows gradually to feed exactly εφ (represented by arrows in FIG. 23) into each gap at the boundary (d=24 μm, ε=1/60). For example, the distance between the array and the sidewall gradually decreases to allow for the removal of εφ to each gap from the boundary. Again, the flow pattern inside this array is not affected by the bypass channel because of the boundary design.

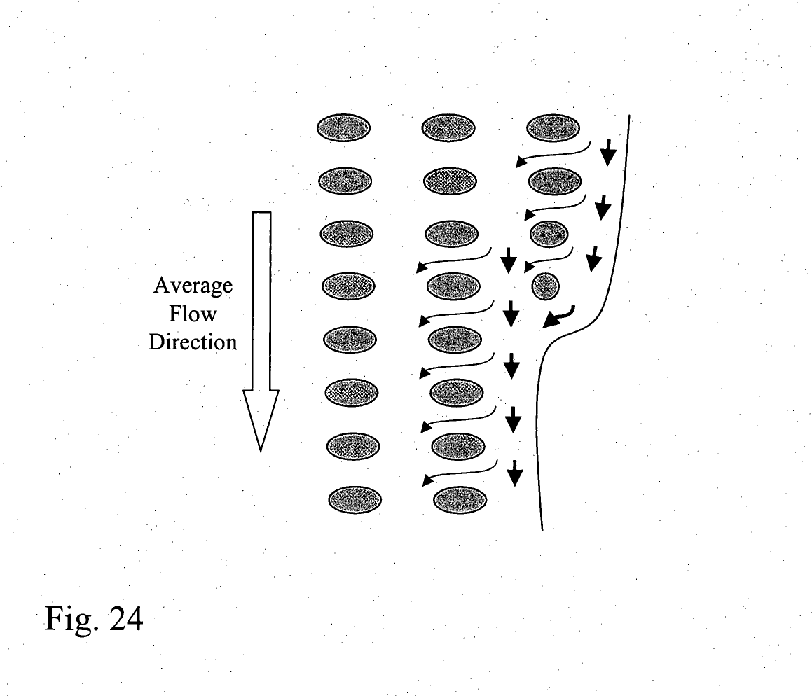

A flow-feeding boundary may also be as wide as or wider than the gaps of an array (FIG. 24) (d=24 μm, ε=1/60). A wide boundary may be desired if the boundary serves as a bypass channel, e.g., to allow for collection of particles. A boundary may be employed that uses part of its entire flow to feed the array and feeds εφ into each gap at the boundary (represented by arrows in FIG. 24).

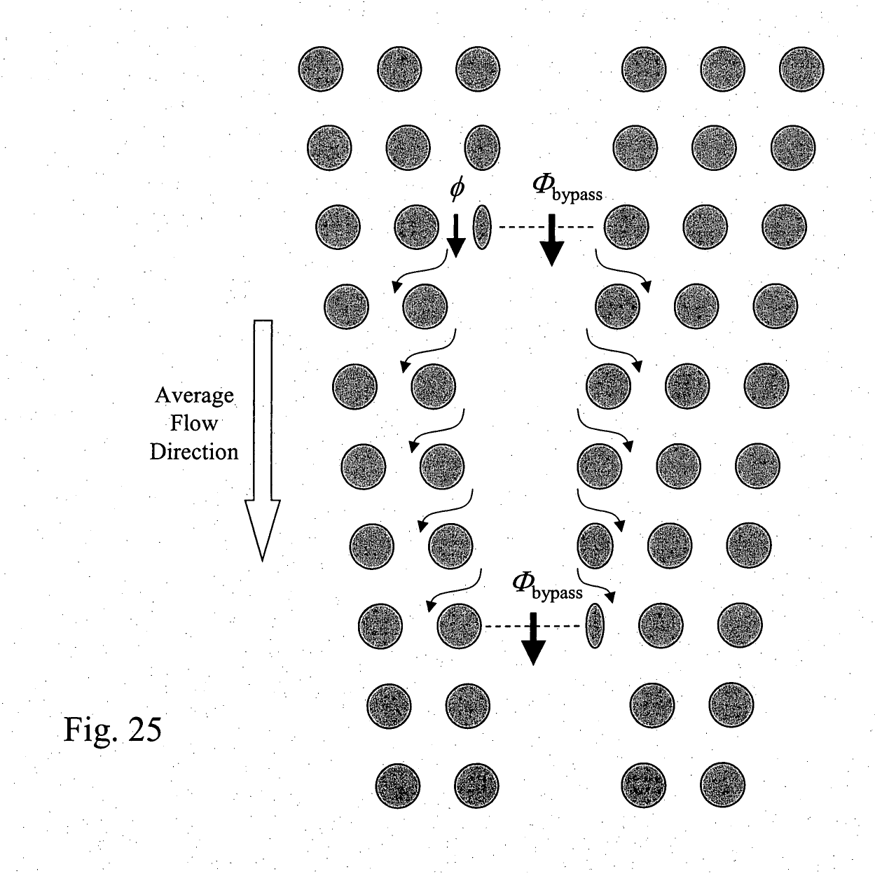

FIG. 25 shows a single bypass channel in a duplex array (ε=1/10, d=8 μm). The bypass channel includes two flow-feeding boundaries. The flux across the dashed line 1 in the bypass channel is Φ_{bypass}. A flow φ joins Φ_{bypass} from a gap to the left of the dashed line. The shapes of the obstacles at the boundaries are adjusted so that the flows going into the arrays are εφ at each gap at the boundaries. The flux at dashed line 2 is again Φ_{bypass}.

In some cases, arrays of the invention may include a plurality of rows of obstacles, each successive row being offset by less than half of the period of the previous row, such that at least 50%, 60%, 70%, 80%, 90%, 95%, or even 99% of gaps between obstacles each has a length approximately equal to a first length parameter, and at most 50%, 40%, 30%, 20%, 10%, 5%, or even 1%, respectively, of gaps between obstacles each has a length approximately equal to a second length parameter shorter than the first length parameter. Gaps having a length approximately equal to the second length parameter may be distributed throughout the array either uniformly or non-uniformly. The second length parameter may be sized to capture a cell of interest larger than a predetermined size from a cellular sample. The first length parameter is longer than the second length parameter, e.g., by a factor of 1.1, 1.5, 2, 3, 5, 10, 20, 50, or even 100. Exemplary distances for the first length parameter are in the range of 30 to 100 microns, and exemplary distances for the second length parameter are in the range of 10 to 50 microns.

Optionally, each obstacle of an array of the invention has approximately the same size; alternatively, at least 50%, 60%, 70%, 80%, 90%, 95%, or even 99% of the obstacles have approximately the same size. In some cases, at least 50%, 60%, 70%, 80%, 90%, 95%, or even 99% of the gaps between obstacles in each row each has a length approximately equal to a first length parameter, and up to 50%, 40%, 30%, 20%, 10%, 5%, or even 1%, respectively, of the gaps between obstacles in each row each has a length approximately equal to a second length parameter, which may be shorter than the first length parameter.

In some arrays, a subset of the obstacles, e.g., 50%, 40%, 30%, 20%, 10%, 5%, or even 1%, are unaligned with the centers of the remaining obstacles in their row. Unaligned obstacles may be distributed throughout the array either uniformly or non-uniformly.

Arrays of the invention may have obstacles with different cross-sections; for example, 50%, 60%, 70%, 80%, 90%, 95%, or even 99% of the obstacles may each have a cross-sectional area approximately equal to a first area parameter, and 50%, 40%, 30%, 20%, 10%, 5%, or even 1%, respectively, of the obstacles may each have a cross-sectional area approximately equal to a second area parameter. Optionally, the second area parameter is larger than the first area parameter. In addition, at least one obstacle having a cross-sectional area approximately equal to the first area parameter or second area parameter may have an asymmetrical cross-section.

Arrays of the invention may also include a first subarray of obstacles and a second subarray of obstacles, such that each of the subarrays includes a gap between two obstacles in that subarray, and such that the array includes an interface between the first subarray and the second subarray including a restricted gap that is smaller than the gap between two obstacles in either subarray. The subarrays may be arranged in a two-dimensional configuration; furthermore, they may be staggered, either periodically or uniformly. Each subarray may contain any number of obstacles, e.g., between 2 and 200, between 3 and 50, or between 6 and 20. Exemplary diameters for subarray obstacles are, e.g., in the range of 25 to 200 microns. In general, the gap between two obstacles in an array of the invention may be, e.g., at least 20, 40, 60, 80, or 100 microns; in the case of the restricted gap described above, this gap may be, e.g., at most 100, 80, 60, 40, or 20 microns. Other gap lengths are also possible.

Arrays of the invention may be coupled to a substrate, e.g., plastic, and may include a microfluidic gap. Arrays may additionally be coupled to one or more binding moieties, e.g., binding moieties described herein, that selectively bind to cells of interest. Arrays may also be inside a receptacle, e.g., a receptacle coupled to a transparent cover.

In another embodiment, a two-dimensional array of obstacles forms a network of gaps, such that the array of obstacles includes a plurality of rows distributed on a surface to create fluid flow paths through the device, wherein at least 50%, 60%, 70%, 80%, 90%, 95%, or even 99% of the flow paths each has a width approximately equal to a first width parameter, and at most 50%, 40%, 30%, 20%, 10%, 5%, or even 1%, respectively, of the flow paths each has a width approximately equal to a second, smaller width parameter. Such an array may be used, e.g., to enrich an analyte from a fluid sample. Flow paths each having a width approximately equal to the second width parameter may be distributed throughout the device either uniformly or non-uniformly, and the second width parameter may be sized to capture the desired analyte within the flow paths that are approximately of the second width parameter. Optionally, the array includes an inlet and an outlet. Optionally, in arrays that include outlets, a region of obstacles with flow path widths equal to or smaller than the second width surrounds the outlet. Such devices may, e.g., have three two-dimensional arrays fluidly connected in series, such that the percentage of the flow paths of the second width increases in the direction of flow of fluid through the device.

Arrays may be coupled to other elements to form devices of the invention. For example, an array may be fluidically coupled to a sample reservoir, a detector, or other elements or modules disclosed herein. Arrays may also function as devices without the need for additional elements or modules. In addition, arrays of the invention may be two-dimensional arrays, or they may adopt another geometry.

Any of the arrays described herein may be used in conjunction with any of the devices or methods of the invention.

Device Design

On-Chip Flow Resistor for Defining and Stabilizing Flow

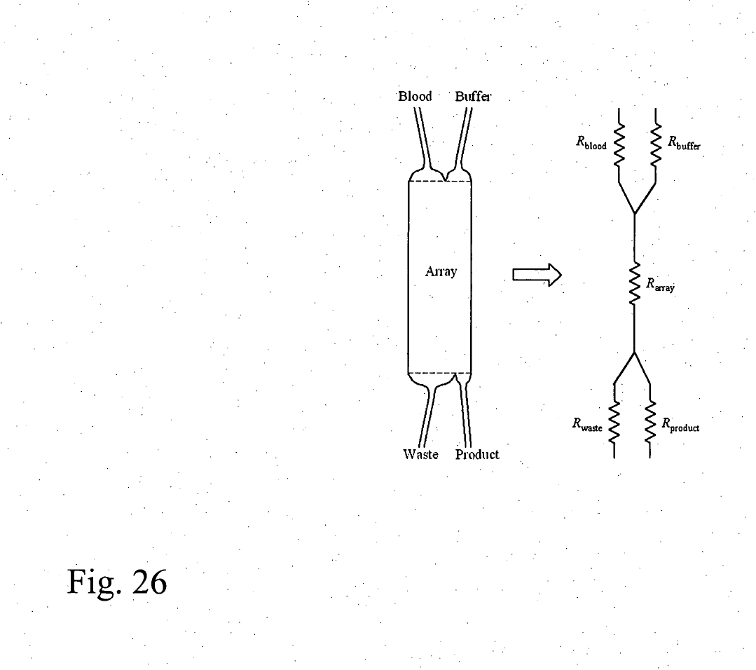

Devices of the invention may also employ fluidic resistors to define and stabilize flows within an array and to also define the flows collected from the array. FIG. 26 shows a schematic of planar device; a sample, e.g., blood containing CTCs, inlet channel, a buffer inlet channel, a waste outlet channel, and a product outlet channel are each connected to an array. The inlets and outlets act as flow resistors. FIG. 26 also shows the corresponding fluidic resistances of these different device components.

Flow Definition within the Array

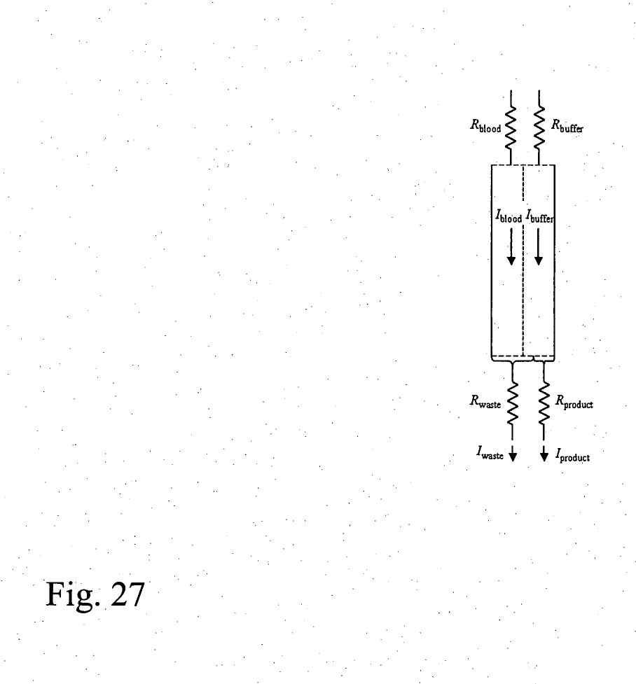

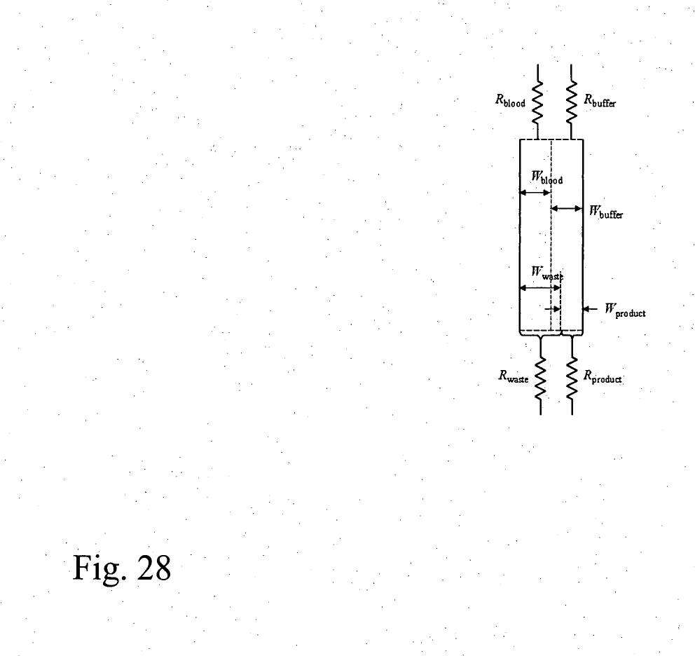

FIGS. 27 and 28 show the currents and corresponding widths of the sample and buffer flows within the array when the device has a constant depth and is operated with a given pressure drop. The flow is determined by the pressure drop divided by the resistance. In this particular device, I_{blood} and I_{buffer} are equivalent, and this determines equivalent widths of the blood and buffer streams in the array.

Definition of Collection Fraction

By controlling the relative resistance of the product and waste outlet channels, one may modulate the collection tolerance for each fraction. For example, in this particular set of schematics, when R_{product} is greater than R_{waste}, a more concentrated product fraction will result at the expense of a potentially increased loss to and dilution of waste fraction. Conversely, when R_{product} is less than R_{waste}, a more dilute and higher yield product fraction will be collected at the expense of potential contamination from the waste stream.

Multiplexed Arrays

The invention features multiplexed arrays. Putting multiple arrays on one device increases sample-processing throughput of CTCs or other cells of interest and allows for parallel processing of multiple samples or portions of the sample for different fractions or manipulations. Multiplexing is further desirable for preparative devices. The simplest multiplex device includes two devices attached in series, i.e., a cascade. For example, the output from the major flux of one device may be coupled to the input of a second device. Alternatively, the output from the minor flux of one device may be coupled to the input of the second device.

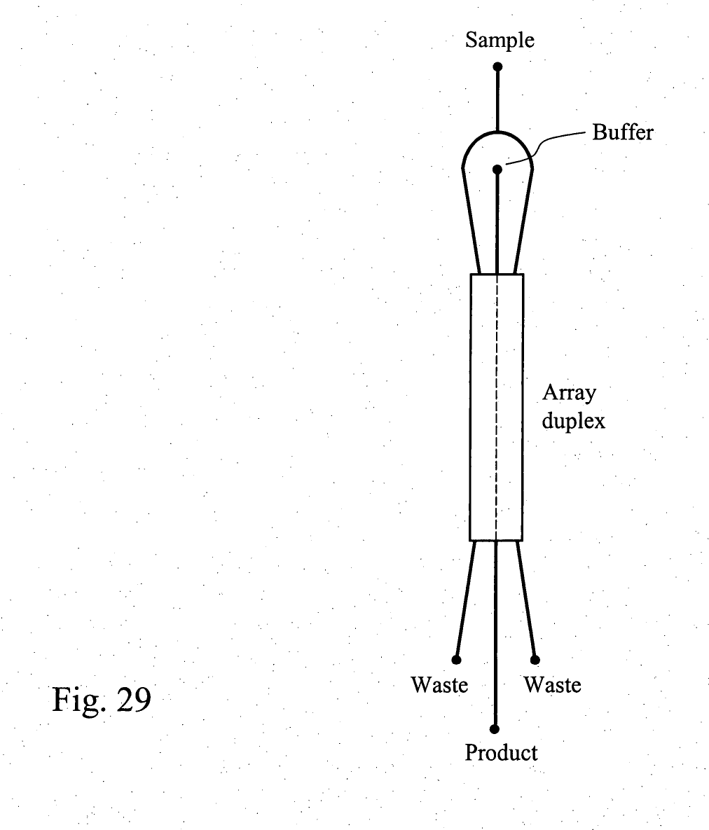

Duplexing. Two arrays may be disposed side-by-side, e.g., as mirror images (FIG. 29). In such an arrangement, the critical size of the two arrays may be the same or different. Moreover, the arrays may be arranged so that the major flux flows to the boundary of the two arrays, to the edge of each array, or a combination thereof. Such a multiplexed array may also contain a central region disposed between the arrays, e.g., to collect particles above the critical size or to alter the sample, e.g., through buffer exchange, reaction, or labeling.

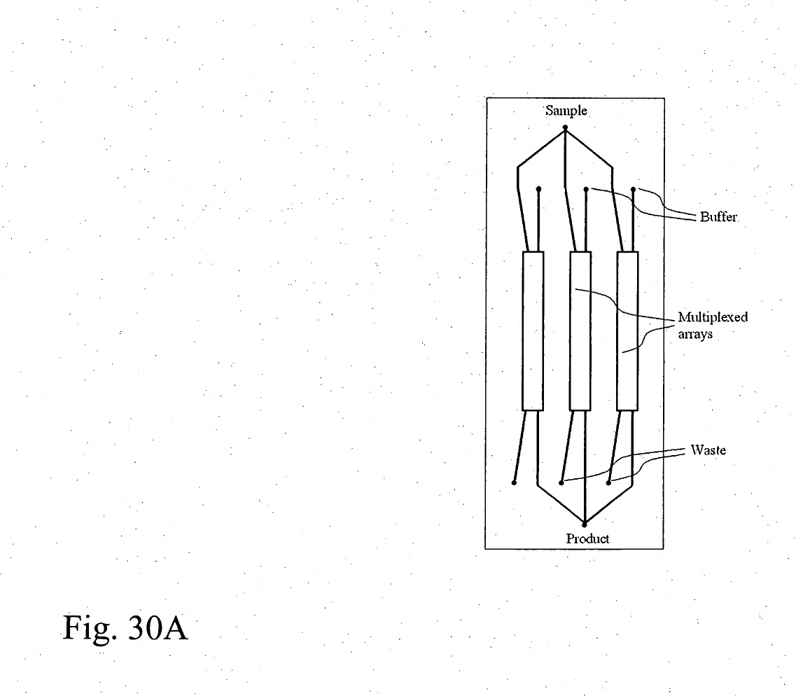

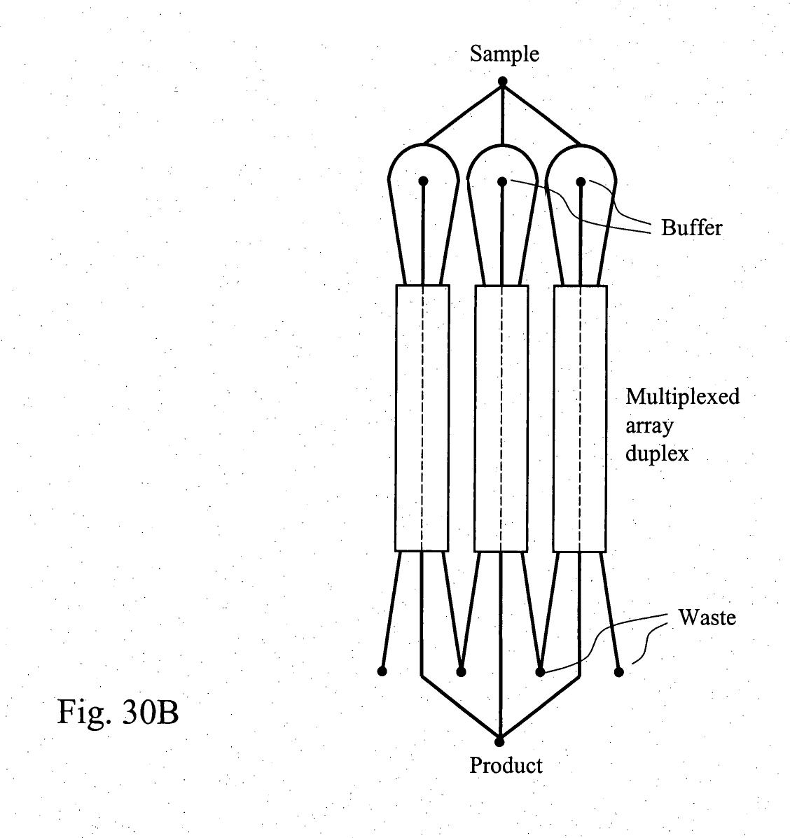

Multiplexing on a device. In addition to forming a duplex, two or more arrays that have separated inputs may be disposed on the same device (FIG. 30A). Such an arrangement could be employed for multiple samples, or the plurality of arrays may be connected to the same inlet for parallel processing of the same sample. In parallel processing of the same sample, the outlets may or may not be fluidically connected. For example, when the plurality of arrays has the same critical size, the outlets may be connected for high throughput samples processing. In another example, the arrays may not all have the same critical size or the particles in the arrays may not all be treated in the same manner, and the outlets may not be fluidically connected.

Multiplexing may also be achieved by placing a plurality of duplex arrays on a single device (FIG. 30B). A plurality of arrays, duplex or single, may be placed in any possible three-dimensional relationship to one another.

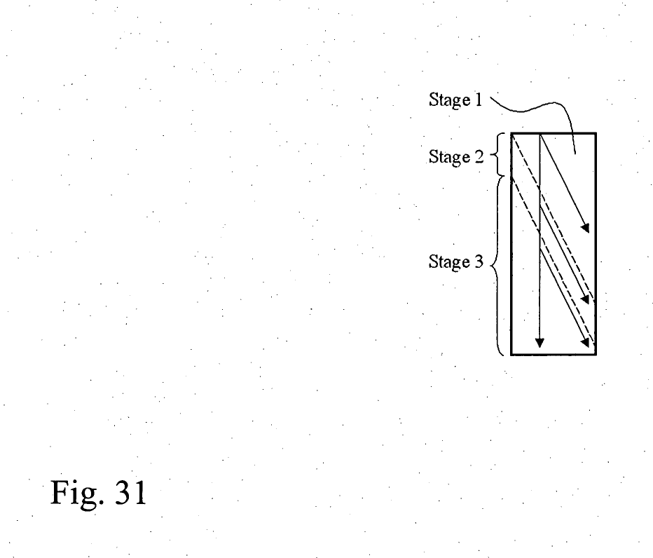

Devices of the invention also feature a small footprint. Reducing the footprint of an array may lower cost, and reduce the number of collisions with obstacles to eliminate any potential mechanical damage or other effects to particles. The length of a multiple stage array may be reduced if the boundaries between stages are not perpendicular to the direction of flow. The length reduction becomes significant as the number of stages increases. FIG. 31 shows a small-footprint three-stage array.

Additional Components

In addition to an array of gaps, devices of the invention may include additional elements or modules, e.g., for isolation, enrichment, collection, manipulation, or detection, e.g., of CTCs. Such elements are known in the art. For example, devices may include one or more inlets for sample or buffer input, and one or more outlets for sample output. Arrays may also be employed on a device having components for other types of enrichment or other manipulation, including affinity, magnetic, electrophoretic, centrifugal, and dielectrophoretic enrichment. Devices of the invention may also be employed with a component for two-dimensional imaging of the output from the device, e.g., an array of wells or a planar surface. Preferably, arrays of gaps as described herein are employed in conjunction with an affinity enrichment.

In one example, a detection module is fluidically coupled to a separation or enrichment device of the invention. The detection module may operate using any method of detection disclosed herein, or other methods known in the art. For example, the detection module includes a microscope, a cell counter, a magnet, a biocavity laser (see, e.g., Gourley et al., J. Phys. D: Appl. Phys. 36: R228-R239 (2003)), a mass spectrometer, a PCR device, an RT-PCR device, a matrix, a microarray, or a hyperspectral imaging system (see, e.g., Vo-Dinh et al., IEEE Eng. Med. Biol. Mag. 23:40-49 (2004)). In one embodiment, a computer terminal may be connected to the detection module. For instance, the detection module may detect a label that selectively binds to cells of interest.

In another example, a capture module is fluidically coupled to a separation or enrichment device of the invention. For example, a capture module includes one or more binding moieties that selectively bind a particular cell type, e.g., a cancer cell or other rare cell. In capture module embodiments that include an array of obstacles, the obstacles may include such binding moieties.

Additionally, a cell counting module, e.g., a Coulter counter, may be fluidically coupled to a separation or enrichment device of the invention. Other modules, e.g., a programmable heating unit, may alternatively be fluidically coupled.

The methods of the invention may be employed in connection with any enrichment or analytical device, either on the same device or in different devices. Examples include affinity columns, particle sorters, e.g., fluorescent activated cell sorters, capillary electrophoresis, microscopes, spectrophotometers, sample storage devices, and sample preparation devices. Microfluidic devices are of particular interest in connection with the systems described herein.

Exemplary analytical devices include devices useful for size, shape, or deformability based enrichment of particles, including filters, sieves, and enrichment or separation devices, e.g., those described in International Publication Nos. 2004/029221 and 2004/113877, Huang et al. Science 304:987-990 (2004), U.S. Publication No. 2004/0144651, U.S. Pat. Nos. 5,837,115 and 6,692,952, and U.S. Application Nos. 60/703,833, 60/704,067, and 11/227,904; devices useful for affinity capture, e.g., those described in International Publication No. 2004/029221 and U.S. application Ser. No. 11/071,679; devices useful for preferential lysis of cells in a sample, e.g., those described in International Publication No. 2004/029221, U.S. Pat. No. 5,641,628, and U.S. Application No. 60/668,415; devices useful for arraying cells, e.g., those described in International Publication No. 2004/029221, U.S. Pat. No. 6,692,952, and U.S. application Ser. Nos. 10/778,831 and 11/146,581; and devices useful for fluid delivery, e.g., those described in U.S. application Ser. Nos. 11/071,270 and 11/227,469. Two or more devices may be combined in series, e.g., as described in International Publication No. 2004/029221.

Methods of Fabrication

Devices of the invention may be fabricated using techniques well known in the art. The choice of fabrication technique will depend on the material used for the device and the size of the array. Exemplary materials for fabricating the devices of the invention include glass, silicon, steel, nickel, polymers, e.g., poly(methylmethacrylate) (PMMA), polycarbonate, polystyrene, polyethylene, polyolefins, silicones (e.g., poly(dimethylsiloxane)), polypropylene, cis-polyisoprene (rubber), poly(vinyl chloride) (PVC), poly(vinyl acetate) (PVAc), polychloroprene (neoprene), polytetrafluoroethylene (Teflon), poly(vinylidene chloride) (SaranA), and cyclic olefin polymer (COP) and cyclic olefin copolymer (COC), and combinations thereof. Other materials are known in the art. For example, deep Reactive Ion Etch (DRIE) is used to fabricate silicon-based devices with small gaps, small obstacles and large aspect ratios (ratio of obstacle height to lateral dimension). Thermoforming (embossing, injection molding) of plastic devices may also be used, e.g., when the smallest lateral feature is >20 microns and the aspect ratio of these features is ≦10. Additional methods include photolithography (e.g., stereolithography or x-ray photolithography), molding, embossing, silicon micromachining, wet or dry chemical etching, milling, diamond cutting, Lithographie Galvanoformung and Abformung (LIGA), and electroplating. For example, for glass, traditional silicon fabrication techniques of photolithography followed by wet (KOH) or dry etching (reactive ion etching with fluorine or other reactive gas) may be employed. Techniques such as laser micromachining may be adopted for plastic materials with high photon absorption efficiency. This technique is suitable for lower throughput fabrication because of the serial nature of the process. For mass-produced plastic devices, thermoplastic injection molding, and compression molding may be suitable. Conventional thermoplastic injection molding used for mass-fabrication of compact discs (which preserves fidelity of features in sub-microns) may also be employed to fabricate the devices of the invention. For example, the device features are replicated on a glass master by conventional photolithography. The glass master is electroformed to yield a tough, thermal shock resistant, thermally conductive, hard mold. This mold serves as the master template for injection molding or compression molding the features into a plastic device. Depending on the plastic material used to fabricate the devices and the requirements on optical quality and throughput of the finished product, compression molding or injection molding may be chosen as the method of manufacture. Compression molding (also called hot embossing or relief imprinting) has the advantages of being compatible with high molecular weight polymers, which are excellent for small structures and may replicate high aspect ratio structures but has longer cycle times. Injection molding works well for low aspect ratio structures and is most suitable for low molecular weight polymers.

A device may be fabricated in one or more pieces that are then assembled. Layers of a device may be bonded together by clamps, adhesives, heat, anodic bonding, or reactions between surface groups (e.g., wafer bonding). Alternatively, a device with channels in more than one plane may be fabricated as a single piece, e.g., using stereolithography or other three-dimensional fabrication techniques.

To reduce non-specific adsorption of cells or compounds released by lysed cells onto the channel walls, one or more channel walls may be chemically modified to be non-adherent or repulsive. The walls may be coated with a thin film coating (e.g., a monolayer) of commercial non-stick reagents, such as those used to form hydrogels. Additional examples chemical species that may be used to modify the channel walls include oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers may also be employed to repel oppositely charged species. The type of chemical species used for repulsion and the method of attachment to the channel walls will depend on the nature of the species being repelled and the nature of the walls and the species being attached. Such surface modification techniques are well known in the art. The walls may be functionalized before or after the device is assembled. The channel walls may also be coated in order to capture materials in the sample, e.g., membrane fragments or proteins.

Methods of Operation

Devices of the invention may be employed in any application where the production of a sample enriched in particles above or below a critical size is desired. A preferred use of the device is to produce samples enriched in CTCs or other rare cells. Once an enriched sample is produced, it may be collected for analysis or otherwise manipulated.

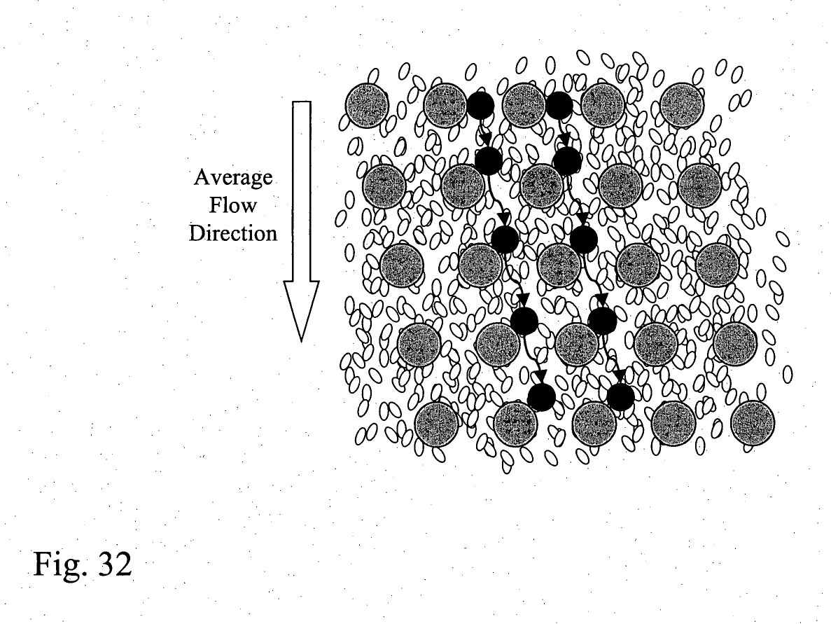

Devices of the invention may be employed in concentrated samples, e.g., where particles are touching, hydrodynamically interacting with each other, or exerting an effect on the flow distribution around another particle. For example, the method may enrich CTCs from other cells in whole blood from a human donor. Human blood typically contains ˜45% of cells by volume. Cells are in physical contact and/or coupled to each other hydrodynamically when they flow through the array. FIG. 32 shows schematically that cells are densely packed inside an array and could physically interact with each other.

Enrichment

In one embodiment, devices of the invention are employed to produce a sample enriched in particles of a desired hydrodynamic size. Applications of such enrichment include concentrating CTCs or other cells of interest, and size fractionization, e.g., size filtering (selecting cells in a particular size range). Devices may also be used to enrich components of cells, e.g., nuclei. Desirably, the methods of the invention retain at least 50%, 75%, 80%, 81%, 82%, 83%, 84%, 85%, 86%, 87%, 88%, 89%, 90%, 91%, 92%, 93%, 94%, 95%, 96%, 97%, 98%, or even 99% of the desired particles compared to the initial mixture, while potentially enriching the desired particles by a factor of at least 100, 1,000, 10,000, 100,000, 1,000,000, 10,000,000, or even 100,000,000 relative to one or more non-desired particles. Desirably, if a device produces any output sample in addition to the enriched sample, this additional output sample contains less than 50%, 25%, 20%, 19%, 18%, 17%, 16%, 15%, 14%, 13%, 12%, 11%, 10%, 9%, 8%, 7%, 6%, 5%, 4%, 3%, 2%, or even 1% of the desired particles compared to the initial mixture. The enrichment may also result in a dilution of the enriched particles compared to the original sample, although the concentration of the enriched particles relative to other particles in the sample has increased. Preferably, the dilution is at most 90%, e.g., at most 75%, 50%, 33%, 25%, 10%, or 1%.

In a preferred embodiment, the device produces a sample enriched in a rare particles, e.g., cells. In general, a rare particle is a particle that is present as less than 10% of a sample. Rare particles include, depending on the sample, rare cells, e.g., CTCs, epithelial cells, fetal cells, stem cells (e.g., undifferentiated), bone marrow cells, progenitor cells, foam cells, mesenchymal cells, endothelial cells, endometrial cells, trophoblasts, cancer cells, immune system cells (host or graft), connective tissue cells, bacteria, fungi, and pathogens (e.g., bacterial or protozoa). Rare particles also include viruses, as well as cellular components such as organelles (e.g., mitochondria and nuclei). Rare particles may be isolated from samples including bodily fluids, e.g., blood, or environmental sources, e.g., pathogens in water samples. Fetal red blood cells may be enriched from maternal peripheral blood, e.g., for the purpose of determining sex and identifying aneuploidies or genetic characteristics, e.g., mutations, in the developing fetus. CTCs, which are of epithelial type and origin, may also be enriched from peripheral blood for the purpose of diagnosis and monitoring therapeutic progress. Circulating endothelial cells may be similarly enriched from peripheral blood. Bodily fluids or environmental samples may also be screened for pathogens, e.g., for coliform bacteria, blood borne illnesses such as sepsis, or bacterial or viral meningitis. Rare cells also include cells from one organism present in another organism, e.g., an in cells from a transplanted organ.

In addition to enrichment of rare particles, devices of the invention may be employed for preparative applications. An exemplary preparative application includes generation of cell packs from blood. Devices of the invention may be configured to produce fractions enriched in platelets, red blood cells, and white cells. By using multiplexed devices or multistage devices, all three cellular fractions may be produced in parallel or in series from the same sample. In other embodiments, the device may be employed to separate nucleated from non-nucleated cells, e.g., from cord blood sources.

Using the devices of the invention is advantageous in situations where the particles being enriched are subject to damage or other degradation. As described herein, devices of the invention may be designed to enrich cells with a minimum number of collisions between the cells and obstacles. This minimization reduces mechanical damage to cells and also prevents intracellular activation of cells caused by the collisions. This gentle handling of the cells preserves the limited number of rare cells in a sample, prevents rupture of cells leading to contamination or degradation by intracellular components, and prevents maturation or activation of cells, e.g., stem cells or platelets. In preferred embodiments, cells are enriched such that fewer than 30%, 10%, 5%, 1%, 0.1%, or even 0.01% are activated or mechanically lysed.

FIG. 33A shows a typical size distribution of cells in human peripheral blood. The white blood cells range from ˜4 μm to ˜12 μm, whereas the red blood cells are ˜1.5-3 μm (short axis). FIG. 33B shows that CTCs are generally significantly larger than blood cells, with the majority of CTCs ranging from ˜8 to ˜22 μm. Thus, a size-based enrichment using a device of the invention, in which the size cutoff is chosen to be, e.g., 12 μm (FIG. 33C), would be effective in enriching CTCs from other blood cells. Any cell population with a similar distribution to CTCs may be similarly enriched from blood cells (FIG. 33D).

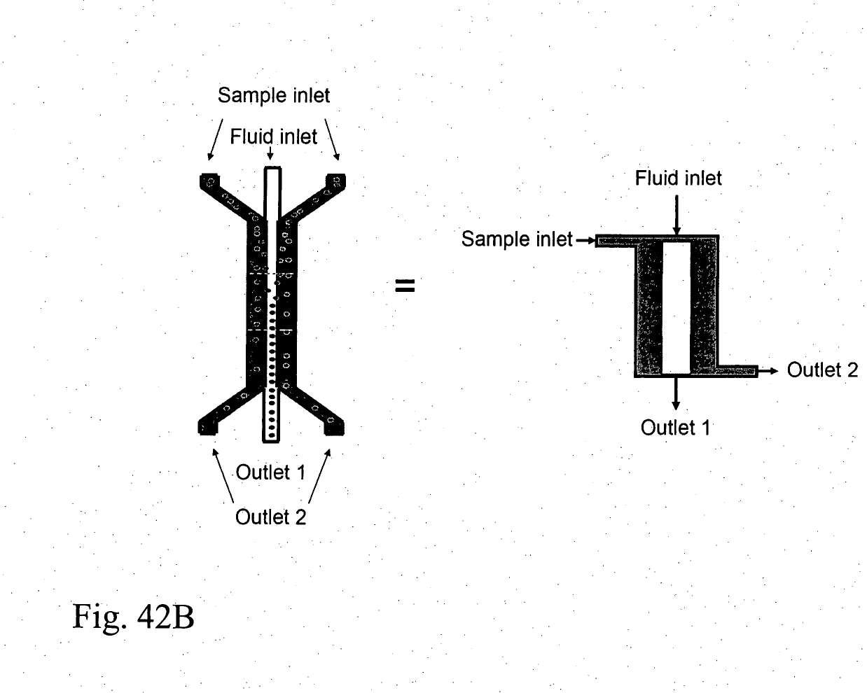

In an alternative embodiment, a cellular sample is added through a sample inlet of the device, and buffer medium is added through the fluid inlet (FIG. 42A). Cells below the critical size move through the device undeflected, emerging from the edge outlets in their original sample medium. Cells above the critical size, e.g., epithelial cells, in particular, CTCs, are deflected and emerge from the center outlet contained in the buffer medium added through the fluid inlet. Operation of the device thus produces samples enriched in cells above and below the critical size. Because epithelial cells are among the largest cells in the bloodstream, the size and geometry of the gaps of the device may be chosen so as to direct virtually all other cell types to the edge outlets, while producing a sample from the center outlet that is substantially enriched in epithelial cells after a single pass through the device.

A device of the invention need not be duplexed as shown in FIG. 42A in order to operate as described herein. The schematized representation shown in FIG. 42B may represent either a duplexed device or a single array.

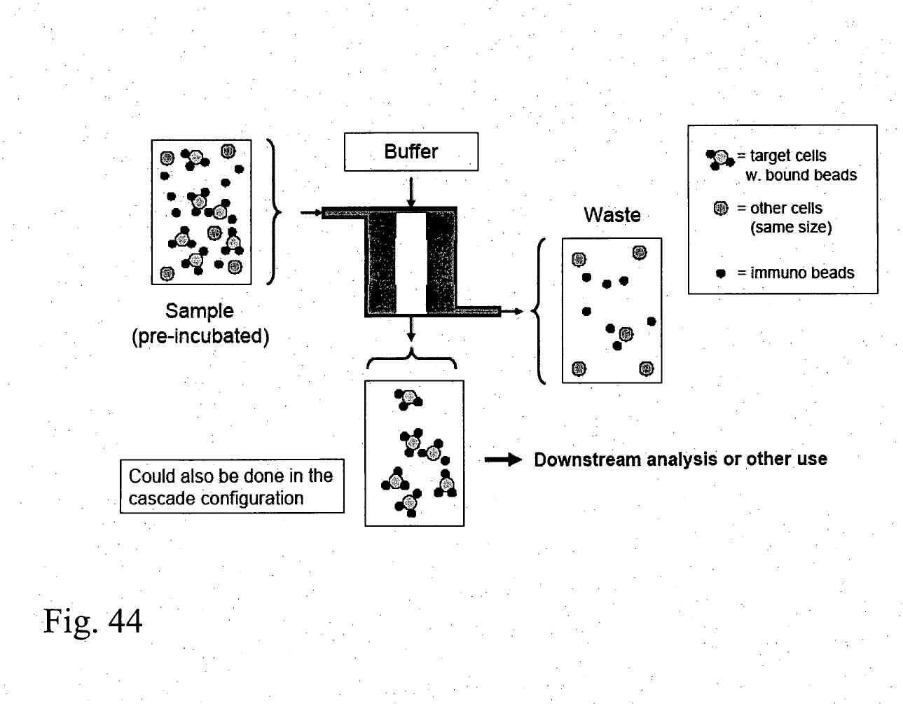

Enrichment may be enhanced in numerous ways. For example, target cells may be labeled with immunoaffinity beads, thereby increasing their size (as depicted in FIG. 44). In the case of epithelial cells, e.g., CTCs, this may further increase their size and thus result in an even more efficient enrichment. Alternatively, the size of smaller cells may be increased to the extent that they become the largest objects in solution or occupy a unique size range in comparison to the other components of the cellular sample, or so that they copurify with other cells. The hydrodynamic size of a labeled target cell may be at least 10%, 100%, or even 1,000% greater than the hydrodynamic size of such a cell in the absence of label. Beads may be made of polystyrene, magnetic material, or any other material that may be adhered to cells. Desirably, such beads are neutrally buoyant so as not to disrupt the flow of labeled cells through the device of the invention.

Enrichment methods of the invention include devices that include obstacles that are capable of selectively capturing cells of interest, e.g., epithelial cells, e.g., CTCs.

The methods of the invention may also be used to deplete or remove an analyte from a cellular sample, for example, by producing a sample enriched in another analyte using the above-described methods. For example, a cellular sample may be depleted of cells having a hydrodynamic size less than or equal to 12 microns by enriching for cells having a hydrodynamic size greater than 12 microns. Any method of depletion or removal may be used in conjunction with the arrays and devices of the invention. In methods of the invention featuring depletion of removal of an analyte, sample processing may be continuous and may occur in vivo or ex vivo. Furthermore, in some embodiments, if the analyte to be depleted or removed is retained in a device of the invention, the analyte may be released from the device by applying a hypertonic solution to said device. The analyte may then be detected in the effluent from the device.

Alteration

In other embodiments, in addition to enrichment, CTCs or other cells of interest are contacted with an altering reagent that may chemically or physically alter the particle or the fluid in the suspension. Such applications include purification, buffer exchange, labeling (e.g., immunohistochemical, magnetic, and histochemical labeling, cell staining, and flow in-situ fluorescence hybridization (FISH)), cell fixation, cell stabilization, cell lysis, and cell activation.

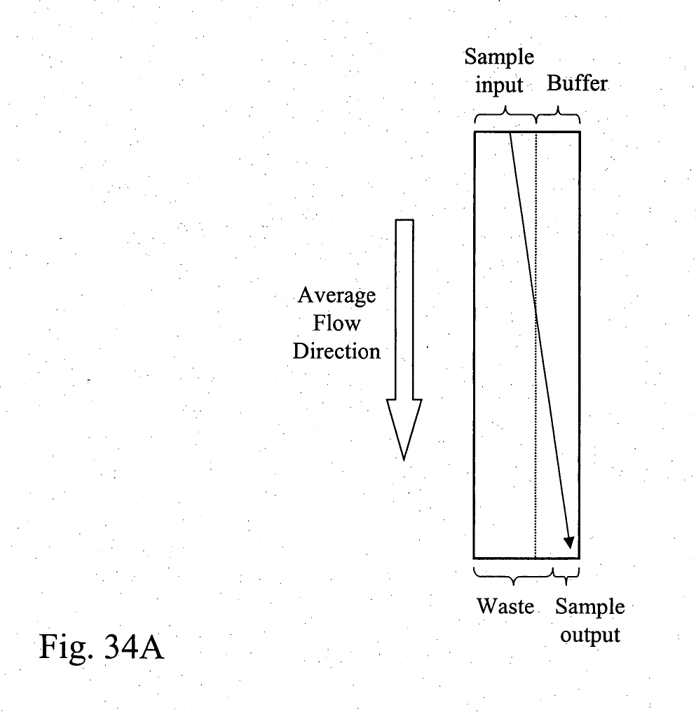

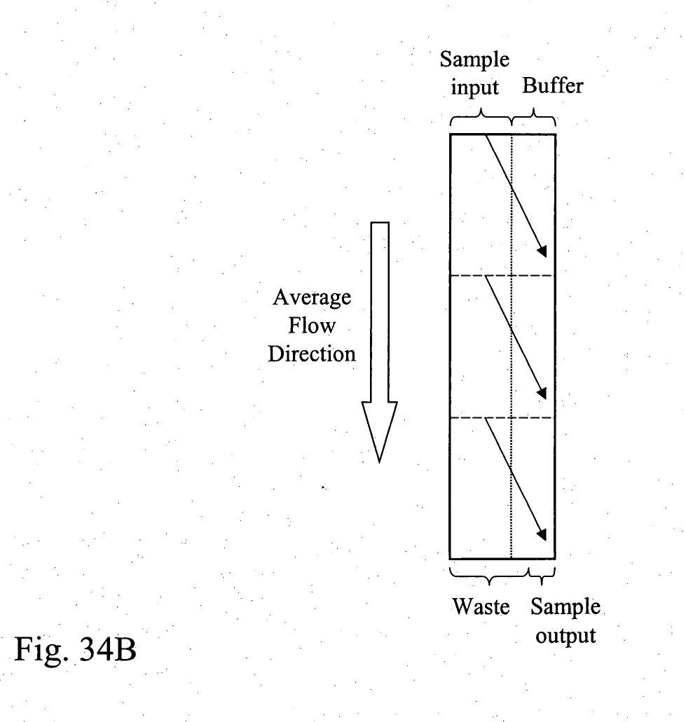

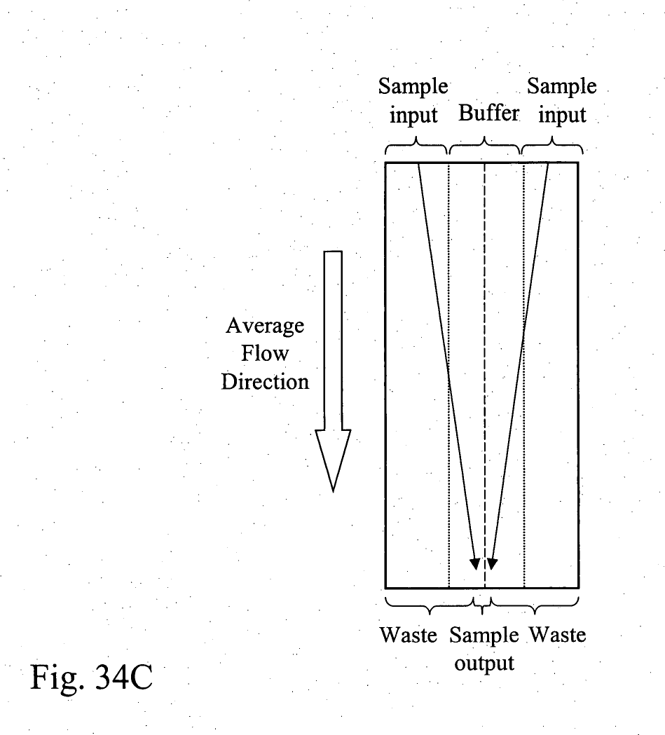

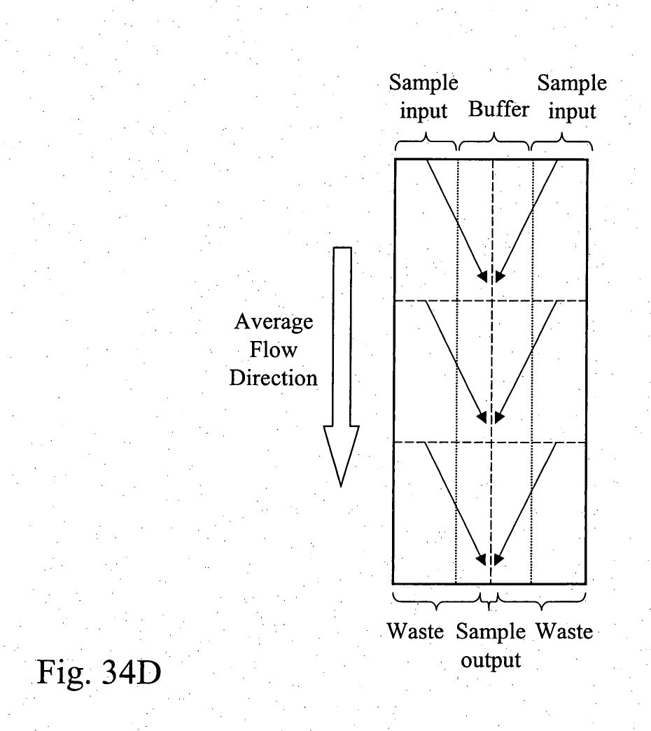

Such methods allow for the transfer of particles, e.g., CTCs, from a sample into a different liquid. FIG. 34A shows this effect schematically for a single stage device, FIG. 34B shows this effect for a multistage device, FIG. 34C shows this effect for a duplex array, and FIG. 34D shows this effect for a multistage duplex array. By using such methods, blood cells may be separated from plasma. Such transfers of particles from one liquid to another may be also employed to effect a series of alterations, e.g., Wright staining blood on-chip. Such a series may include reacting a particle with a first reagent and then transferring the particle to a wash buffer, and then another reagent.

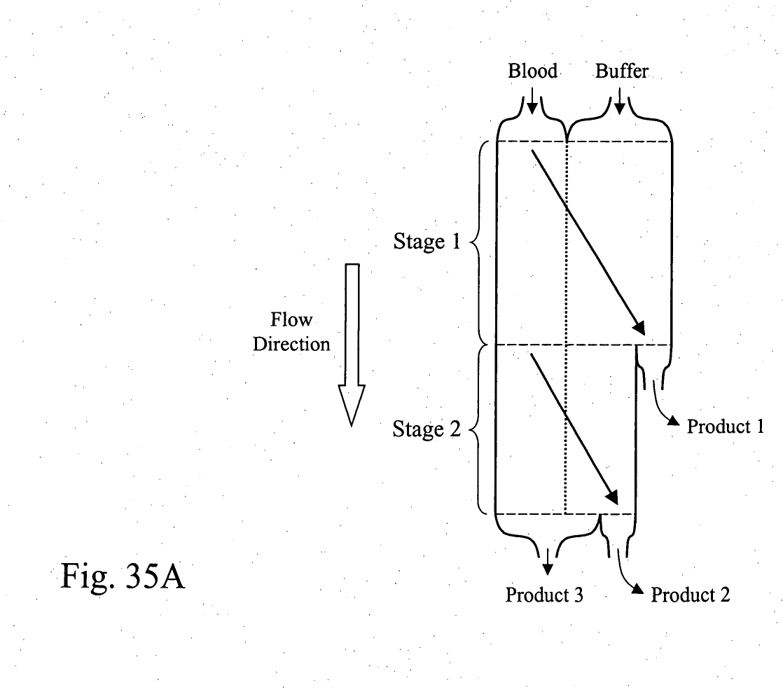

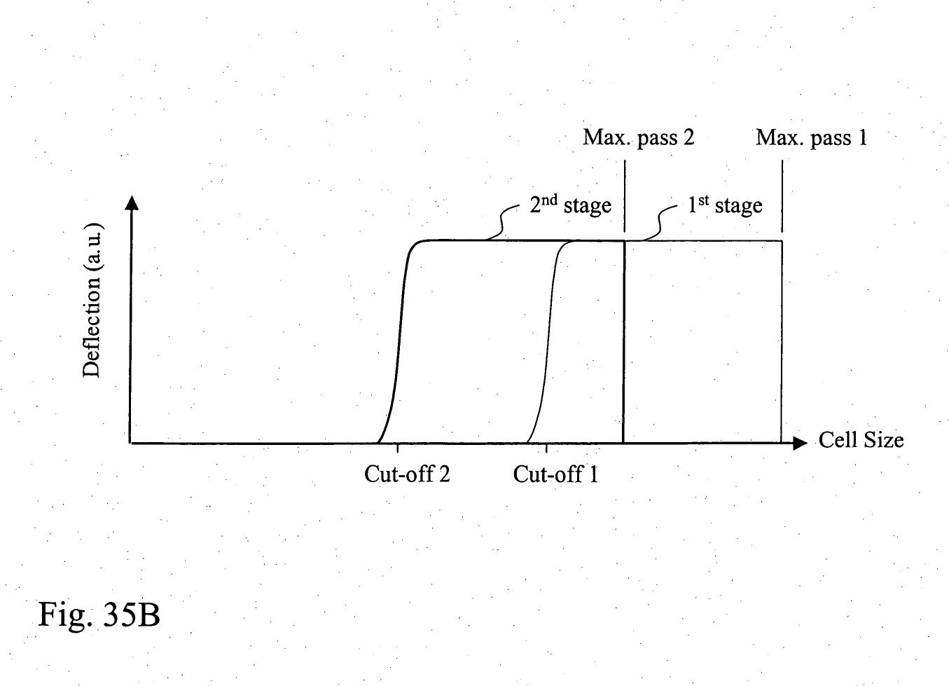

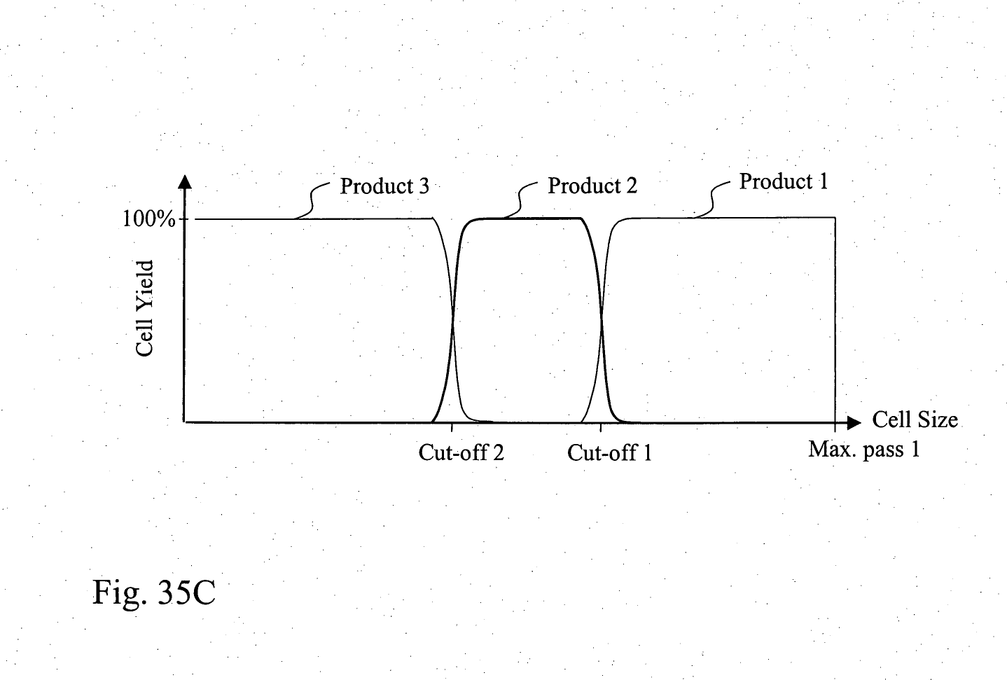

FIGS. 35A-35C illustrate a further example of alteration in a two stage device having two bypass channels. In this example, large blood particles are moved from blood to buffer and collected in stage 1, medium blood particles are moved from blood to buffer in stage 2, and small cells that are not moved from the blood in stage are collected also collected. FIG. 35B illustrates the size cut-off of the two stages, and FIG. 35C illustrates the size distribution of the three fractions collected.

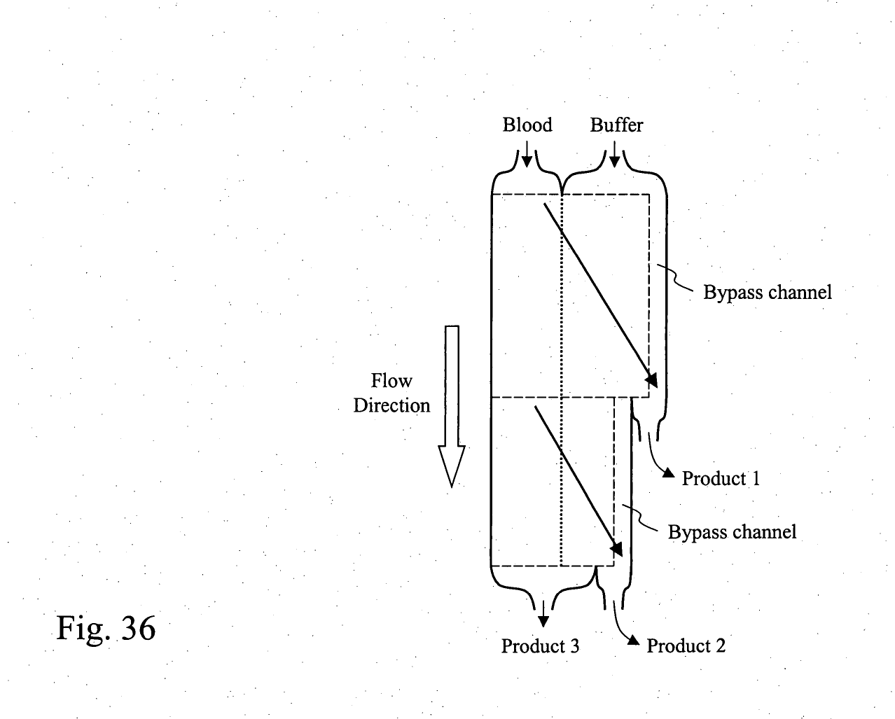

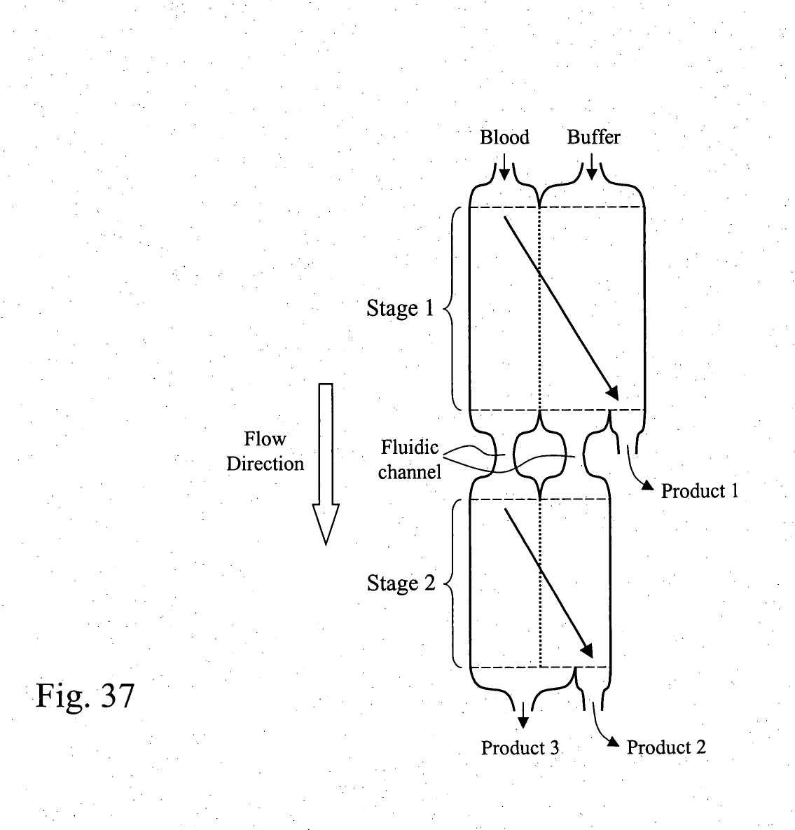

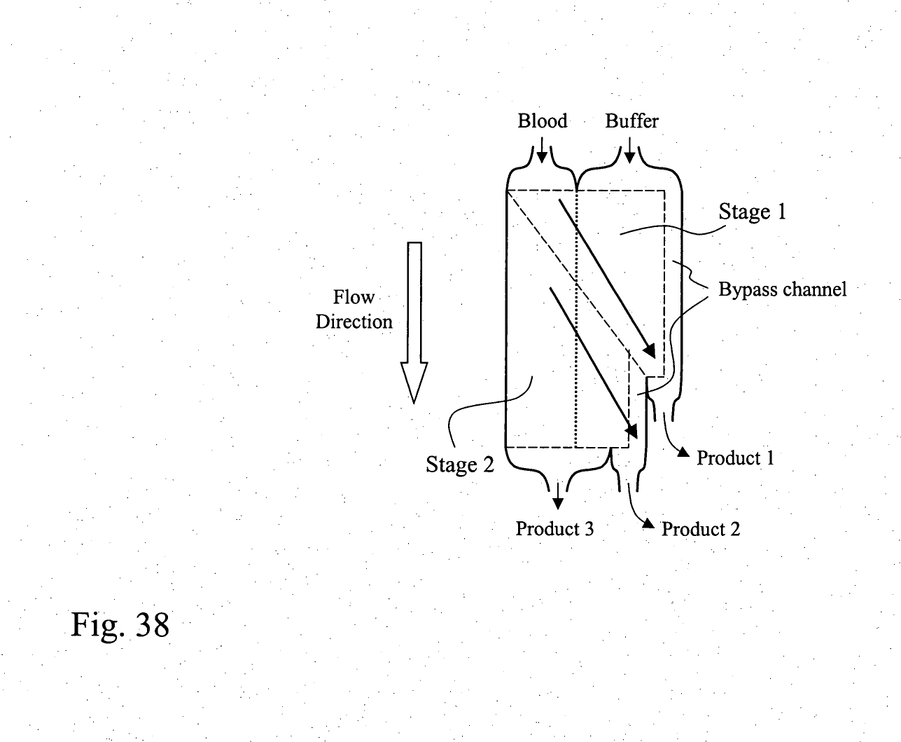

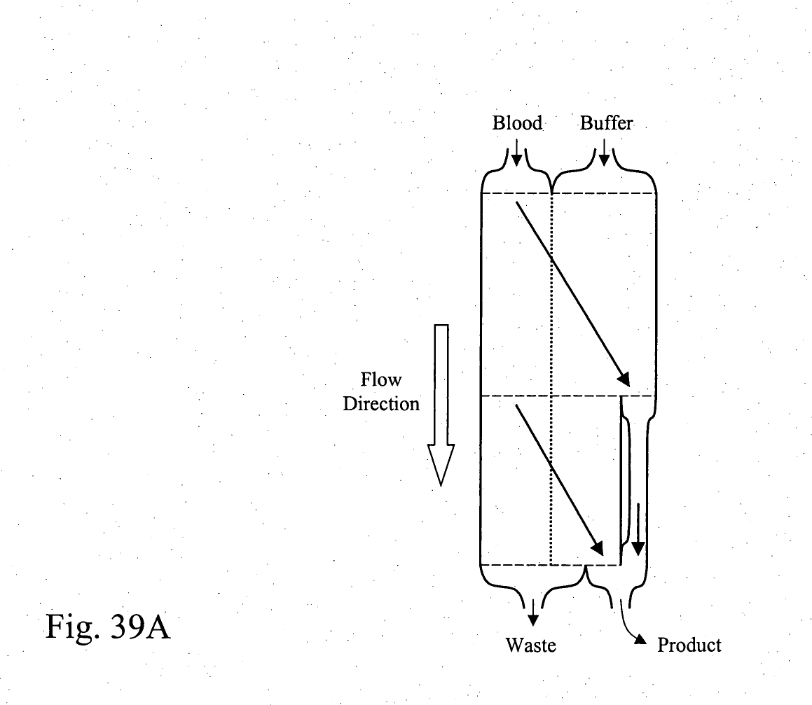

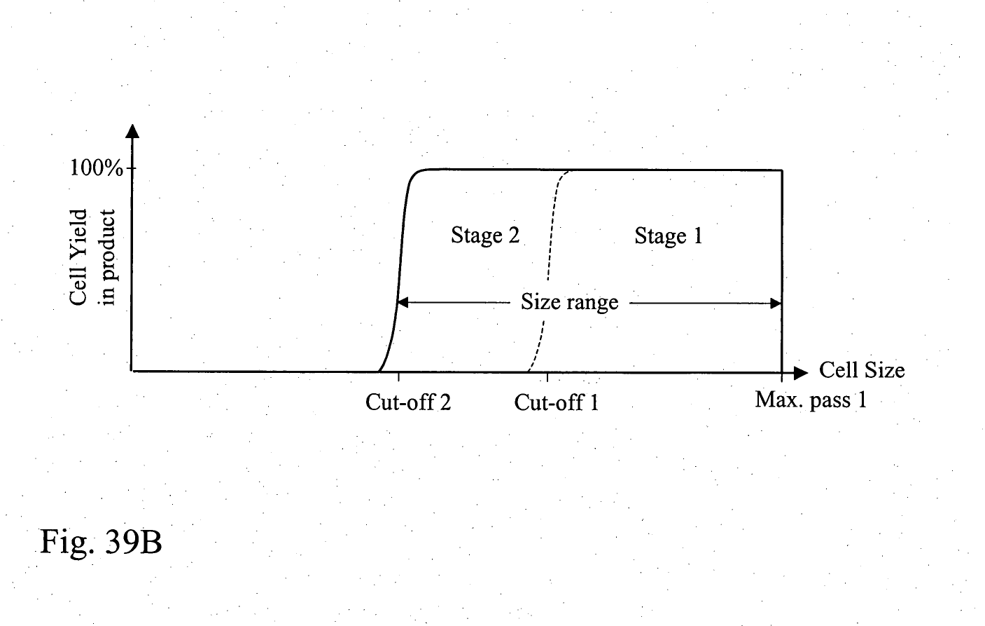

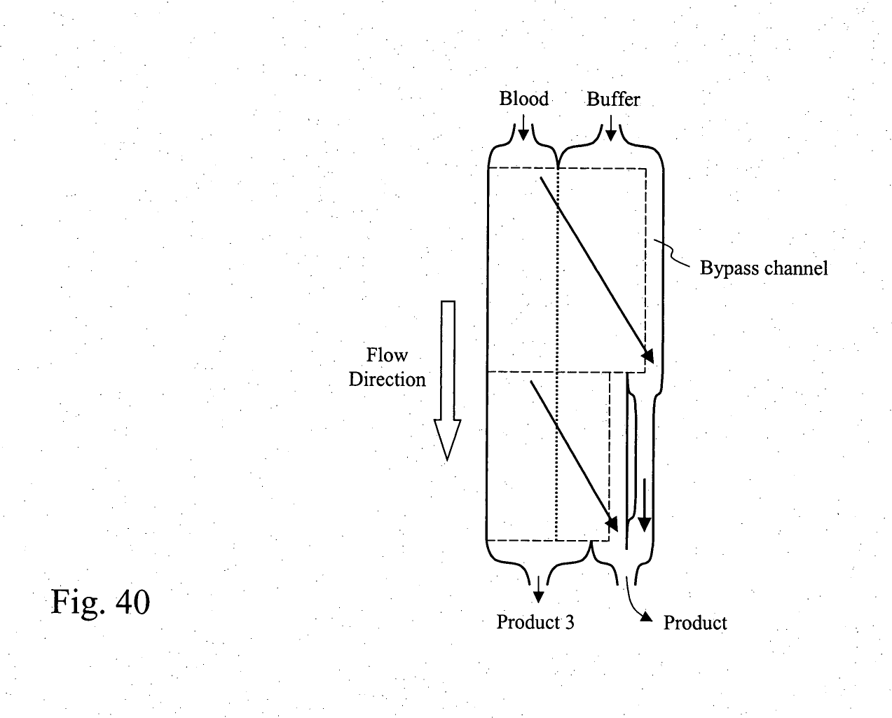

FIG. 36 illustrates an example of alteration in a two stage device having bypass channels that are disposed between the lateral edge of the array and the channel wall. FIG. 37 illustrates a device similar to that in FIG. 36, except that the two stages are connected by fluidic channels. FIG. 38 illustrates alteration in a device having two stages with a small footprint. FIGS. 39A-39B illustrate alteration in a device in which the output from the first and second stages is captured in a single channel. FIG. 40 illustrates another device for use in the methods of the invention.

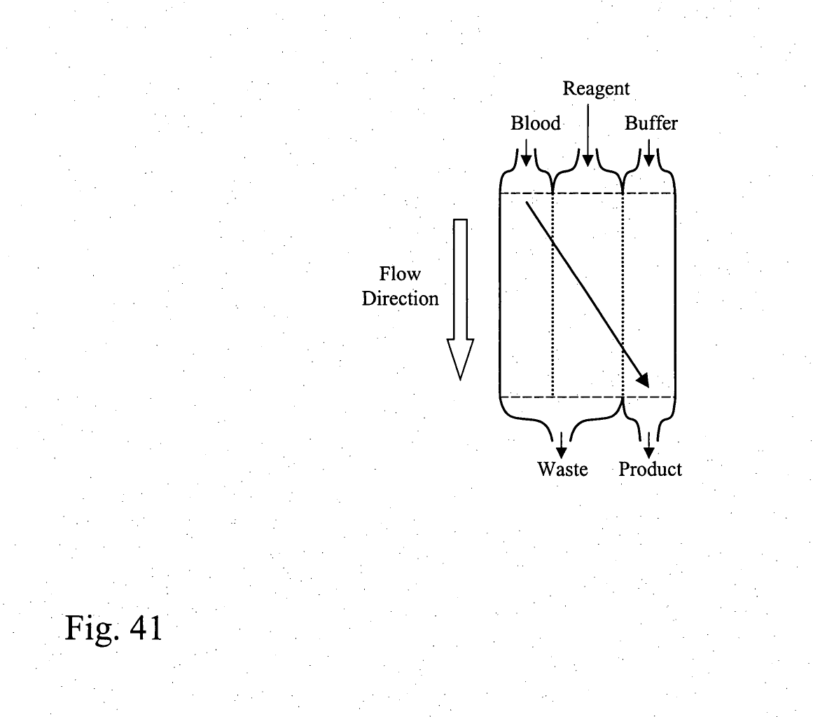

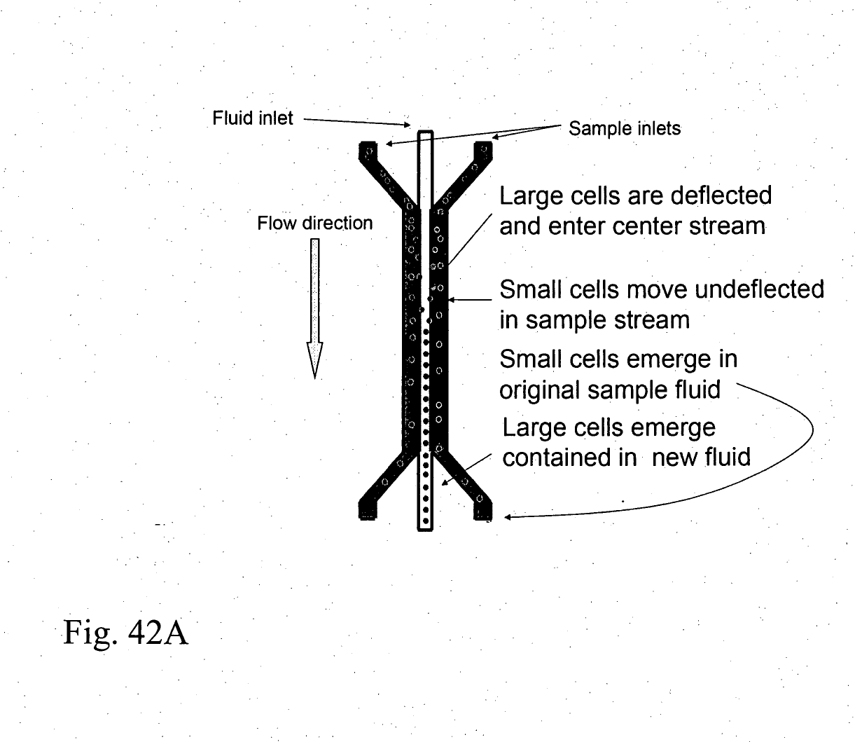

FIG. 41 illustrates the use of a device to perform multiple, sequential alterations on a particle. In this device a blood particles is moved from blood into a regent that reacts with the particle, and the reacted particle is then moved into a buffer, thereby removing the unreacted reagent or reaction byproducts. Additional steps may be added.

Enrichment and alteration may also be combined, e.g., where desired cells are contacted with a lysing reagent and cellular components, e.g., nuclei, are enriched based on size. In another example, particles may be contacted with particulate labels, e.g., magnetic beads, which bind to the particles. Unbound particulate labels may be removed based on size.

Separation of Free Labeling Reagent from Labeling Reagent Bound to Cells

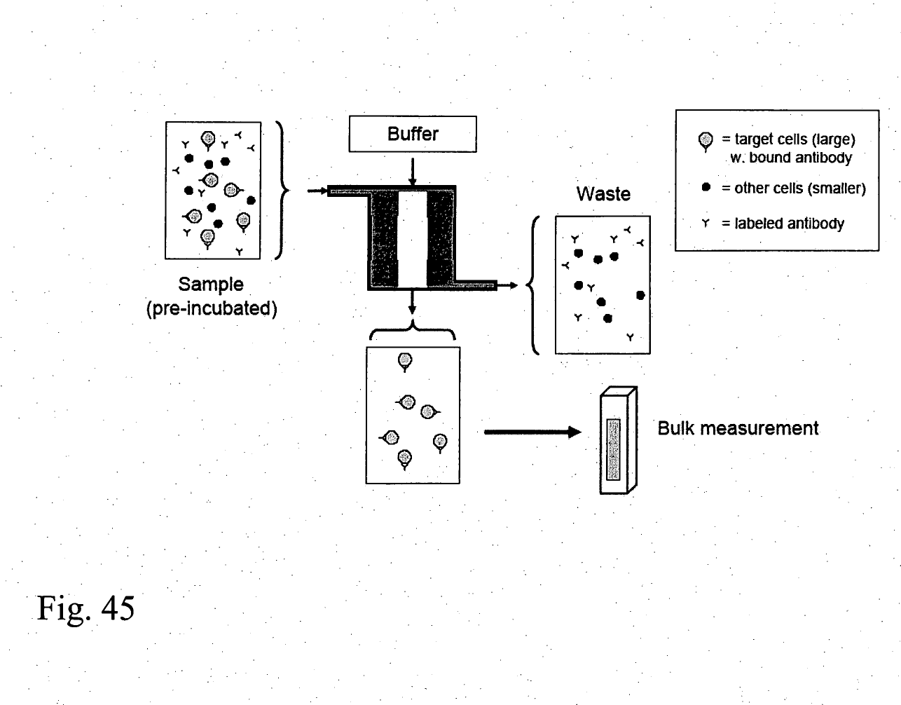

Devices of the invention may be employed in order to separate free labeling reagent from labeling reagent bound to CTCs or other cells. As shown in FIG. 45, a labeling reagent may be pre-incubated with a cellular sample prior to introduction to the device. Desirably, the labeling reagent specifically or preferentially binds the cell population of interest, e.g., epithelial cells such as CTCs. Exemplary labeling reagents include antibodies, quantum dots, phage, aptamers, fluorophore-containing molecules, enzymes capable of carrying out a detectable chemical reaction, or functionalized beads. Generally, the labeling reagent is smaller than the cell of interest, or the cell of interest bound to the bead; thus, when the cellular sample combined with the labeling reagent is introduced to the device, free labeling reagent moves through the device undeflected and emerges from the edge outlets, while bound labeling reagent emerges from the center outlet along with epithelial cells. Advantageously, this method simultaneously achieves size separation and separation of free labeling reagent from bound labeling reagent. Additionally, this method of separation facilitates downstream sample analysis without the need for a release step or destructive methods of analysis, as described below.

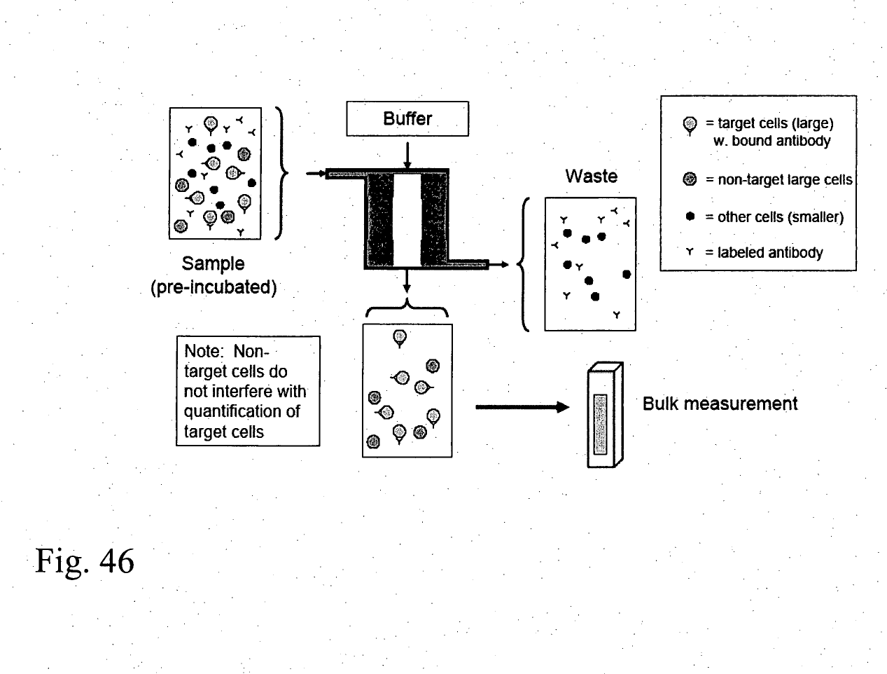

FIG. 46 shows a more general case, in which the enriched labeled sample contains a population of non-target cells that co-separate with the target cells due to similar size. The non-target cells do not interfere with downstream sample analysis that relies on detection of the bound labeling reagent, because this reagent binds selectively to the cells of interest.

Buffer Exchange

Devices of the invention may be employed for purposes of buffer exchange. To achieve this result, a protocol similar to that used for enrichment is followed: a cellular sample is added through a sample inlet of the device, and the desired final buffer medium is added through a fluid inlet. As described above, cells above the critical size are deflected and enter the buffer.

Concentration

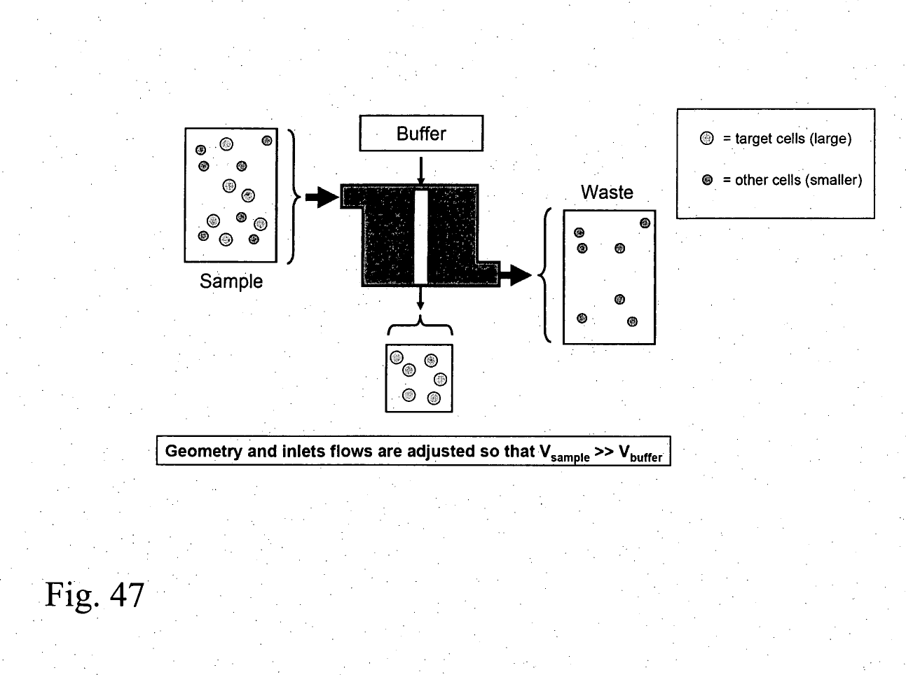

Devices of the invention may be employed in order to concentrate a cellular sample of interest, e.g., a sample containing CTCs. As shown in FIG. 47, a cellular sample is introduced to the sample inlet of the device. By reducing the volume of buffer introduced into the fluid inlet so that this volume is significantly smaller than the volume of the cellular sample, concentration of target cells in a smaller volume results. This concentration step may improve the results of any downstream analysis performed.

Cell Lysis

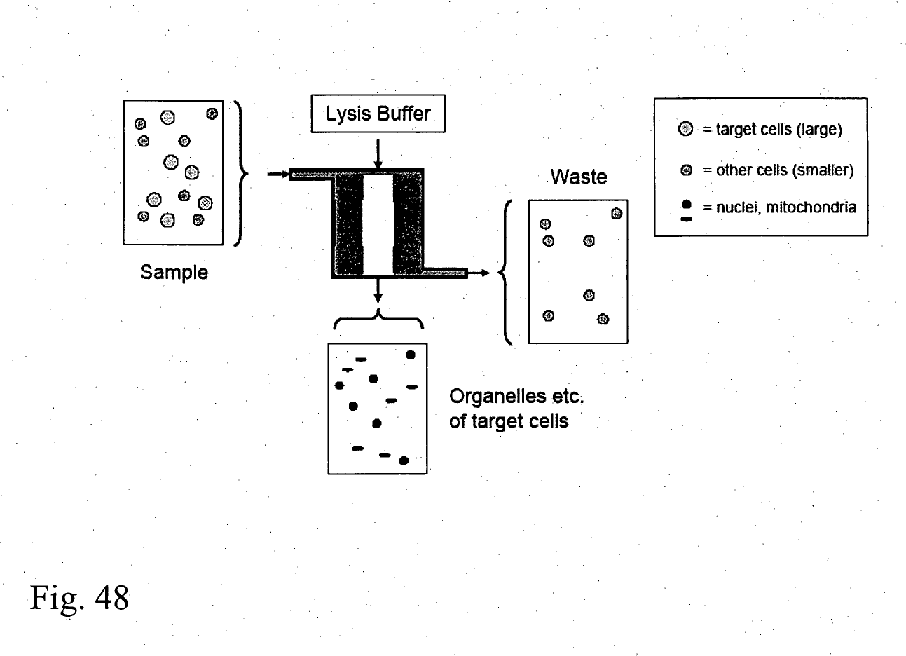

Devices of the invention may be employed for purposes of cell lysis. To achieve this, a protocol similar to that used for enrichment is followed: a cellular sample is added through a sample inlet of the device (FIG. 48), and lysis buffer is added through the fluid inlet. As described above, cells above the critical size are deflected and enter the lysis buffer, leading to lysis of these cells. As a result, the sample emerging from the center outlet includes lysed cell components including organelles, while undeflected whole cells emerge from the other outlet. Thus, the device provides a method for selectively lysing target cells.

Multiple Stages

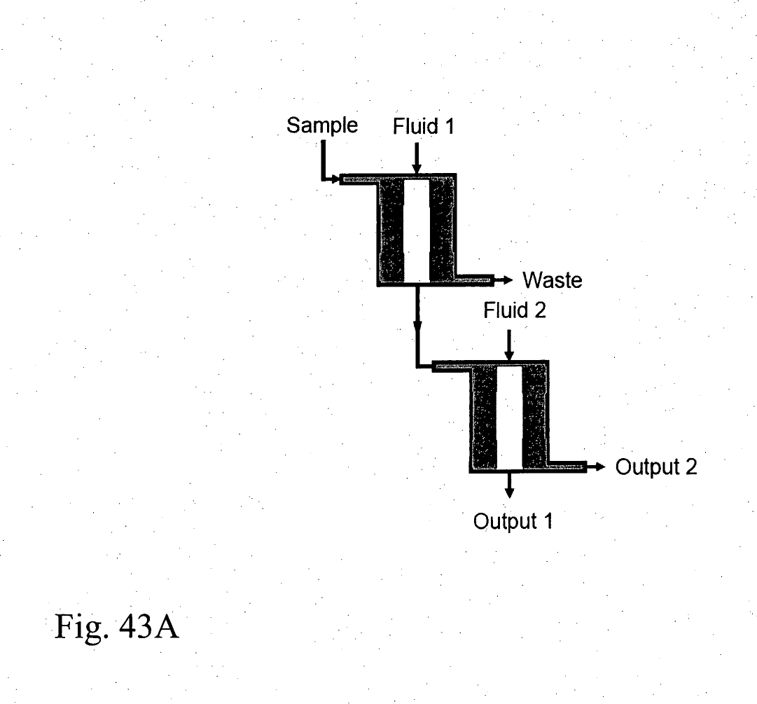

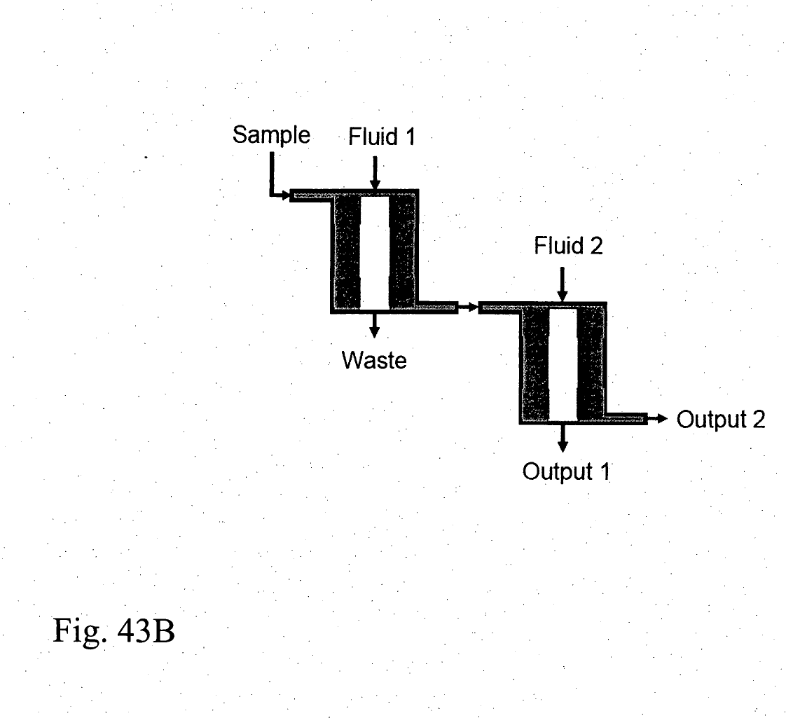

Devices of the invention may be joined together to provide multiple stages of enrichment and reaction. For example, FIG. 43A shows the “cascade” configuration, in which outlet 1 of one device is joined to a sample inlet of a second device. This allows for an initial enrichment step using the first device so that the sample introduced to the second device is already enriched for cells of interest. The two devices may have either identical or different critical sizes, depending on the intended application.

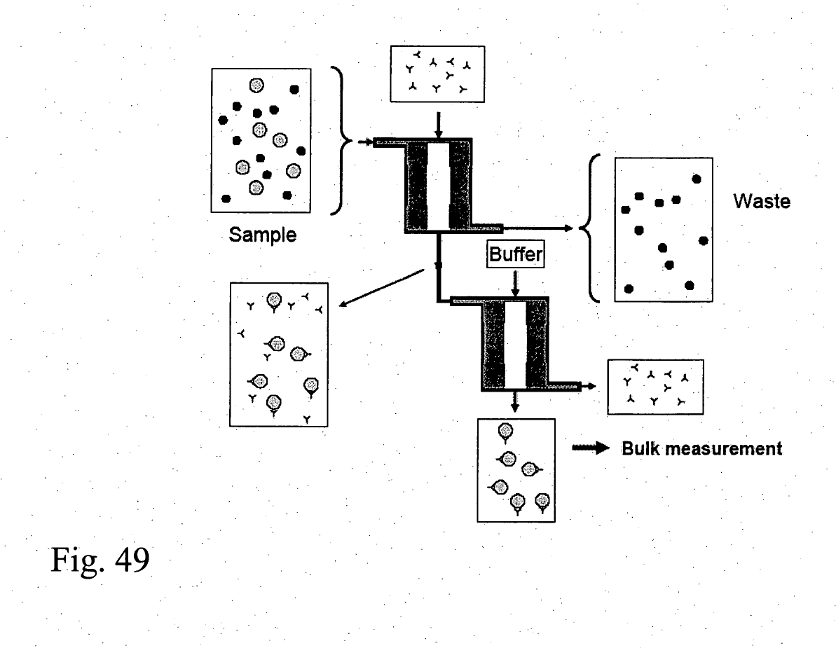

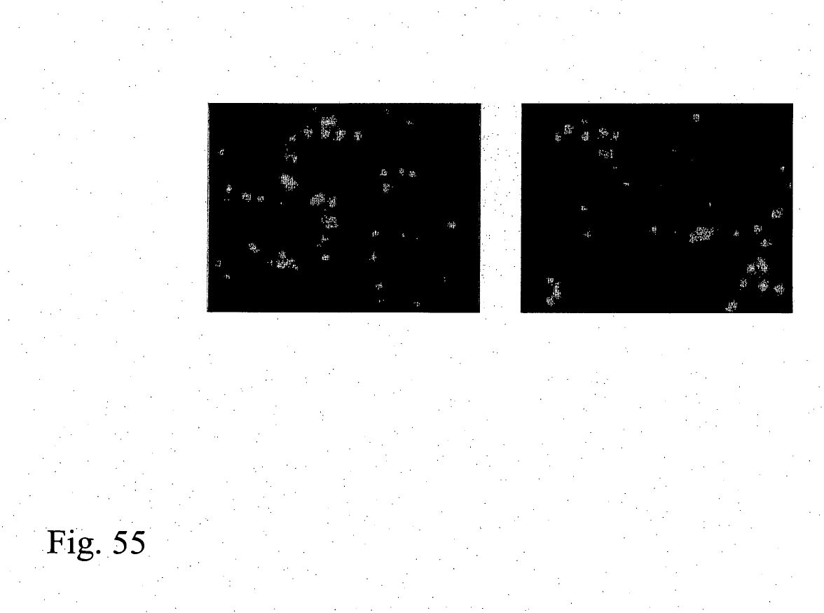

In FIG. 49, an unlabeled cellular sample is introduced to the first device in the cascade via a sample inlet, and a buffer containing labeling reagent is introduced to the first device via the fluid inlet. Epithelial cells, e.g., CTCs, are deflected and emerge from the center outlet in the buffer containing labeling reagent. This enriched labeled sample is then introduced to the second device in the cascade via a sample inlet, while buffer is added to the second device via the fluid inlet. Further enrichment of target cells and separation of free labeling reagent is achieved, and the enriched sample may be further analyzed. Alternatively, labeling reagent may be added directly to the sample emerging from the center outlet of the first device before introduction to the second device. The use of a cascade configuration may allow for the use of a smaller quantity or a higher concentration of labeling reagent at less expense than the single-device configuration of FIG. 55; in addition, any nonspecific binding that may occur is significantly reduced by the presence of an initial enrichment step using the first device.

An alternative configuration of two or more device stages is the “bandpass” configuration. FIG. 43B shows this configuration, in which outlet 2 of one device is joined to a sample inlet of a second device. This allows for an initial enrichment step using the first device so that the sample introduced to the second device contains cells that remained undeflected within the first device. This method may be useful when the cells of interest are not the largest cells in the sample; in this instance, the first stage may be used to reduce the number of large non-target cells by deflecting them to the center outlet. As in the cascade configuration, the two devices may have either identical or different critical sizes, depending on the intended application. For example, different critical sizes are appropriate for an application requiring the enrichment of epithelial cells, e.g., CTCs, in comparison with an application requiring the enrichment of smaller endothelial cells.

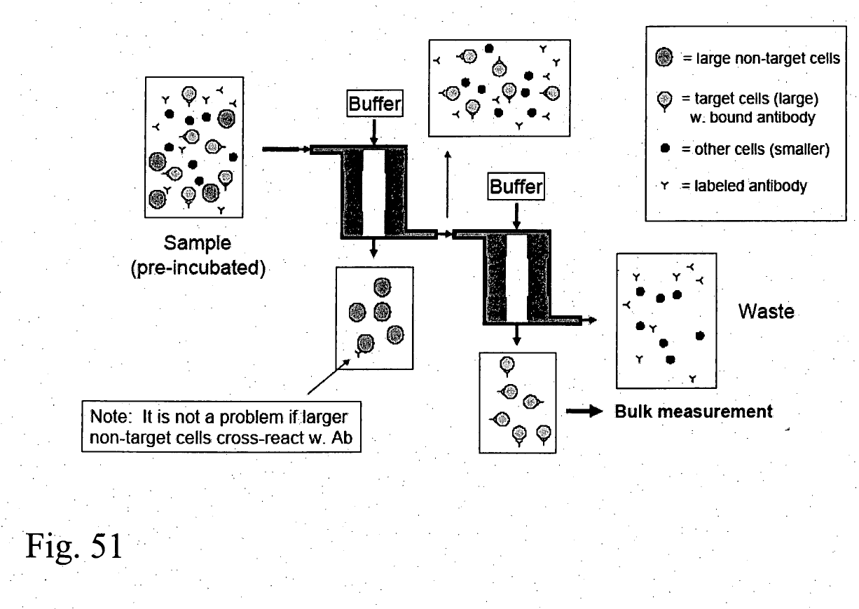

In FIG. 51, a cellular sample pre-incubated with labeling reagent is introduced to a sample inlet of the first device of the bandpass configuration, and a buffer is introduced to the first device via the fluid inlet. The first device is disposed in such a manner that large, non-target cells are deflected and emerge from the center outlet, while a mixture of target cells, small non-target cells, and labeling reagent emerge from outlet 2 of the first device. This mixture is then introduced to the second device via a sample inlet, while buffer is added to the second device via the fluid inlet. Enrichment of target cells and separation of free labeling reagent is achieved, and the enriched sample may be further analyzed. Non-specific binding of labeling reagent to the deflected cells in the first stage is acceptable in this method, as the deflected cells and any bound labeling reagent are removed from the system.

In any of the multiple device configurations described above, the devices and the connections joining them may be integrated into a single device. For example, a single cascade device including two or more stages is possible, as is a single bandpass device including two or more stages.

Downstream Analysis

A useful step for many diagnostic assays is the removal of free labeling reagent from the sample to be analyzed. As described above, devices of the invention are able to separate free labeling reagent from labeling reagent bound to cells, e.g., CTCs. It is then possible to perform a bulk measurement of the labeled sample without significant levels of background interference from free labeling reagent. For example, fluorescent antibodies selective for a particular epithelial cell marker such as EpCAM may be used. The fluorescent moiety may include Cy dyes, Alexa dyes, or other fluorophore-containing molecules. The resulting labeled sample is then analyzed by measuring the fluorescence of the resulting sample of labeled enriched cells using a fluorometer. Alternatively, a chromophore-containing label may be used in conjunction with a spectrometer, e.g., a UV or visible spectrometer. The measurements obtained may be used to quantify the number of target cells or all cells in the sample. Alternatively, the ratio of two cells types in the sample, e.g., the ratio of cancer cells to endothelial cells, may be determined. This ratio may be a ratio of the number of each type of cell, or alternatively it may be a ratio of any measured characteristic of each type of cell.

Any method of identifying cells, e.g., cells that have a cell surface marker associated with cancer, e.g., Ber-Ep4, CD34+, EpCAM, E-Cadherin, Mucin-1, Cytokeratin 8, EGFR, and leukocyte associated receptor (LAR), may be used. For example, an enriched sample of CTCs may be contacted with a device that includes a surface with one or more binding moieties that selectively bind one or more cells of the enriched sample. The binding moieties may include a polypeptide, e.g., an antibody or fragment thereof, e.g., monoclonal. For example, such a monoclonal antibody could be specific for EpCAM, e.g., anti-human EpCAM/TROP1 (catalog #AF960, R&D Systems).

Many other methods of measurement and labeling reagents are useful in the methods of the invention. Any imaging technique, e.g., hyperspectral imaging, may be used. Labeling antibodies, e.g., antibodies selective for any cancer marker, e.g., those listed in Table 1, may possess covalently bound enzymes that cleave a substrate, altering its absorbance at a given wavelength; the extent of cleavage is then quantified with a spectrometer. Colorimetric or luminescent readouts are possible, depending on the substrate used. Advantageously, the use of an enzyme label allows for significant amplification of the measured signal, lowering the threshold of detectability.

Quantum dots, e.g., Qdots® from QuantumDot Corp., may also be utilized as a labeling reagent that is covalently bound to a targeting antibody. Qdots are resistant to photobleaching and may be used in conjunction with two-photon excitation measurements.

Other possible labeling reagents useful in the methods of the invention are phage. Phage display is a technology in which binding peptides are displayed by engineered phage strains having strong binding affinities for a target protein, e.g., those found on the surface of cells of interest. The peptide sequence corresponding to a given phage is encoded in that phage's nucleic acid, e.g., DNA or RNA. Thus, phage are useful labeling reagents in that they are small relative to epithelial cells such as CTCs and thus may be easily separated, and they additionally carry nucleic acid that may be analyzed and quantified using PCR or similar techniques, enabling a quantitative determination of the number of cells present in an enriched bound sample.

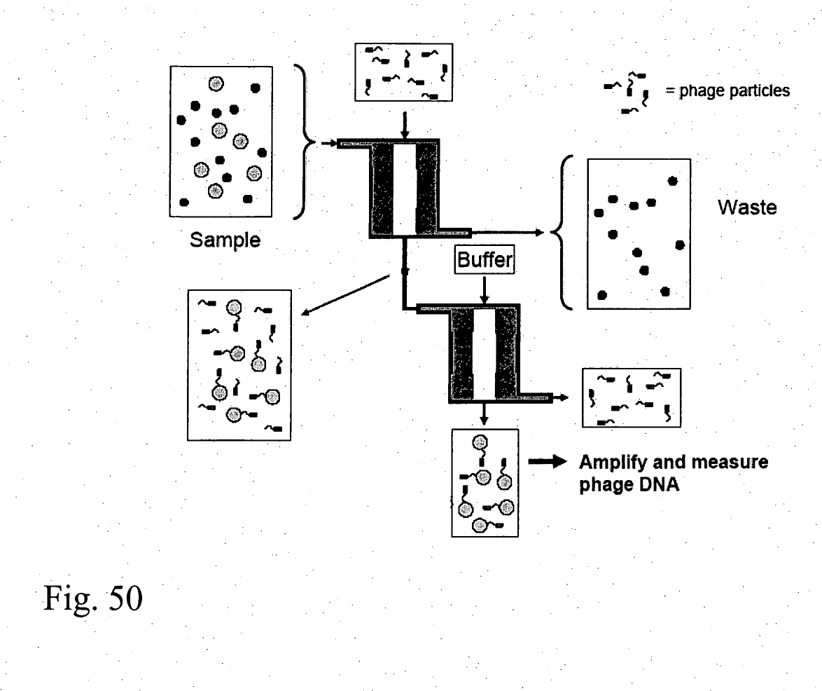

FIG. 50 depicts the use of phage as a labeling reagent in Which two device stages are arrayed in a cascade configuration. The method depicted in FIG. 50 fits the general description of FIG. 49, with the exception of the labeling reagent employed.

Desirably, downstream analysis results in an accurate determination of the number of target cells in the sample being analyzed. In order to produce accurate quantitative results, the surface antigen being targeted on the cells of interest typically has known or predictable expression levels, and the binding of the labeling reagent should also proceed in a predictable manner, free from interfering substances. Thus, methods of the invention that result in highly enriched cellular samples prior to introduction of labeling reagent are particularly useful. In addition, labeling reagents that allow for amplification of the signal produced are preferred, because of the low incidence of target cells, such as epithelial cells, e.g., CTCs, in the bloodstream. Reagents that allow for signal amplification include enzymes and phage. Other labeling reagents that do not allow for convenient amplification but nevertheless produce a strong signal, such as quantum dots, are also desirable.

It is not necessary to include a labeling reagent in the methods of the invention. For example, one method includes the steps of introducing a cellular sample, e.g., a sample of peripheral blood, into a device of the invention. For example, the device enriches cells having a hydrodynamic size greater than 12 microns, 14 microns, 16 microns, 18 microns, or even 20 microns from smaller cells in the sample. Alternatively, the device may enrich cells having a hydrodynamic size greater than or equal to 6 microns and less than or equal to 12 microns, e.g., cells having a hydrodynamic size greater than or equal to 8 microns and less than or equal to 10 microns, from other cells. The device may also enrich cells having a hydrodynamic size greater than or equal to 5 microns and less than or equal to 10 microns from cells having a hydrodynamic size greater than 10 microns; alternatively, it may enrich cells having a hydrodynamic size greater than or equal to 4 microns and less than or equal to 8 microns from cells having a hydrodynamic size greater than 8 microns. Each of these subsets of cells may then be collected and analyzed, e.g., by detecting the presence of a particular cell type, e.g., a rare cell, e.g., an epithelial cell or progenitor endothelial cell, in one of the samples thus collected. Because of the enrichment that this method generally achieves, the concentration of rare cells may be higher in a recovered sample than in the starting cellular sample, allowing for rare cell detection by a variety of means. In one embodiment, the cellular sample is applied to an inlet of the device; a second reagent, e.g., a buffer, e.g., a buffer containing BSA, a lysis reagent, a nucleic acid amplification reagent, an osmolarity regulating reagent, a labeling reagent, a preservative, or a fixing reagent, is optionally applied to a second inlet; and two output samples flow out of two outlets of the device. For example, application of a cellular sample containing cancer cells to an inlet of the device could result in one output sample that is enriched in such cells, while the other sample is depleted in these cells or even completely devoid of them. Any of the second reagents listed above may be employed in any of the devices and methods of the invention, e.g., those in which the device contains a second inlet.

In embodiments in which two cell types are directed in different directions, the first cell type being the cell type of interest, the second cell type may be any other cell type. For example, the second cell type may include white blood cells or red blood cells, e.g., enucleated red blood cells.

The methods of the invention need not employ either magnetic particles or interaction with an antibody or fragment thereof in order to enrich cells of interest, e.g., cancer cells, from a cellular sample. Any method based on cell size, shape, or deformability may be used in order to enrich cells of interest; subsequently, cell detection or any other downstream applications, e.g., those described herein, may be performed.

The methods of the invention allow for enrichment, quantification, and molecular biology analysis of the same set of cells. The gentle treatment of the cells in the devices of the invention, coupled with the described methods of bulk measurement, maintain the integrity of the cells so that further analysis may be performed if desired. For example, techniques that destroy the integrity of the cells may be performed subsequent to bulk measurement; such techniques include DNA or RNA analysis, proteome analysis, or metabolome analysis. For example, the total amount of DNA or RNA in a sample may be determined; alternatively, the presence of a particular sequence or mutation, e.g., a deletion, in DNA or RNA may be detected, e.g., a mutation in a gene encoding a polypeptide listed in Table 1. Furthermore, mitochondrial DNA, telomerase, or nuclear matrix proteins in the sample may be analyzed (for mitochondrial mutations in cancer, see, e.g., Parrella et al., Cancer Res. 61:7623-7626 (2001), Jones et al., Cancer Res. 61:1299-1304 (2001), and Fliss et al., Science 287:2017-2019 (2000); for telomerase, see, e.g., Soria et al., Clin. Cancer Res. 5:971-975 (1999)). For example, the sample may be analyzed to determine whether any mitochondrial abnormalities (see, e.g., Carew et al., Mol. Cancer 1:9 (2002), and Wallace, Science 283:1482-1488 (1999)) or perinuclear compartments are present. One useful method for analyzing DNA is PCR, in which the cells are lysed and levels of particular DNA sequences are amplified. Such techniques are particularly useful when the number of target cells isolated is very low. In-cell PCR may be employed; in addition, gene expression analysis (see, e.g., Giordano et al., Am. J. Pathol. 159:1231-1238 (2001), and Buckhaults et al., Cancer Res. 63:4144-4149 (2003)) or fluorescence in-situ hybridization may be used, e.g., to determine the tissue or tissues of origin of the cells being analyzed. A variety of cellular characteristics may be measured using any of the above techniques, such as protein phosphorylation, protein glycosylation, DNA methylation (see, e.g., Das et al., J. Clin. Oncol. 22:4632-4642 (2004)), microRNA levels (see, e.g., He et al., Nature 435:828-833 (2005), Lu et al., Nature 435:834-838 (2005), O'Donnell et al., Nature 435:839-843 (2005), and Calin et al., N. Engl. J. Med. 353:1793-1801 (2005)), cell morphology or other structural characteristics, e.g., pleomorphisms, adhesion, migration, binding, division, level of gene expression, and presence of a somatic mutation. This analysis may be performed on any number of cells, including a single cell of interest, e.g., a cancer cell. In addition, the size distribution of cells may be analyzed.

Desirably, downstream analysis, e.g., detection, is performed on more than one sample, preferably from the same subject.

Quantification of Cells

Cells found in blood are of various types and span a range of sizes. Using the methods of the invention, it is possible to distinguish, size, and count blood cell populations, e.g., CTCs. For example, a Coulter counter may be used. FIG. 33A shows a typical size distribution for a normal blood sample. Under some conditions, e.g., the presence of a tumor in the body that is exfoliating tumor cells, cells that are not native to blood may appear in the peripheral circulation. The ability to isolate and count large cells, or other desired cells, that may appear in the blood provides powerful opportunities for diagnosing disease states.

Desirably, a Coulter counter, or other cell detector, is fluidically coupled to an outlet of a device of the invention, and a cellular sample is introduced to the device of the invention. Cells flowing through the outlet fluidically coupled to the Coulter counter then pass through the Coulter aperture, which includes two electrodes separated by an opening through which the cells pass, and which measures the volume displaced as each cell passes through the opening. Preferably, the Coulter counter determines the number of cells of cell volume greater than 500 fL in the enriched sample. Alternatively, the Coulter counter preferably determines the number of cells of diameter greater than 14 μm in the enriched sample. The Coulter counter, or other cell detector, may also be an integral part of a device of the invention rather than constituting a separate device. The counter may utilize any cellular characteristic, e.g., impedance, light absorption, light scattering, or capacitance.

In general, any means of generating a cell count is useful in the methods of the invention. Such means include optical, such as scattering, absorption, or fluorescence means. Alternatively, non-aperture electrical means, such as determining capacitance, are useful.

Combination with Other Enrichment Techniques

Enrichment and alteration methods employing devices of the invention may be combined with other particulate sample manipulation techniques. In particular, further enrichment or purification of CTCs or other particles may be desirable. Further enrichment may occur by any technique, including affinity enrichment. Suitable affinity enrichment techniques include contacting particles of interest with affinity agents bound to channel walls or an array of obstacles. Such affinity agents may be selective for any cell type, e.g., cancer cells. This includes using a device of the invention in which antibodies specific for target cells are immobilized within the device. This allows for binding and enrichment of target cells within the device; subsequently the target cells are eluted using a higher flow rate, competing ligands, or another method.

Diagnosis

As described herein, epithelial cells exfoliated from solid tumors have been found in the circulation of patients with cancers of the breast, colon, liver, ovary, prostate, and lung. In general, the presence of CTCs after therapy has been associated with tumor progression and spread, poor response to therapy, relapse of disease, and/or decreased survival over a period of several years. Therefore, enumeration of CTCs offers a means to stratify patients for baseline characteristics that predict initial risk and subsequent risk based upon response to therapy.

The devices and methods of the invention may be used, e.g., to evaluate cancer patients and those at risk for cancer. In any of the methods of diagnosis described herein, either the presence or the absence of an indicator of cancer, e.g., a cancer cell, or of any other disorder, may be used to generate a diagnosis. In one example, a blood sample is drawn from the patient and introduced to a device of the invention with a critical size chosen appropriately to enrich epithelial cells, e.g., CTCs, from other blood cells. Using a method of the invention, the number of epithelial cells in the blood sample is determined. For example, the cells may be labeled with an antibody that binds to EpCAM, and the antibody may have a covalently bound fluorescent label. A bulk measurement may then be made of the enriched sample produced by the device, and from this measurement, the number of epithelial cells present in the initial blood sample may be determined. Microscopic techniques may be used to visually quantify the cells in order to correlate the bulk measurement with the corresponding number of labeled cells in the blood sample.

Besides epithelial tumor cells, there are other cell types that are involved in metastatic tumor formation. Studies have provided evidence for the involvement of hematopoietic bone marrow progenitor cells and endothelial progenitor cells in metastasis (see, e.g., Kaplan et al., Nature 438:820-827 (2005), and Brugger et al., Blood 83:636-640 (1994)). The number of cells of a second cell type, e.g., hematopoietic bone marrow progenitor cells, e.g., progenitor endothelial cells, may be determined, and the ratio of epithelial tumor cells to the number of the second cell type may be calculated. Such ratios are of diagnostic value in selecting the appropriate therapy and in monitoring the efficacy of treatment.

Cells involved in metastatic tumor formation may be detected using any methods known in the art. For example, antibodies specific for particular cell surface markers may be used. Useful endothelial cell surface markers include CD105, CD106, CD144, and CD146; useful tumor endothelial cell surface markers include TEM1, TEM5, and TEM8 (see, e.g., Carson-Walter et al., Cancer Res. 61:6649-6655 (2001)); and useful mesenchymal cell surface markers include CD133. Antibodies to these or other markers may be obtained from, e.g., Chemicon, Abcam, and R&D Systems.

By making a series of measurements, optionally made at regular intervals such as one day, two days, three days, one week, two weeks, one month, two months, three months, six months, or one year, one may track the level of epithelial cells present in a patient's bloodstream as a function of time. In the case of existing cancer patients, this provides a useful indication of the progression of the disease and assists medical practitioners in making appropriate therapeutic choices based on the increase, decrease, or lack of change in epithelial cells, e.g., CTCs, in the patient's bloodstream. For those at risk of cancer, a sudden increase in the number of cells detected may provide an early warning that the patient has developed a tumor. This early diagnosis, coupled with subsequent therapeutic intervention, is likely to result in an improved patient outcome in comparison to an absence of diagnostic information.

Diagnostic methods include making bulk measurements of labeled epithelial cells, e.g., CTCs, isolated from blood, as well as techniques that destroy the integrity of the cells. For example, PCR may be performed on a sample in which the number of target cells isolated is very low; by using primers specific for particular cancer markers, information may be gained about the type of tumor from which the analyzed cells originated. Additionally, RNA analysis, proteome analysis, or metabolome analysis may be performed as a means of diagnosing the type or types of cancer present in the patient.

One important diagnostic indicator for lung cancer and other cancers is the presence or absence of certain mutations in EGFR (see, e.g., International Publication WO 2005/094357). EGFR consists of an extracellular ligand-binding domain, a transmembrane portion, and an intracellular tyrosine kinase (TK) domain. The normal physiologic role of EGFR is to bind ErbB ligands, including epidermal growth factor (EGF), at the extracellular binding site to trigger a cascade of downstream intracellular signals leading to cell proliferation, survival, motility and other related activities. Many non-small cell lung tumors with EGFR mutations respond to small molecule EGFR inhibitors, such as gefitinib (Iressa; AstraZeneca), but often eventually acquire secondary mutations that make them drug resistant. Using the devices and method of the invention, one may monitor patients taking such drugs by taking frequent samples of blood and determining the number of epithelial cells, e.g., CTCs, in each sample as a function of time. This provides information as to the course of the disease. For example, a decreasing number of circulating epithelial cells over time suggests a decrease in the severity of the disease and the size of the tumor or tumors. Immediately following quantification of epithelial cells, these cells may be analyzed by PCR to determine what mutations may be present in the EFGR gene expressed in the epithelial cells. Certain mutations, such as those clustered around the ATP-binding pocket of the EGFR TK domain, are known to make the cancer cells susceptible to gefitinib inhibition. Thus, the presence of these mutations supports a diagnosis of cancer that is likely to respond to treatment using gefitinib. However, many patients who respond to gefitinib eventually develop a second mutation, often a methionine-to-threonine substitution at position 790 in exon 20 of the TK domain, which renders them resistant to gefitinib. By using the devices and method of the invention, one may test for this mutation as well, providing further diagnostic information about the course of the disease and the likelihood that it will respond to gefitinib or similar compounds. Since many EGFR mutations, including all EGFR mutations in NSC lung cancer reported to date that are known to confer sensitivity or resistance to gefitinib, lie within the coding regions of exons 18 to 21, this region of the EGFR gene may be emphasized in the development of assays for the presence of mutations (see Examples 4-6).

The methods of the invention described above are not limited to epithelial cells and cancer, but rather may be used to diagnose any condition. Exemplary conditions that may be diagnosed using the methods of the invention are hematological conditions, inflammatory conditions, ischemic conditions, neoplastic conditions, infections, traumas, endometriosis, and kidney failure (see, e.g., Takahashi et al., Nature Med. 5:434-438 (1999), Healy et al., Hum. Reprod. Update 4:736-740 (1998), and Gill et al., Circ. Res. 88:167-174 (2001)). Neoplastic conditions include acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor. In one embodiment, neoplastic cells associated with thyroid cancer are not detected. A cellular sample taken from a patient, e.g., a sample of less than 50 mL, 40 mL, 30 mL, 20 mL, or even 10 mL, may be processed through a device of the invention in order to produce a sample enriched in any cell of interest, e.g., a rare cell. Detection of this cell in the enriched sample may then enable one skilled in the art to diagnose the presence or absence of a particular condition in the patient. Furthermore, determination of ratios of numbers of cells, e.g., cancer cells to endothelial cells, in the sample may be used to generate a diagnosis. Alternatively, detection of cancer biomarkers, e.g., any of those listed in Table 1, or a nucleic acid associated with cancer, e.g., a nucleic acid encoding any marker listed in Table 1, may result in the diagnosis of a cancer or another condition. For example, analysis of the expression level or pattern of such a polypeptide or nucleic acid, e.g., cell surface markers, genomic DNA, mRNA, or microRNA, may result in a diagnosis.

Cell detection may be combined with other information, e.g., imaging studies of the patient, in order to diagnose a patient. For example, computed axial tomography, positron emission tomography, or magnetic resonance imaging may be used.

A diagnosis may also be made using a cell pattern associated with a particular condition. For example, by comparing the size distribution of cells in an enriched sample, e.g., a sample containing cells having a hydrodynamic size greater than 12 microns, with a size distribution associated with a condition, e.g., cancer, a diagnosis may be made based on this comparison. A cell pattern for comparison may be generated by any method. For example, an association study may be performed in which cellular samples from a plurality of control subjects (e.g., 50) and a plurality of case subjects (e.g., 50) having a condition of interest are processed, e.g., by enriching cells having a hydrodynamic size greater than 12 microns, the results samples are analyzed, and the results of the analysis are compared. To perform such a study, it may be useful to analyze RNA levels, e.g., mRNA or microRNA levels, in the enriched cells. Alternatively, it is useful to count the number of cells enriched in each case, or to determine a cellular size distribution, e.g., by using a microscope, a cell counter, or a microarray device. The presence of particular cell types, e.g., rare cells, may also be identified.

Once a drug treatment is administered to a patient, it is possible to determine the efficacy of the drug treatment using the methods of the invention. For example, a cellular sample taken from the patient before the drug treatment, as well as one or more cellular samples taken from the patient concurrently with or subsequent to the drug treatment, may be processed using the methods of the invention. By comparing the results of the analysis of each processed sample, one may determine the efficacy of the drug treatment. For example, an enrichment device may be used to enrich cells having a hydrodynamic size greater than 12 microns, or cells having a hydrodynamic size greater than or equal to 6 microns and less than or equal to 12 microns, from other cells. Any other detection or analysis described above may be performed, e.g., identification of the presence or quantity of specific cell types.

Methods of Using Sample Mobilization Devices

A sample mobilization device of the invention may be used to enrich CTCs or other cells from a sample. In one embodiment, a cellular sample is placed in a sample mobilization device, e.g., a device that includes a receptacle, a lid with a functionalized surface, and a sample mobilizer. The receptacle containing the sample is then covered with the lid, the sample mobilizer is employed to mobilize the sample, and the lid is removed. Such a device may be used to enrich a CTC or other cell of interest.

Any type of sample mobilization, e.g., centrifugation, may be applied. Any centrifugal field that is known in the art may be applied, e.g., a centrifugal field between 10 g and 100,000 g. For example, the centrifugal field may be between 1,000 g and 10,000 g. The application of this field results in a centrifugal force on the sample. Additional forces may also be applied, e.g., a force opposite to the centrifugal force; furthermore, forces may be applied repeatedly and in alternation, with an optional time interval between applications of each force.

General Considerations

Samples may be employed in the methods described herein with or without purification, e.g., stabilization and removal of certain components. Some sample may be diluted or concentrated prior to introduction into the device.

In one embodiment, reagents are added to the sample, to selectively or nonselectively increase the hydrodynamic size of the particles within the sample. This modified sample is then pumped through an obstacle array. Because the particles are swollen and have an increased hydrodynamic size, it will be possible to use obstacle arrays with larger and more easily manufactured gap sizes. In a preferred embodiment, the steps of swelling and size-based enrichment are performed in an integrated fashion on a device. Suitable reagents include any hypotonic solution, e.g., deionized water, 2% sugar solution, or neat non-aqueous solvents. Other reagents include beads, e.g., magnetic or polymer, that bind selectively (e.g., through antibodies or avidin-biotin) or non-selectively.

In another embodiment, reagents are added to the sample to selectively or nonselectively decrease the hydrodynamic size of the particles within the sample. Nonuniform decrease in particles in a sample will increase the difference in hydrodynamic size between particles. For example, nucleated cells are separated from enucleated cells by hypertonically shrinking the cells. The enucleated cells may shrink to a very small particle, while the nucleated cells cannot shrink below the size of the nucleus. Exemplary shrinking reagents include hypertonic solutions.

In an alternative embodiment, affinity functionalized beads and other appropriate beads are used to increase the volume of particles of interest relative to the other particles present in a sample, thereby allowing for the operation of a obstacle array with a larger and more easily manufactured gap size.

Fluids may be driven through a device either actively or passively. Fluids may be pumped using electric field, a centrifugal field, pressure-driven fluid flow, an electro-osmotic flow, and capillary action. In preferred embodiments, the average direction of the field will be parallel to the walls of the channel that contains the array.

Sample Preparation

Samples may be employed in the methods described herein with or without manipulation, e.g., stabilization and removal of certain components. In one embodiment, the sample is enriched in CTCs or other cells of interest prior to introduction to a device of the invention. Methods for enriching cell populations are known in the art, e.g., affinity mechanisms, agglutination, and size, shape, and deformability based enrichments. Exemplary methods for enriching a sample in a cell of interest are found in U.S. Pat. Nos. 5,837,115 and 5,641,628, International Publications WO 2004/029221 and WO 2004/113877, and U.S. Application Publication 2004/0144651.

### EXAMPLES

### Example 1

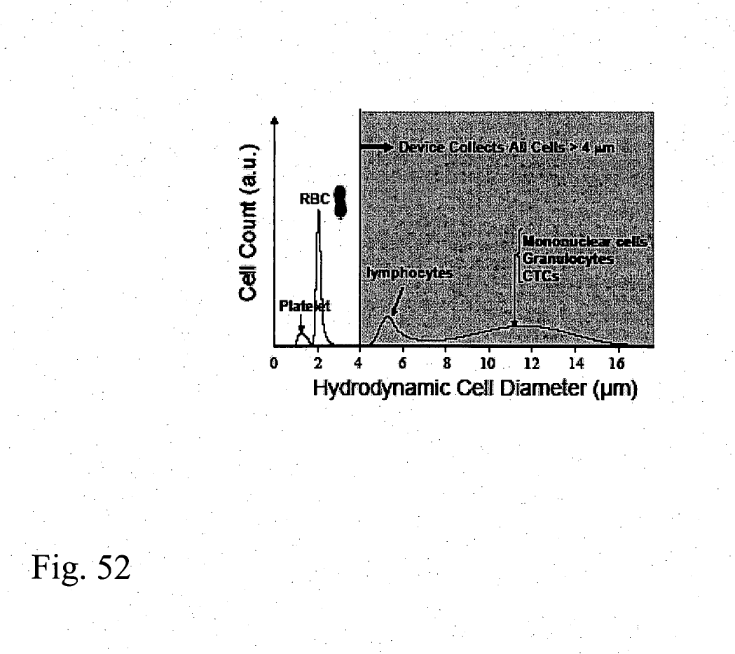

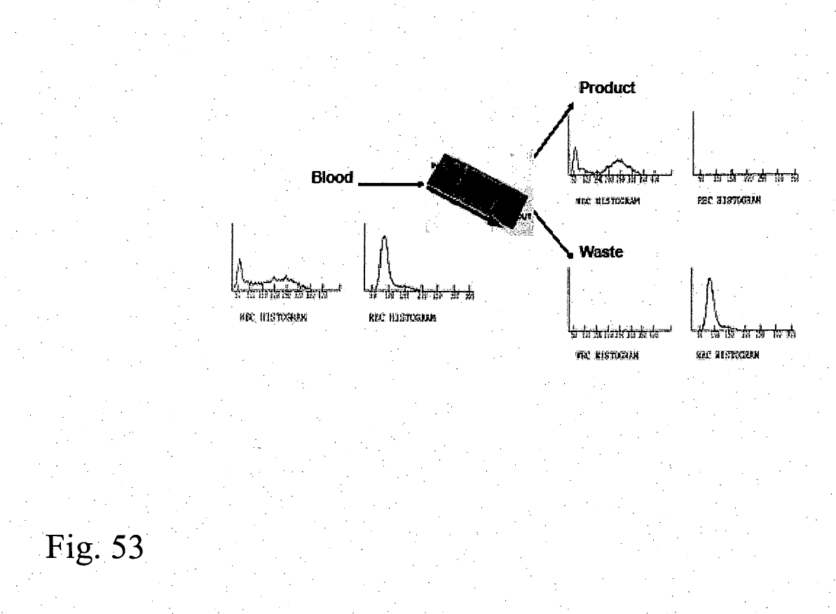

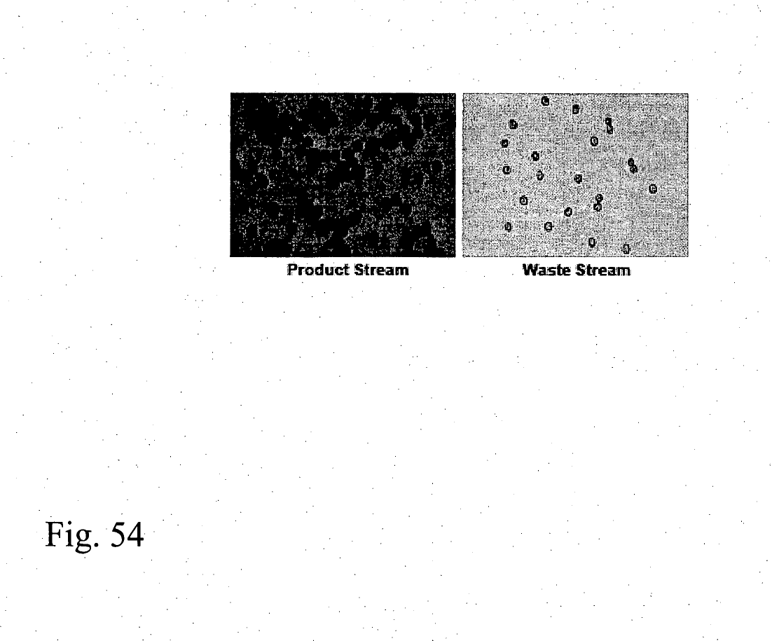

Microfluidic devices of the invention were designed by computer-aided design (CAD) and microfabricated by photolithography. A two-step process was developed in which a blood sample is first debulked to remove the large population of small cells, and then the rare target epithelial cells target cells are recovered by immunoaffinity capture. The devices were defined by photolithography and etched into a silicon substrate based on the CAD-generated design. The cell enrichment module, which is approximately the size of a standard microscope slide, contains 14 parallel sample processing sections and associated sample handling channels that connect to common sample and buffer inlets and product and waste outlets. Each section contains an array of microfabricated obstacles that is optimized to enrich the target cell type by hydrodynamic size via displacement of the larger cells into the product stream. In this example, the microchip was designed to separate red blood cells (RBCs) and platelets from the larger leukocytes and CTCs. Enriched populations of target cells were recovered from whole blood passed through the device. Performance of the cell enrichment microchip was evaluated by separating RBCs and platelets from white blood cells (WBCs) in normal whole blood (FIG. 52). In cancer patients, CTCs are found in the larger WBC fraction. Blood was minimally diluted (30%), and a 6 ml sample was processed at a flow rate of up to 6 ml/hr. The product and waste stream were evaluated in a Coulter Model “A^{C}-T diff” clinical blood analyzer, which automatically distinguishes, sizes, and counts different blood cell populations. The enrichment chip achieved separation of RBCs from WBCs, in which the WBC fraction had >99% retention of nucleated cells, >99% depletion of RBCs, and >97% depletion of platelets. Representative histograms of these cell fractions are shown in FIG. 53. Routine cytology confirmed the high degree of enrichment of the WBC and RBC fractions (FIG. 54).

Next, epithelial cells were recovered by affinity capture in a microfluidic module that is functionalized with immobilized antibody. A capture module with a single chamber containing a regular array of antibody-coated microfabricated obstacles was designed. These obstacles are disposed to maximize cell capture by increasing the capture area approximately four-fold, and by slowing the flow of cells under laminar flow adjacent to the obstacles to increase the contact time between the cells and the immobilized antibody. The capture modules may be operated under conditions of relatively high flow rate but low shear to protect cells against damage. The surface of the capture module was functionalized by sequential treatment with 10% silane, 0.5% gluteraldehyde, and avidin, followed by biotinylated anti-EpCAM. Active sites were blocked with 3% bovine serum albumin in PBS, quenched with dilute Tris HCl, and stabilized with dilute L-histidine. Modules were washed in PBS after each stage and finally dried and stored at room temperature. Capture performance was measured with the human advanced lung cancer cell line NCI-H1650 (ATCC Number CRL-5883). This cell line has a heterozygous 15 bp in-frame deletion in exon 19 of EGFR that renders it susceptible to gefitinib. Cells from confluent cultures were harvested with trypsin, stained with the vital dye Cell Tracker Orange (CMRA reagent, Molecular Probes, Eugene, Oreg.), resuspended in fresh whole blood, and fractionated in the microfluidic chip at various flow rates. In these initial feasibility experiments, cell suspensions were processed directly in the capture modules without prior fractionation in the cell enrichment module to debulk the red blood cells; hence, the sample stream contained normal blood red cells and leukocytes as well as tumor cells. After the cells were processed in the capture module, the device was washed with buffer at a higher flow rate (3 ml/hr) to remove the nonspecifically bound cells. The adhesive top was removed and the adherent cells were fixed on the chip with paraformaldehyde and observed by fluorescence microscopy. Cell recovery was calculated from hemacytometer counts; representative capture results are shown in Table 2. Initial yields in reconstitution studies with unfractionated blood were greater than 60% with less than 5% of non-specific binding.

TABLE 2

| Run | Avg. flow | Length of | No. cells | No. cells | |
|---|---|---|---|---|---|
| number | rate | run | processed | captured | Yield |
| 1 | 3.0 | 1 hr | 150,000 | 38,012 | 25% |
| 2 | 1.5 | 2 hr | 150,000 | 30,000/ml | 60% |
| 3 | 1.08 | 2 hr | 108,000 | 68,661 | 64% |
| 4 | 1.21 | 2 hr | 121,000 | 75,491 | 62% |

Next, NCI-H1650 cells that were spiked into whole blood and recovered by size fractionation and affinity capture as described above were successfully analyzed in situ. In a trial run to distinguish epithelial cells from leukocytes, 0.5 ml of a stock solution of fluorescein-labeled CD45 pan-leukocyte monoclonal antibody were passed into the capture module and incubated at room temperature for 30 minutes. The module was washed with buffer to remove unbound antibody, and the cells were fixed on the chip with 1% paraformaldehyde and observed by fluorescence microscopy. As shown in FIG. 55, the epithelial cells were bound to the obstacles and floor of the capture module. Background staining of the flow passages with CD45 pan-leukocyte antibody is visible, as are several stained leukocytes, apparently because of a low level of non-specific capture.

### Example 2

### Device Embodiments

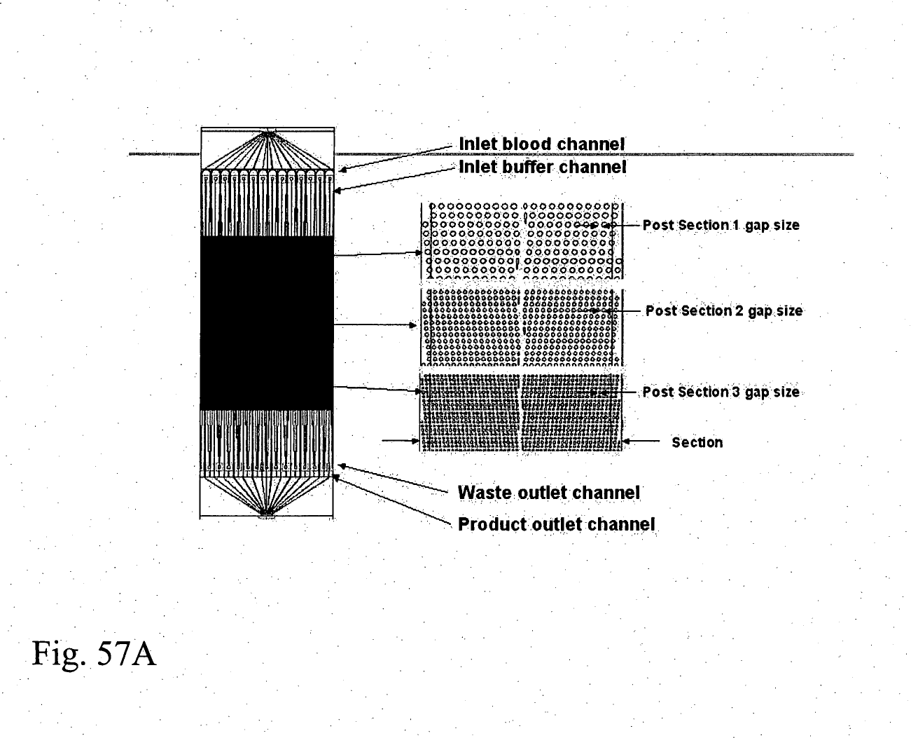

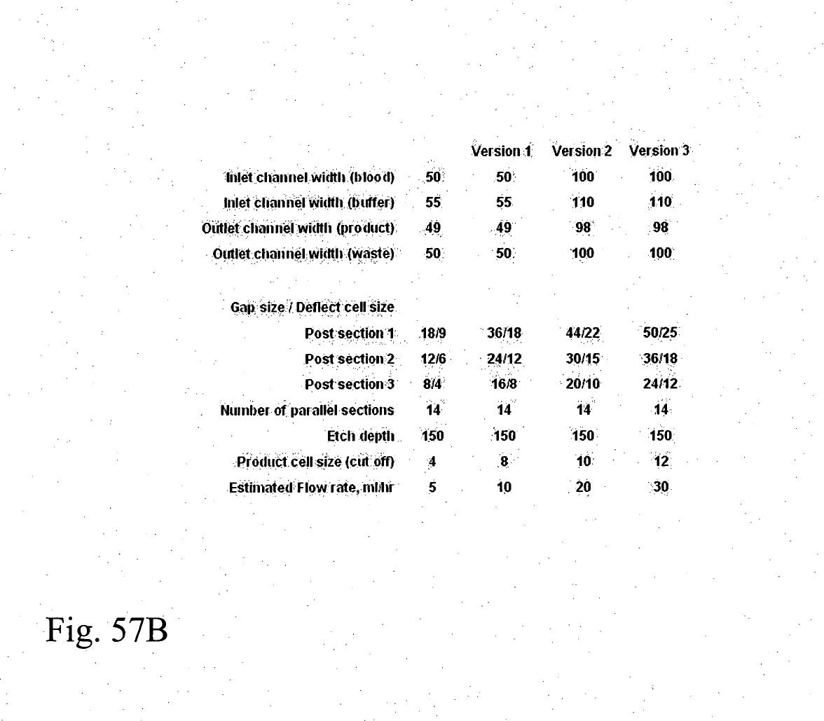

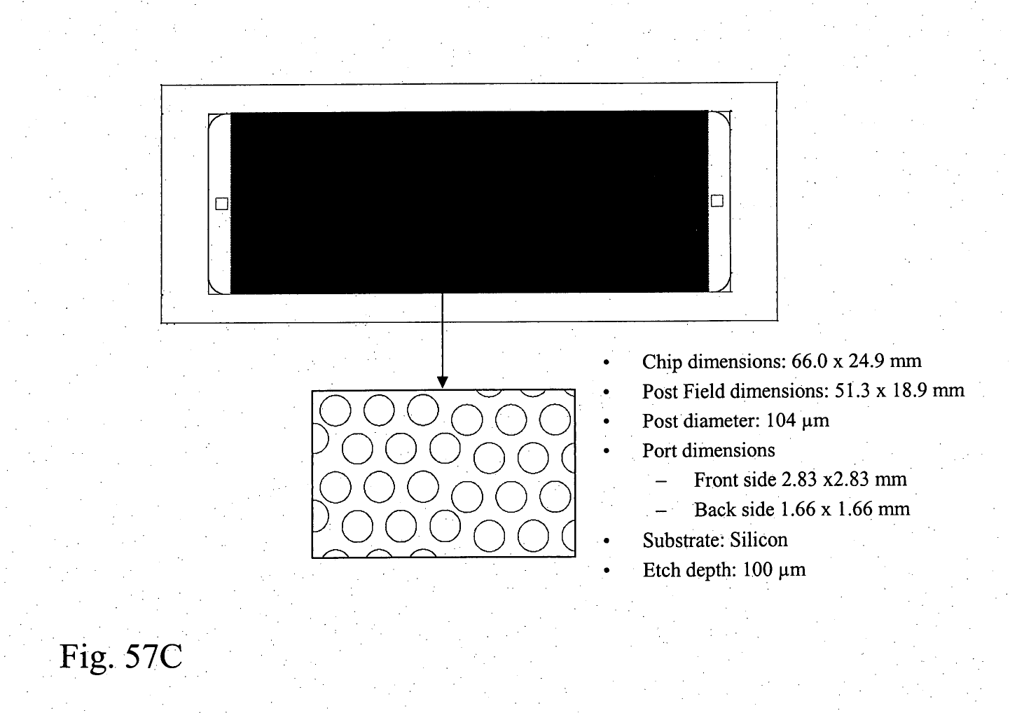

A design for preferred device embodiments of the invention is shown in FIG. 57A, and parameters corresponding to three preferred device embodiments associated with this design are shown in FIG. 57B. These embodiments are particularly useful for enrich epithelial cells from blood.

### Example 3

### Determining Counts for Non-Epithelial Cell Types

Using the methods of the invention, one may make a diagnosis based on counting cell types other than CTCs or other epithelial cells. A diagnosis of the absence, presence, or progression of cancer may be based on the number of cells in a cellular sample that are larger than a particular cutoff size. For example, cells with a hydrodynamic size of 14 microns or larger may be selected. This cutoff size would eliminate most leukocytes. The nature of these cells may then be determined by downstream molecular or cytological analysis.

Cell types other than epithelial cells that would be useful to analyze include endothelial cells, endothelial progenitor cells, endometrial cells, or trophoblasts indicative of a disease state. Furthermore, determining separate counts for epithelial cells, e.g., cancer cells, and other cell types, e.g., endothelial cells, followed by a determination of the ratios between the number of epithelial cells and the number of other cell types, may provide useful diagnostic information.

A device of the invention may be configured to isolate targeted subpopulations of cells such as those described above, as shown in FIGS. 33A-D. A size cutoff may be selected such that most native blood cells, including red blood cells, white blood cells, and platelets, flow to waste, while non-native cells, which could include endothelial cells, endothelial progenitor cells, endometrial cells, or trophoblasts, are collected in an enriched sample. This enriched sample may be further analyzed.

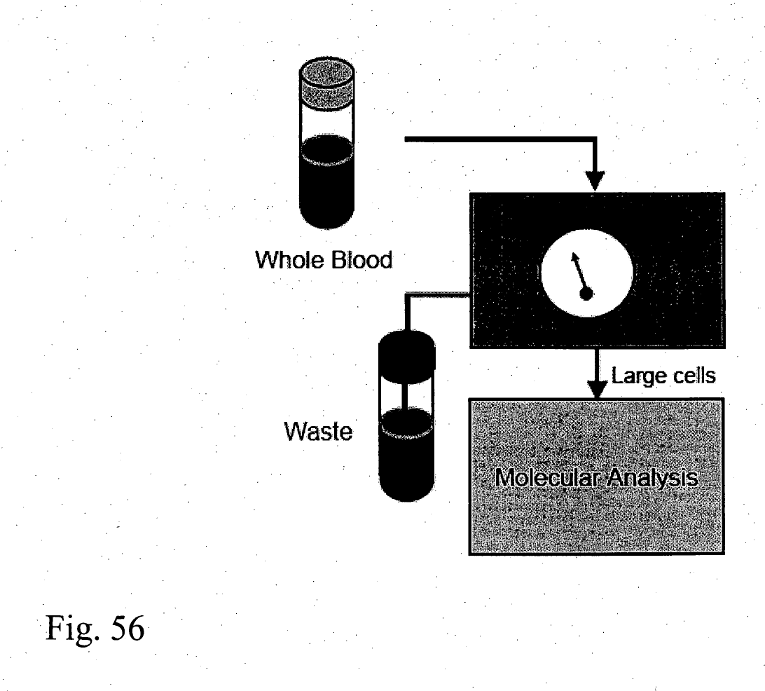

Using a device of the invention, therefore, it is possible to isolate a subpopulation of cells from blood or other bodily fluids based on size, which conveniently allows for the elimination of a large proportion of native blood cells when large cell types are targeted. As shown schematically in FIG. 56, a device of the invention may include counting means to determine the number of cells in the enriched sample, or the number of cells of a particular type, e.g., cancer cells, within the enriched sample, and further analysis of the cells in the enriched sample may provide additional information that is useful for diagnostic or other purposes.

### Example 4

### Method for Detection of EGFR Mutations

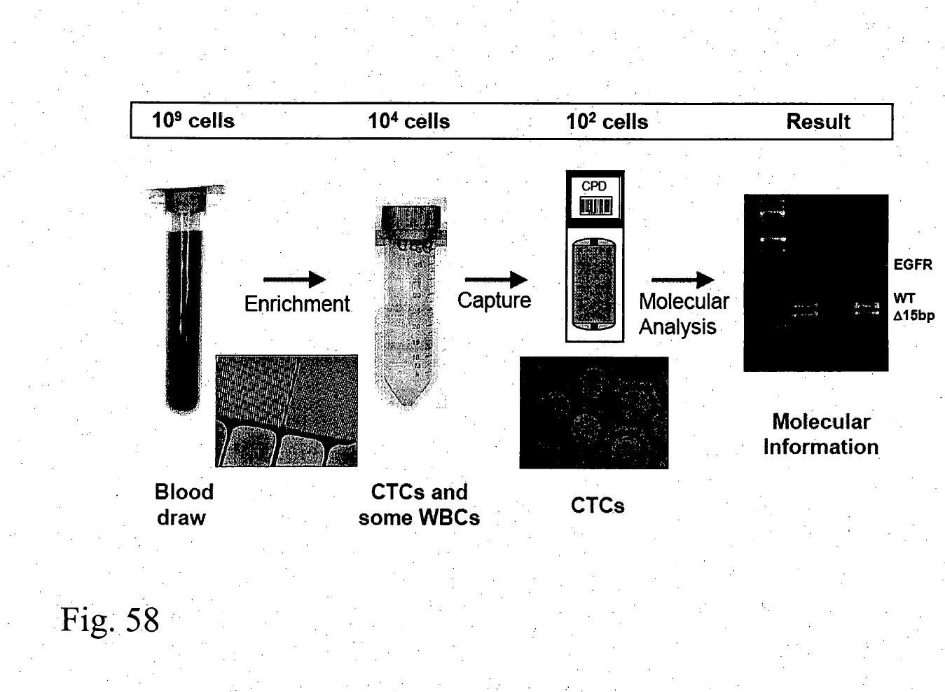

A blood sample from a cancer patient is processed and analyzed using the devices and methods of the invention, e.g., those of Example 1, resulting in an enriched sample of epithelial cells containing CTCs. This sample is then analyzed to identify potential EGFR mutations. The method permits both identification of known, clinically relevant EGFR mutations as well as discovery of novel mutations. An overview of this process is shown in FIG. 58.

Below is an outline of the strategy for detection and confirmation of EGFR mutations:

1) Sequence CTC EGFR mRNA
a) Purify CTCs from blood sample;
b) Purify total RNA from CTCs;
c) Convert RNA to cDNA using reverse transcriptase;
d) Use resultant cDNA to perform first and second PCR reactions for generating sequencing templates; and
e) Purify the nested PCR amplicon and use as a sequencing template to sequence EGFR exons 18-21.

2) Confirm RNA Sequence Using CTC Genomic DNA
a) Purify CTCs from blood sample;
b) Purify genomic DNA (gDNA) from CTCs;
c) Amplify exons 18, 19, 20, and/or 21 via PCR reactions; and
d) Use the resulting PCR amplicon(s) in real-time quantitative allele-specific PCR reactions in order to confirm the sequence of mutations discovered via RNA sequencing.

Further details for each step outlined above are as follows.

1) Sequence CTC EGFR mRNA

a) Purify CTCs from blood sample. CTCs are isolated using any of the size-based enrichment and/or affinity purification devices of the invention.

b) Purify total RNA from CTCs. Total RNA is then purified from isolated CTC populations using, e.g., the Qiagen Micro RNeasy kit, or a similar total RNA purification protocol from another manufacturer; alternatively, standard RNA purification protocols such as guanidium isothiocyanate homogenization followed by phenol/chloroform extraction and ethanol precipitation may be used. One such method is described in “Molecular Cloning—A Laboratory Manual, Second Edition” (1989) by J. Sambrook, E. F. Fritch and T. Maniatis, p. 7.24.

c) Convert RNA to cDNA using reverse transcriptase. cDNA reactions are carried out based on the protocols of the supplier of reverse transcriptase. Typically, the amount of input RNA into the cDNA reactions is in the range of 10 picograms (pg) to 2 micrograms (μg) total RNA. First-strand DNA synthesis is carried out by hybridizing random 7mer DNA primers, or oligo-dT primers, or gene-specific primers, to RNA templates at 65° C. followed by snap-chilling on ice. cDNA synthesis is initiated by the addition of iScript Reverse Transcriptase (BioRad) or SuperScript Reverse Transcriptase (Invitrogen) or a reverse transcriptase from another commercial vendor along with the appropriate enzyme reaction buffer. For iScript, reverse transcriptase reactions are carried out at 42° C. for 30-45 minutes, followed by enzyme inactivation for 5 minutes at 85° C. cDNA is stored at −20° C. until use or used immediately in PCR reactions. Typically, cDNA reactions are carried out in a final volume of 20 μl, and 10% (2 μl) of the resultant cDNA is used in subsequent PCR reactions.

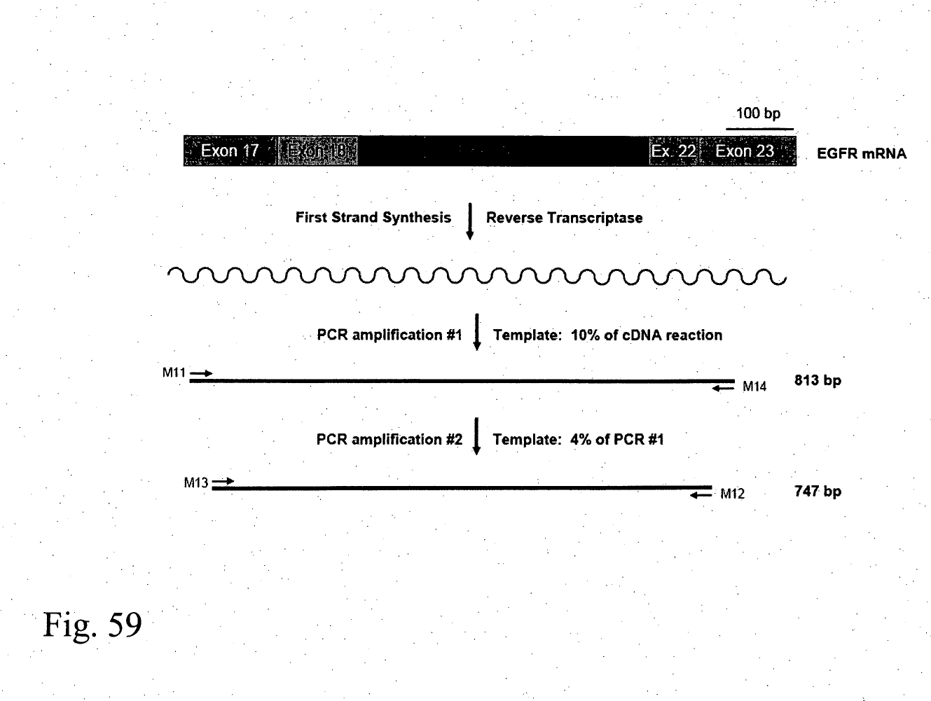

d) Use resultant cDNA to perform first and second PCR reactions for generating sequencing templates. cDNA from the reverse transcriptase reactions is mixed with DNA primers specific for the region of interest (FIG. 59). See Table 3 for sets of primers that may be used for amplification of exons 18-21. In Table 3, primer set M13(+)/M12(−) is internal to primer set M11(+)/M14(−). Thus primers M13(+) and M12(−) may be used in the nested round of amplification, if primers M11(+) and M14(−) were used in the first round of expansion. Similarly, primer set M11(+)/M14(−) is internal to primer set M15(+)/M16(−), and primer set M23(+)/M24(−) is internal to primer set M21(+)/M22(−). Hot Start PCR reactions are performed using Qiagen Hot-Star Taq Polymerase kit, or Applied Biosystems HotStart TaqMan polymerase, or other Hot Start thermostable polymerase, or without a hot start using Promega GoTaq Green Taq Polymerase master mix, TaqMan DNA polymerase, or other thermostable DNA polymerase. Typically, reaction volumes are 50 μl, nucleotide triphosphates are present at a final concentration of 200 μM for each nucleotide, MgCl₂ is present at a final concentration of 1-4 mM, and oligo primers are at a final concentration of 0.5 μM. Hot start protocols begin with a 10-15 minute incubation at 95° C., followed by 40 cycles of 94° C. for one minute (denaturation), 52° C. for one minute (annealing), and 72° C. for one minute (extension). A 10 minute terminal extension at 72° C. is performed before samples are stored at 4° C. until they are either used as template in the second (nested) round of PCRs, or purified using QiaQuick Spin Columns (Qiagen) prior to sequencing. If a hot-start protocol is not used, the initial incubation at 95° C. is omitted. If a PCR product is to be used in a second round of PCRs, 2 μl (4%) of the initial PCR product is used as template in the second round reactions, and the identical reagent concentrations and cycling parameters are used.

TABLE 3

Primer Sets for expanding EGFR mRNA around
Exons 18-21

| | | | | |
|---|---|---|---|---|
| | | | | Am- |
| | SEQ | | | pli- |
| | ID | | cDNA | con |
| Name | NO | Sequence (5′ to 3′) | Coordinates | Size |

| | | | | |
|---|---|---|---|---|
| NXK- | 1 | TTGCTGCTGGTGGTGGC | (+) 1966-1982 | 813 |
| M11(+) | | | | |
| NXK- | 2 | CAGGGATTCCGTCATATGGC | (−) 2778-2759 | |
| M14(−) | | | | |
| NXK- | 3 | GATCGGCCTCTTCATGCG | (+) 1989-2006 | 747 |
| M13(+) | | | | |
| NXK- | 4 | GATCCAAAGGTCATCAACTCCC | (−) 2735-2714 | |
| M12(−) | | | | |
| NXK- | 5 | GCTGTCCAACGAATGGGC | (+) 1904-1921 | 894 |
| M15(+) | | | | |
| NXK- | 6 | GGCGTTCTCCTTTCTCCAGG | (−) 2797-2778 | |
| M16(−) | | | | |
| NXK- | 7 | ATGCACTGGGCCAGGTCTT | (+) 1881-1899 | 944 |
| M21(+) | | | | |
| NXK- | 8 | CGATGGTACATATGGGTGGCT | (−) 2824-2804 | |
| M22(−) | | | | |
| NXK- | 9 | AGGCTGTCCAACGAATGGG | (+) 1902-1920 | 904 |
| M23(+) | | | | |
| NXK- | 10 | CTGAGGGAGGCGTTCTCCT | (−) 2805-2787 | |
| M24(−) | | | | |

e) Purify the nested PCR amplicon and use as a sequencing template to sequence EGFR exons 18-21. Sequencing is performed by ABI automated fluorescent sequencing machines and fluorescence-labeled DNA sequencing ladders generated via Sanger-style sequencing reactions using fluorescent dideoxynucleotide mixtures. PCR products are purified using Qiagen QuickSpin columns, the Agencourt AMPure PCR Purification System, or PCR product purification kits obtained from other vendors. After PCR products are purified, the nucleotide concentration and purity is determined with a Nanodrop 7000 spectrophotometer, and the PCR product concentration is brought to a concentration of 25 ng/μl. As a quality control measure, only PCR products that have a UV-light absorbance ratio (A₂₆₀/A₂₈₀) greater than 1.8 are used for sequencing. Sequencing primers are brought to a concentration of 3.2 pmol/μl.

2) Confirm RNA Sequence Using CTC Genomic DNA

a) Purify CTCs from blood sample. As above, CTCs are isolated using any of the size-based enrichment and/or affinity purification devices of the invention.

b) Purify genomic DNA (gDNA) from CTCs. Genomic DNA is purified using the Qiagen DNeasy Mini kit, the Invitrogen ChargeSwitch gDNA kit, or another commercial kit, or via the following protocol:

1. Cell pellets are either lysed fresh or stored at −80° C. and are thawed immediately before lysis.

2. Add 500 μl 50 mM Tris pH 7.9/100 mM EDTA/0.5% SDS (TES buffer).

3. Add 12.5 μl Proteinase K (IBI5406, 20 mg/ml), generating a final [ProtK]=0.5 mg/ml.

4. Incubate at 55° C. overnight in rotating incubator.

5. Add 20 μl of RNase cocktail (500 U/ml RNase A+20,000 U/ml RNase T1, Ambion #2288) and incubate four hours at 37° C.

6. Extract with Phenol (Kodak, Tris pH 8 equilibrated), shake to mix, spin 5 min. in tabletop centrifuge.

7. Transfer aqueous phase to fresh tube.

8. Extract with Phenol/Chloroform/Isoamyl alcohol (EMD, 25:24:1 ratio, Tris pH 8 equilibrated), shake to mix, spin five minutes in tabletop centrifuge.

9. Add 50 μl 3M NaOAc pH=6.

10. Add 500 μl EtOH.

11. Shake to mix. Strings of precipitated DNA may be visible. If anticipated DNA concentration is very low, add carrier nucleotide (usually yeast tRNA).

12. Spin one minute at max speed in tabletop centrifuge.

13. Remove supernatant.

14. Add 500 μl 70% EtOH, Room Temperature (RT)

15. Shake to mix.

16. Spin one minute at max speed in tabletop centrifuge.

17. Air dry 10-20 minutes before adding TE.

18. Resuspend in 400 μl TE. Incubate at 65° C. for 10 minutes, then leave at RT overnight before quantitation on Nanodrop.

c) Amplify exons 18, 19, 20, and/or 21 via PCR reactions. Hot start nested PCR amplification is carried out as described above in step 1d, except that there is no nested round of amplification. The initial PCR step may be stopped during the log phase in order to minimize possible loss of allele-specific information during amplification. The primer sets used for expansion of EGFR exons 18-21 are listed in Table 4 (see also Paez et al., Science 304:1497-1500 (Supplementary Material) (2004)).

TABLE 4

Primer sets for expanding EGFR genomic DNA

| | SEQ | | | |
|---|---|---|---|---|
| | ID | | | Amplicon |
| Name | NO | Sequence (5′ to 3′) | Exon | Size |
| NXK- | 11 | TCAGAGCCTGTGTTTCTACCAA | 18 | 534 |
| ex18.1(+) | | | | |
| NXK- | 12 | TGGTCTCACAGGACCACTGATT | 18 | |
| ex18.2(−) | | | | |
| NXK- | 13 | TCCAAATGAGCTGGCAAGTG | 18 | 397 |
| ex18.3(+) | | | | |
| NXK- | 14 | TCCCAAACACTCAGTGAAACAAA | 18 | |
| ex18.4(−) | | | | |
| NXK- | 15 | AAATAATCAGTGTGATTCGTGGAG | 19 | 495 |
| ex19.1(+) | | | | |
| NXK- | 16 | GAGGCCAGTGCTGTCTCTAAGG | 19 | |
| ex19.2(−) | | | | |
| NXK- | 17 | GTGCATCGCTGGTAACATCC | 19 | 298 |
| ex19.3(+) | | | | |
| NXK- | 18 | TGTGGAGATGAGCAGGGTCT | 19 | |
| ex19.4(−) | | | | |
| NXK- | 19 | ACTTCACAGCCCTGCGTAAAC | 20 | 555 |
| ex20.1(+) | | | | |
| NXK- | 20 | ATGGGACAGGCACTGATTTGT | 20 | |
| ex20.2(−) | | | | |
| NXK- | 21 | ATCGCATTCATGCGTCTTCA | 20 | 379 |
| ex20.3(+) | | | | |
| NXK- | 22 | ATCCCCATGGCAAACTCTTG | 20 | |
| ex20.4(−) | | | | |
| NXK- | 23 | GCAGCGGGTTACATCTTCTTTC | 21 | 526 |
| ex21.1(+) | | | | |
| NXK- | 24 | CAGCTCTGGCTCACACTACCAG | 21 | |
| ex21.2(−) | | | | |
| NXK- | 25 | GCAGCGGGTTACATCTTCTTTC | 21 | 349 |
| ex21.3(+) | | | | |
| NXK- | 26 | CATCCTCCCCTGCATGTGT | 21 | |
| ex21.4(−) | | | | |

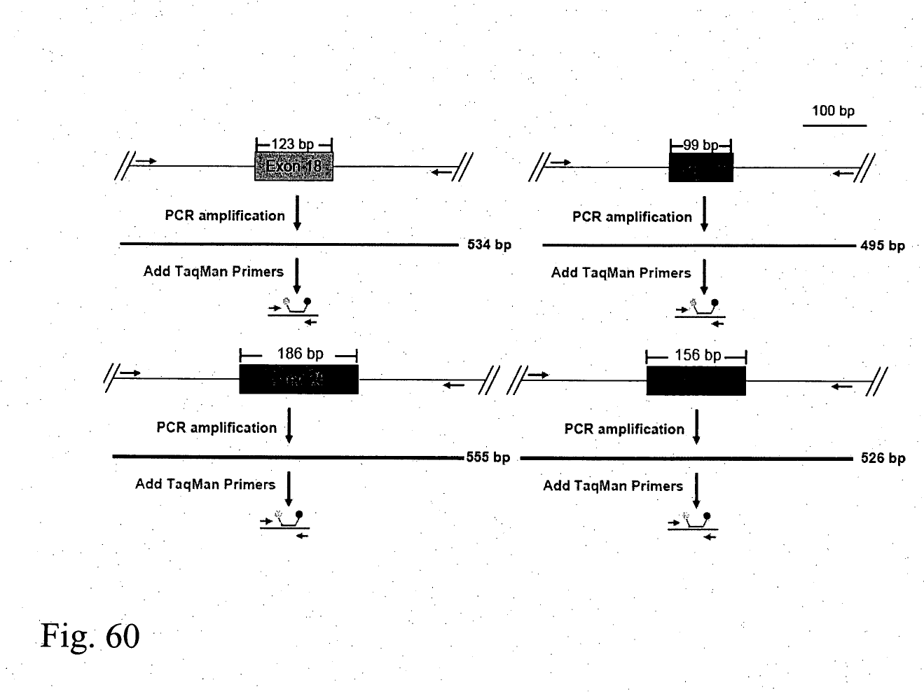

d) Use the resulting PCR amplicon(s) in real-time quantitative allele-specific PCR reactions in order to confirm the sequence of mutations discovered via RNA sequencing. An aliquot of the PCR amplicons is used as template in a multiplexed allele-specific quantitative PCR reaction using TaqMan PCR 5′ Nuclease assays with an Applied Biosystems model 7500 Real Time PCR machine (FIG. 60). This round of PCR amplifies subregions of the initial PCR product specific to each mutation of interest. Given the very high sensitivity of Real Time PCR, it is possible to obtain complete information on the mutation status of the EGFR gene even if as few as 10 CTCs are isolated. Real Time PCR provides quantification of allelic sequences over 8 logs of input DNA concentrations; thus, even heterozygous mutations in impure populations are easily detected using this method.

Probe and primer sets are designed for all known mutations that affect gefitinib responsiveness in NSCLC patients, including over 40 such somatic mutations, including point mutations, deletions, and insertions, that have been reported in the medical literature. For illustrative purposes, examples of primer and probe sets for five of the point mutations are listed in Table 5. In general, oligonucleotides may be designed using the primer optimization software program Primer Express (Applied Biosystems), with hybridization conditions optimized to distinguish the wild type EGFR DNA sequence from mutant alleles. EGFR genomic DNA amplified from lung cancer cell lines that are known to carry EGFR mutations, such as H358 (wild type), H1650 (15-bp deletion, Δ2235-2249), and H1975 (two point mutations, 2369 C→T, 2573 T→G), is used to optimize the allele-specific Real Time PCR reactions. Using the TaqMan 5′ nuclease assay, allele-specific labeled probes specific for wild type sequence or for known EGFR mutations are developed. The oligonucleotides are designed to have melting temperatures that easily distinguish a match from a mismatch, and the Real Time PCR conditions are optimized to distinguish wild type and mutant alleles. All Real Time PCR reactions are carried out in triplicate.

Initially, labeled probes containing wild type sequence are multiplexed in the same reaction with a single mutant probe. Expressing the results as a ratio of one mutant allele sequence versus wild type sequence may identify samples containing or lacking a given mutation. After conditions are optimized for a given probe set, it is then possible to multiplex probes for all of the mutant alleles within a given exon within the same Real Time PCR assay, increasing the ease of use of this analytical tool in clinical settings.

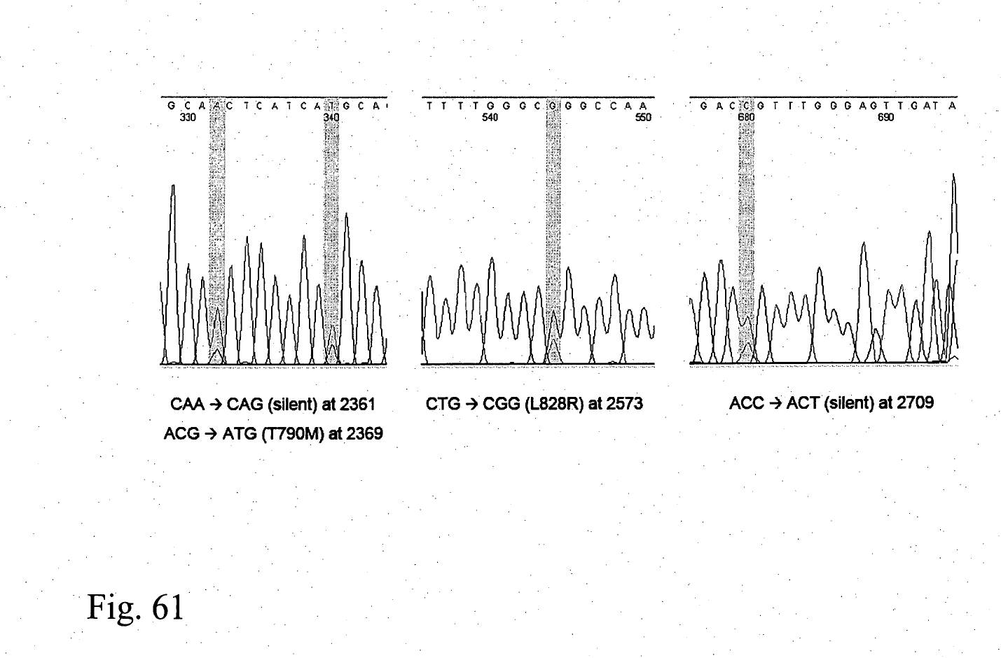

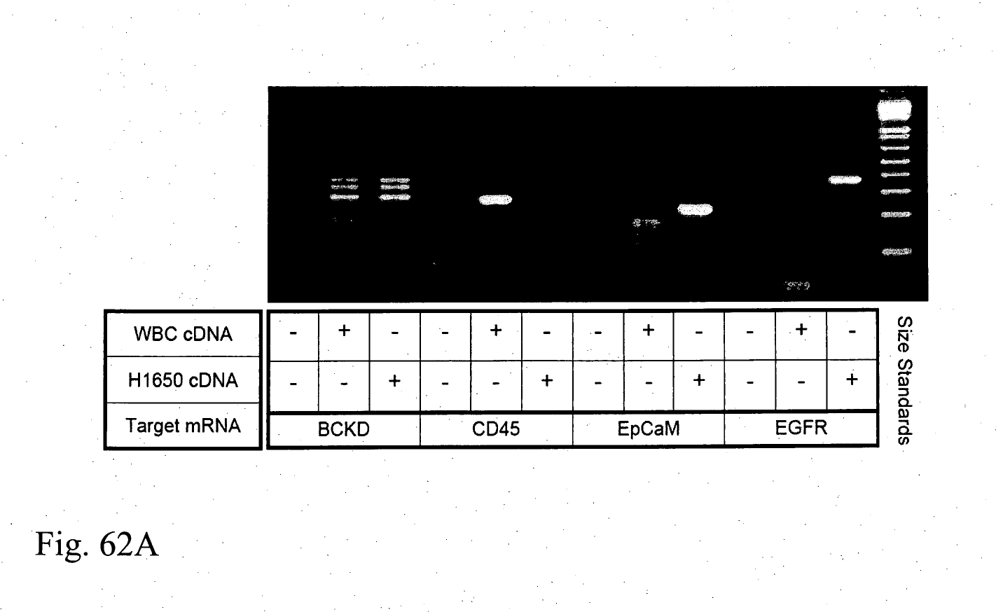

A unique probe is designed for each wild type allele and mutant allele sequence. Wild-type sequences are marked with the fluorescent dye VIC at the 5′ end, and mutant sequences with the fluorophore FAM. A fluorescence quencher and Minor Groove Binding moiety are attached to the 3′ ends of the probes. ROX is used as a passive reference dye for normalization purposes. A standard curve is generated for wild type sequences and is used for relative quantitation. Precise quantitation of mutant signal is not required, as the input cell population is of unknown, and varying, purity. The assay is set up as described by ABI product literature, and the presence of a mutation is confirmed when the signal from a mutant allele probe rises above the background level of fluorescence (FIG. 61), and this threshold cycle gives the relative frequency of the mutant allele in the input sample.

TABLE 5

Probes and Primers for Allele-Specific qPCR

| | SEQ | Sequence (5′ to 3′, | | | |
|---|---|---|---|---|---|
| | ID | mutated position | cDNA | | |
| Name | NO | in bold) | Coordinates | Description | Mutation |
| NXK-M01 | 27 | CCGCAGCATGTCAAGATCAC | (+)2542- | (+)primer | L858R |
| | | | 2561 | | |
| NXK-M02 | 28 | TCCTTCTGCATGGTATTCTTTCTCT | (−)2619- | (−)primer | |
| | | | 2595 | | |
| Pwt-L858R | 29 | VIC-TTTGGGCTGGCCAA-MGB | (+)2566- | WT allele | |
| | | | 2579 | probe | |
| Pmut- | 30 | FAM-TTTTGGGCGGGCCA-MGB | (+)2566- | Mutant | |
| L858R | | | 2579 | allele | |
| | | | | probe | |
| NXK-M03 | 31 | ATGGCCAGCGTGGACAA | (+)2296- | (+)primer | T790M |
| | | | 2312 | | |
| NXK-M04 | 32 | AGCAGGTACTGGGAGCCAATATT | (−)2444- | (−)primer | |
| | | | 2422 | | |
| Pwt- | 33 | VIC-ATGAGCTGCGTGATGA-MGB | (−)2378- | WT allele | |
| T790M | | | 2363 | probe | |
| Pmut- | 34 | FAM-ATGAGCTGCATGATGA-MGB | (−)2378- | Mutant | |
| T790M | | | 2363 | allele | |
| | | | | probe | |
| NXK-M05 | 35 | GCCTCTTACACCCAGTGGAGAA | (+)2070- | (+)primer | G719S,C |
| | | | 2091 | | |
| NXK-M06 | 36 | TTCTGGGATCCAGAGTCCCTTA | (−)2202- | (−)primer | |
| | | | 2181 | | |
| Pwt- | 37 | VIC-ACCGGAGCCCAGCA-MGB | (−)2163- | WT allele | |
| G719SC | | | 2150 | probe | |
| Pmut- | 38 | FAM-ACCGGAGCTCAGCA-MGB | (−)2163- | Mutant | |
| G719S | | | 2150 | allele | |
| | | | | probe | |
| Pmut- | 39 | FAM-ACCGGAGCACAGCA-MGB | (−)2163- | Mutant | |
| G719C | | | 2150 | allele | |
| | | | | probe | |
| NXK-M09 | 40 | TCGCAAAGGGCATGAACTACT | (+)2462- | (+)primer | H835L |
| | | | 2482 | | |
| NXK-M10 | 41 | ATCTTGACATGCTGCGGTGTT | (−)2558- | (−)primer | |
| | | | 2538 | | |
| Pwt-H835L | 42 | VIC-TTGGTGCACCGCGA-MGB | (+)2498- | WT allele | |
| | | | 2511 | probe | |
| Pmut- | 43 | FAM-TGGTGCTCCGCGAC-MGB | (+)2498- | Mutant | |
| H835L | | | 2511 | allele | |
| | | | | probe | |

### Example 5

### Absence of EGFR Expression in Leukocytes

The protocol of Example 4 would be most useful if EGFR were expressed in target cancer cells but not in background leukocytes. To test whether EGFR mRNA is present in leukocytes, several PCR experiments were performed. Four sets of primers, shown in Table 6, were designed to amplify four corresponding genes:

1) BCKDK (branched-chain a-ketoacid dehydrogenase complex kinase)—a “housekeeping” gene expressed in all types of cells, a positive control for both leukocytes and tumor cells;

2) CD45—specifically expressed in leukocytes, a positive control for leukocytes and a negative control for tumor cells;

3) EpCaM—specifically expressed in epithelial cells, a negative control for leukocytes and a positive control for tumor cells; and

4) EGFR—the target mRNA to be examined.

TABLE 6

| | SEQ | | | Ampli- |
|---|---|---|---|---|
| | ID | | Descrip- | con |
| Name | NO | Sequence (5′ to 3′) | tion | Size |
| BCKD_1 | 44 | AGTCAGGACCCATGCACGG | BCKDK (+) | 273 |
| | | | primer | |
| BCKD_2 | 45 | ACCCAAGATGCAGCAGTGTG | BCKDK (−) | |
| | | | primer | |
| CD_1 | 46 | GATGTCCTCCTTGTTCTACTC | CD45 (+) | 263 |
| | | | primer | |
| CD_2 | 47 | TACAGGGAATAATCGAGCATGC | CD45 (−) | |
| | | | primer | |
| EpCAM_1 | 48 | GAAGGGAAATAGCAAATGGACA | EpCAM (+) | 222 |
| | | | primer | |
| EpCAM_2 | 49 | CGATGGAGTCCAAGTTCTGG | EpCAM (−) | |
| | | | primer | |
| EGFR_1 | 50 | AGCACTTACAGCTCTGGCCA | EGFR (+) | 371 |
| | | | primer | |
| EGFR_2 | 51 | GACTGAACATAACTGTAGGCTG | EGFR (−) | |
| | | | primer | |

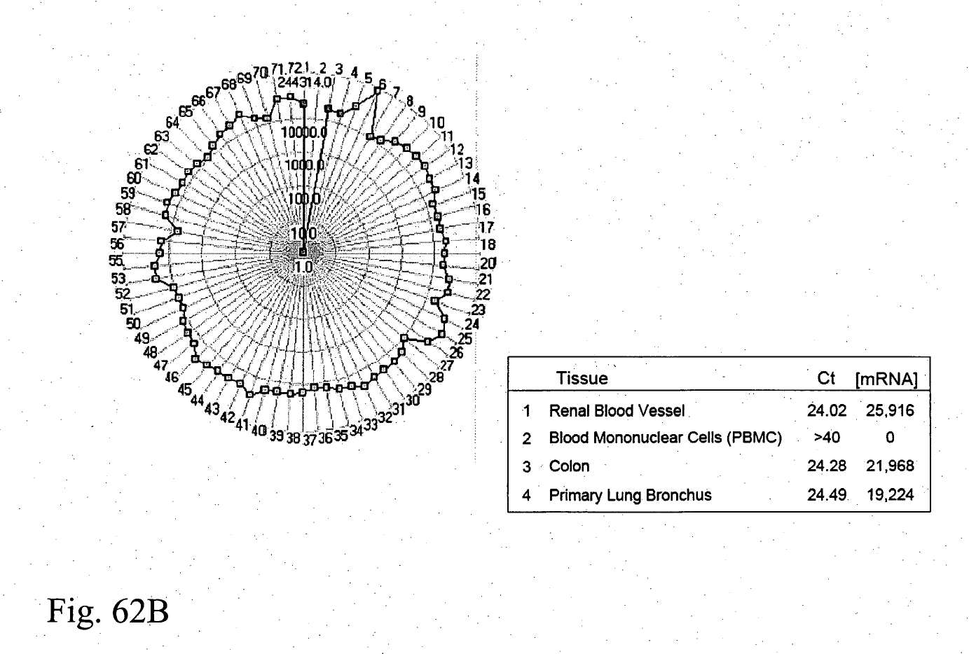

Total RNAs of approximately 9×10⁶ leukocytes isolated using a cell enrichment device of the invention (cutoff size 4 μm) and 5×10⁶ H1650 cells were isolated by using RNeasy mini kit (Qiagen). Two micrograms of total RNAs from leukocytes and H1650 cells were reverse transcribed to obtain first strand cDNAs using 100 pmol random hexamer (Roche) and 200 U Superscript II (Invitrogen) in a 20 μl reaction. The subsequent PCR was carried out using 0.5 μl of the first strand cDNA reaction and 10 pmol of forward and reverse primers in total 25 μl of mixture. The PCR was run for 40 cycles of 95° C. for 20 seconds, 56° C. for 20 seconds, and 70° C. for 30 seconds. The amplified products were separated on a 1% agarose gel. As shown in FIG. 62A, BCKDK was found to be expressed in both leukocytes and H1650 cells; CD45 was expressed only in leukocytes; and both EpCAM and EGFR were expressed only in H1650 cells. These results, which are fully consistent with the profile of EGFR expression shown in FIG. 62B, confirmed that EGFR is a particularly useful target for assaying mixtures of cells that include both leukocytes and cancer cells, because only the cancer cells will be expected to produce a signal.

### Example 6

### EGFR Assay with Low Quantities of Target RNA or High Quantities of Background RNA

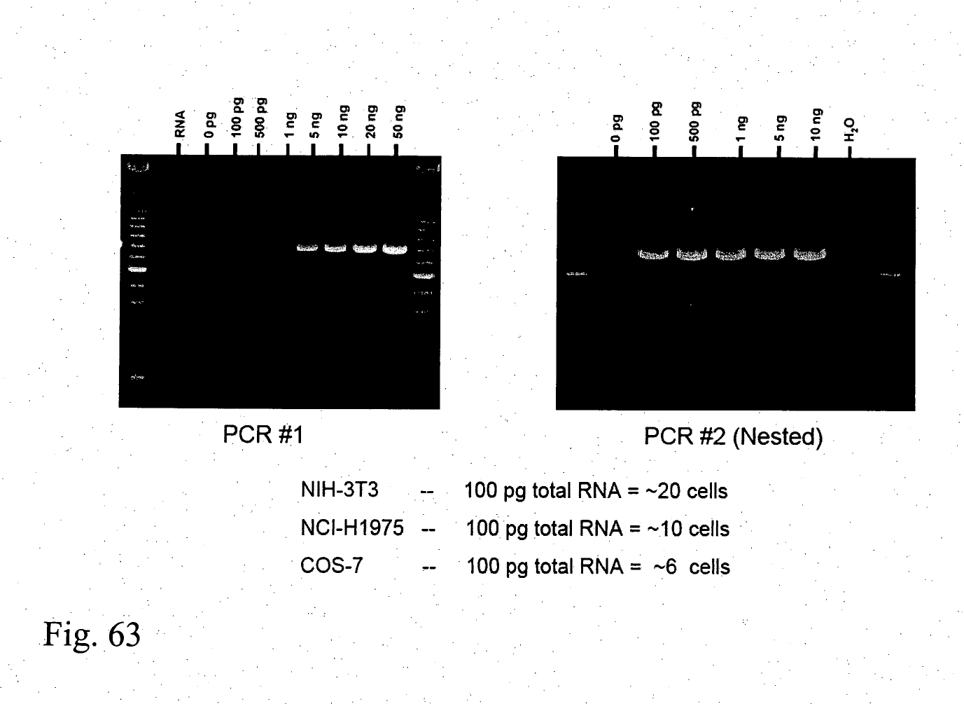

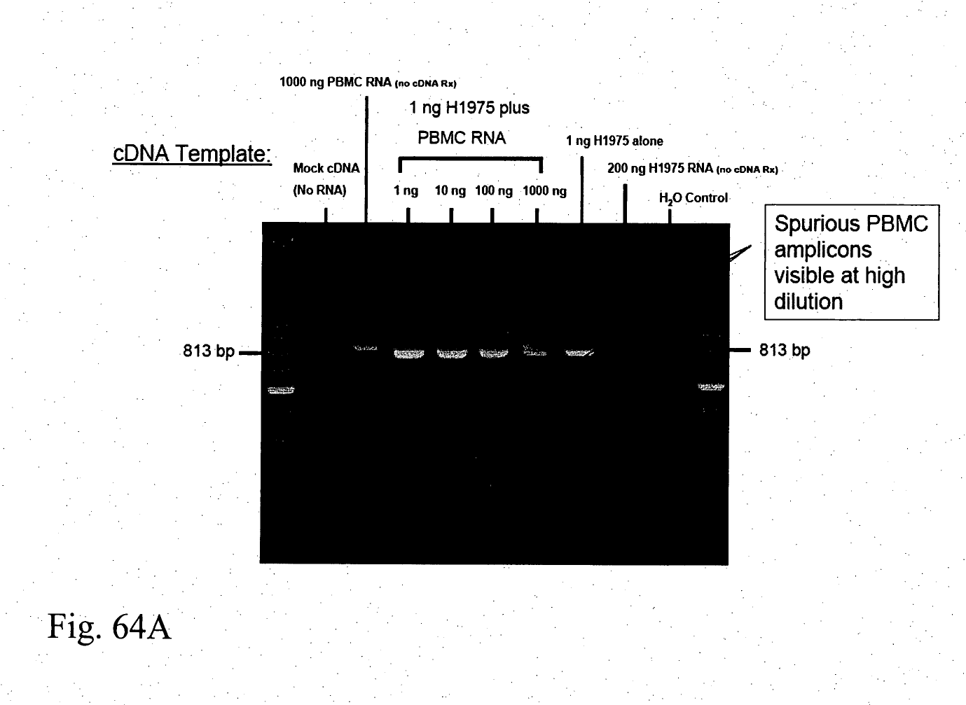

In order to determine the sensitivity of the assay described in Example 4, various quantities of input NSCLC cell line total RNA were tested, ranging from 100 pg to 50 ng. The results of the first and second EGFR PCR reactions (step 1d, Example 4) are shown in FIG. 63. The first PCR reaction was shown to be sufficiently sensitive to detect 1 ng of input RNA, while the second round increased the sensitivity to 100 pg or less of input RNA. This corresponds to 7-10 cells, demonstrating that even extremely dilute samples may generate detectable signals using this assay.

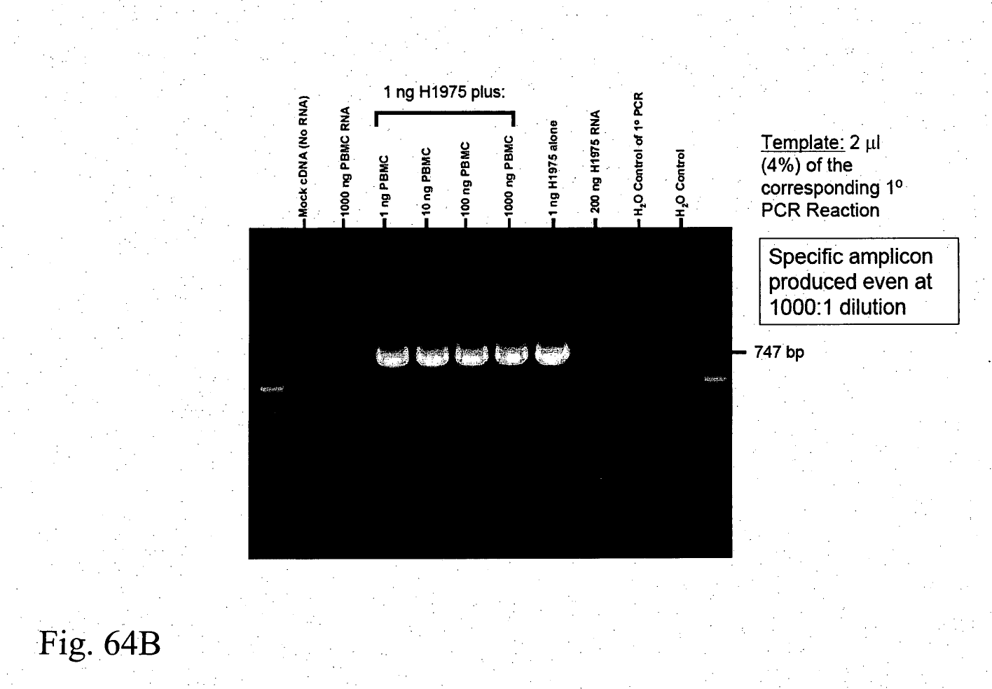

Next, samples containing 1 ng of NCI-H1975 RNA were mixed with varying quantities of peripheral blood mononuclear cell (PBMC) RNA ranging from 1 ng to 1 μg and used in PCR reactions as before. As shown in FIG. 64A, the first set of PCR reactions demonstrated that, while amplification occurred in all cases, spurious bands appeared at the highest contamination level. However, as shown in FIG. 64B, after the second, nested set of PCR reactions, the desired specific amplicon was produced without spurious bands even at the highest contamination level. Therefore, this example demonstrates that the EGFR PCR assays described herein are effective even when the target RNA occupies a tiny fraction of the total RNA in the sample being tested.

Table 7 lists the RNA yield in a variety of cells and shows that the yield per cell is widely variable, depending on the cell type. This information is useful in order to estimate the amount of target and background RNA in a sample based on cell counts. For example, 1 ng of NCL-H1975 RNA corresponds to approximately 100 cells, while 1 μg of PBMC RNA corresponds to approximately 10⁶ cells. Thus, the highest contamination level in the above-described experiment, 1,000: 1 of PBMC RNA to NCL-H1975 RNA, actually corresponds to a 10,000:1 ratio of PBMCs to NCL-H1975 cells. Thus, these data indicate that EGFR may be sequenced from as few as 100 CTCs contaminated by as many as 10⁶ leukocytes.

TABLE 7
RNA Yield versus Cell Type

| Cells | Count | RNA Yield | [RNA]/Cell |
|---|---|---|---|
| NCI-H1975 | 2 × 10⁶ | 26.9 μg | 13.5 pg |
| NCI-H1650 | 2 × 10⁶ | 26.1 μg | 13.0 pg |
| H358 | 2 × 10⁶ | 26.0 μg | 13.0 pg |
| HT29 | 2 × 10⁶ | 21.4 μg | 10.7 pg |
| MCF7 | 2 × 10⁶ | 25.4 μg | 12.7 pg |
| PBMC #1 | 19 × 10⁶ | 10.2 μg | 0.5 pg |
| PBMC #2 | 16.5 × 10⁶ | 18.4 μg | 1.1 pg |

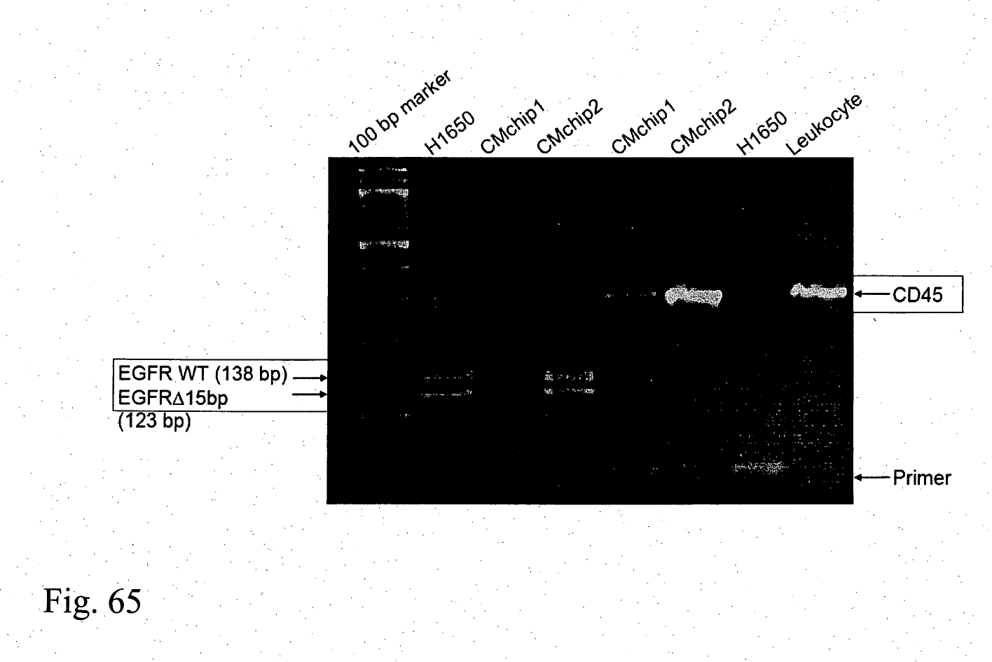

Next, whole blood spiked with 1,000 cells/ml of Cell Tracker (Invitrogen)-labeled H1650 cells was run through the capture module chip of FIG. 57C. To avoid inefficiency in RNA extraction from fixed samples, the captured H1650 cells were immediately counted after running and subsequently lysed for RNA extraction without formaldehyde fixation. Approximately 800 captured H1650 cells and >10,000 contaminated leukocytes were lysed on the chip with 0.5 ml of 4M guanidine thiocyanate solution. The lysate was extracted with 0.5 ml of phenol/chloroform and precipitated with 1 ml of ethanol in the presence of 10 μg of yeast tRNA as carrier. The precipitated RNAs were DNase I-treated for 30 minutes and then extracted with phenol/chloroform and precipitated with ethanol prior to first strand cDNA synthesis and subsequent PCR amplification. These steps were repeated with a second blood sample and a second chip. The cDNA synthesized from chip1 and chip2 RNAs along with H1650 and leukocyte cDNAs were PCR amplified using two sets of primers, CD45_{—}1 and CD45_{—}2 (Table 6) as well as EGFR_{—}5 (forward primer, 5′-GTTCGGCACGGTGTATAAGG-3′) (SEQ ID NO: 52) and EGFR_{—}6 (reverse primer, 5′-CTGGCCATCACGTAGGCTTC-3′) (SEQ ID NO: 53). EGFR_{—}5 and EGFR_{—}6 produce a 138 bp wild type amplified fragment and a 123 bp mutant amplified fragment in H1650 cells. The PCR products were separated on a 2.5% agarose gel. As shown in FIG. 65, EGFR wild type and mutant amplified fragments were readily detected, despite the high leukocyte background, demonstrating that the EGFR assay is robust and does not require a highly purified sample.

### Example 7

### Protocol for Processing a Blood Sample Through an Enrichment Module Coupled to a Capture Module

Using a sample of healthy blood spiked with tumor cells, a device of the invention containing an enrichment module coupled to a capture module was tested for the ability to enrich and capture tumor cells from blood.

To prepare the blood sample, a human non-small-cell lung cancer line, NCI-H1650 from ATCC) was stained with cell tracker orange (CMRA from Molecular Probes) and then spiked into fresh blood from a healthy patient (Research Blood Component). The spike level was 1,000 cells/ml. The spiked blood was diluted to a ratio of 2:1 (blood to buffer, 1% BSA in PBS). Both leukocytes and tumor cells were labeled with nuclear staining dye, Hoechst 33342; labeling the tumor cells with an additional stain, cell tracker orange, helped to distinguish tumor cells from leukocytes.

Next, the enrichment module manifold, chip, and tubing were set up, and the enrichment module chip was primed with degassed buffer. The spiked blood sample was run through the enrichment module at a pressure of 2.4 psi, and the flow rate of product was 6.91 ml/hr.

Prior to running the product through the capture module, the product was characterized. Taking into account the dilution factor in the product, the number of leukocytes per ml of equivalent whole blood was 7.02×10⁵. The removal efficiency of leukocytes was 90%. The yield of tumor cells was 89.5%, and the purity of the tumor cells was 0.14%.

The product from the enrichment module was then run through the capture module, which contained anti-EpCAM-coated obstacles. The tumor cells expressing epithelial cell adhesion molecule were captured on the obstacles. The flow rate was 2.12 ml/hr, and the running time was one hour. The device was then washed with buffer at a higher flow rate, 3 ml/hr, to remove the nonspecifically-bound cells. The yield was 74%. The purity was not determined.

The results of these experiments are summarized in Table 8.

TABLE 8

| | Yield of | Number of | Tumor | Yield of | Number of | Tumor | |
|---|---|---|---|---|---|---|---|
| | enrichment | leukocytes/ml | cell | capture | leukocytes/ml | cell | |
| | module | of whole | purity | module | of whole | purity | Combined |
| | (%) | blood | (%) | (%) | blood | (%) | yield (%) |
| Enrichment | 89 | 7.02 × 10⁵ | 0.14 | 74 (2.12 ml/hr) | Not measured | N/A | 66 |
| module | | | | | | | |
| (V1) - | | | | | | | |
| capture | | | | | | | |
| module | | | | | | | |

### Example 8

### Cell Capture Using Staggered Arrays

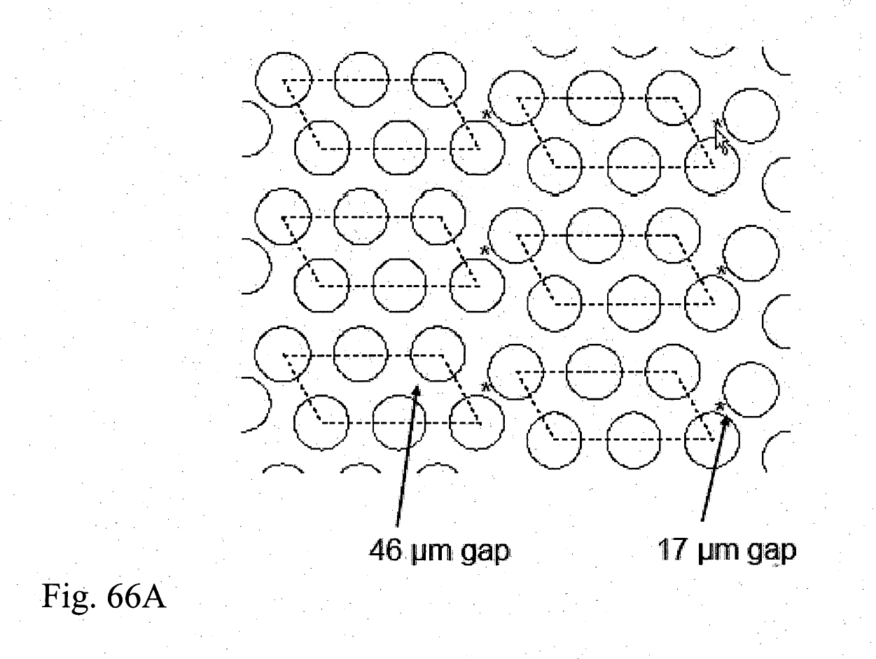

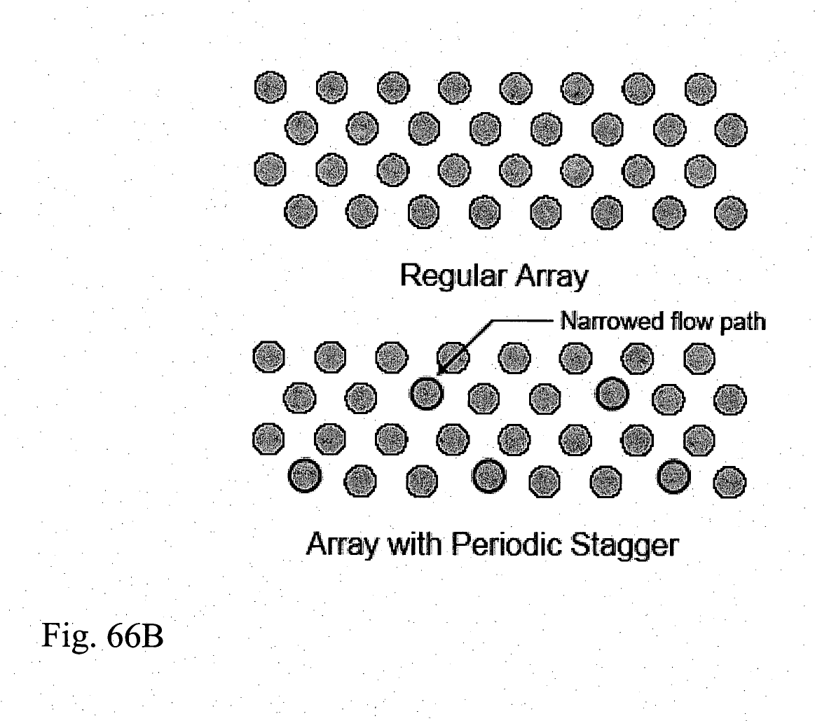

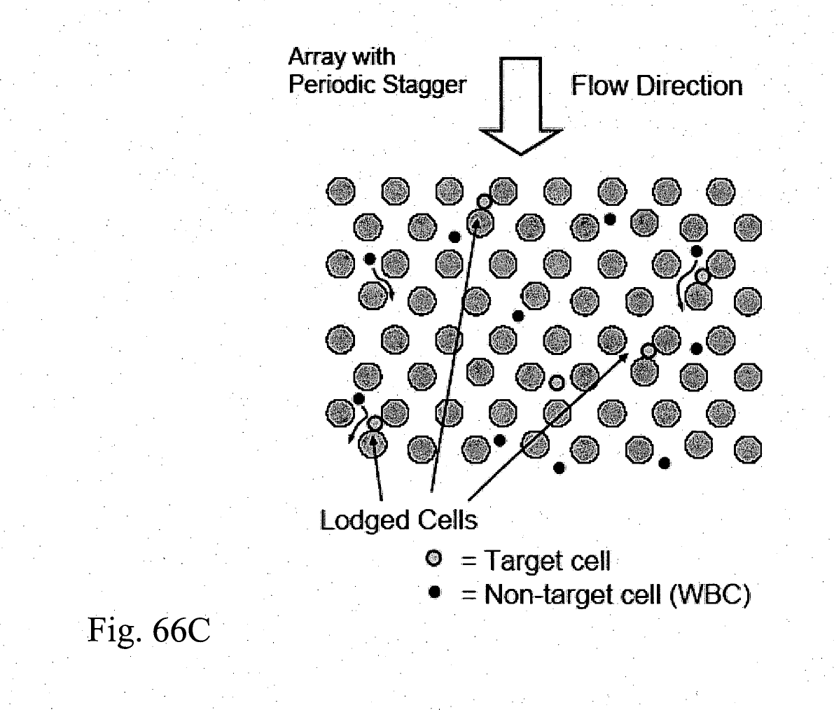

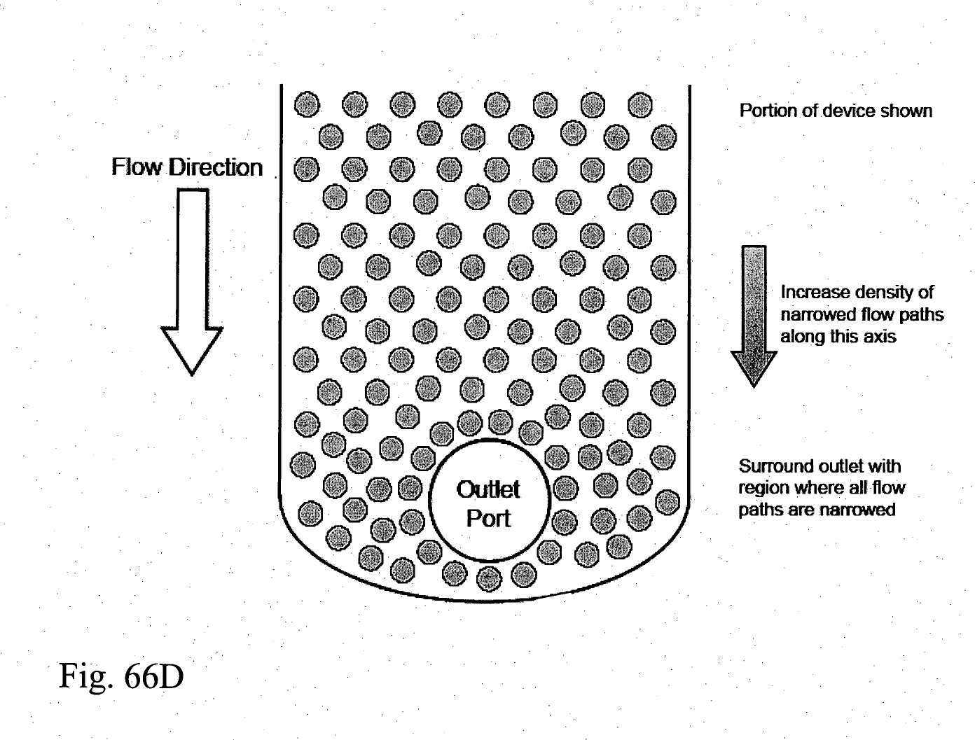

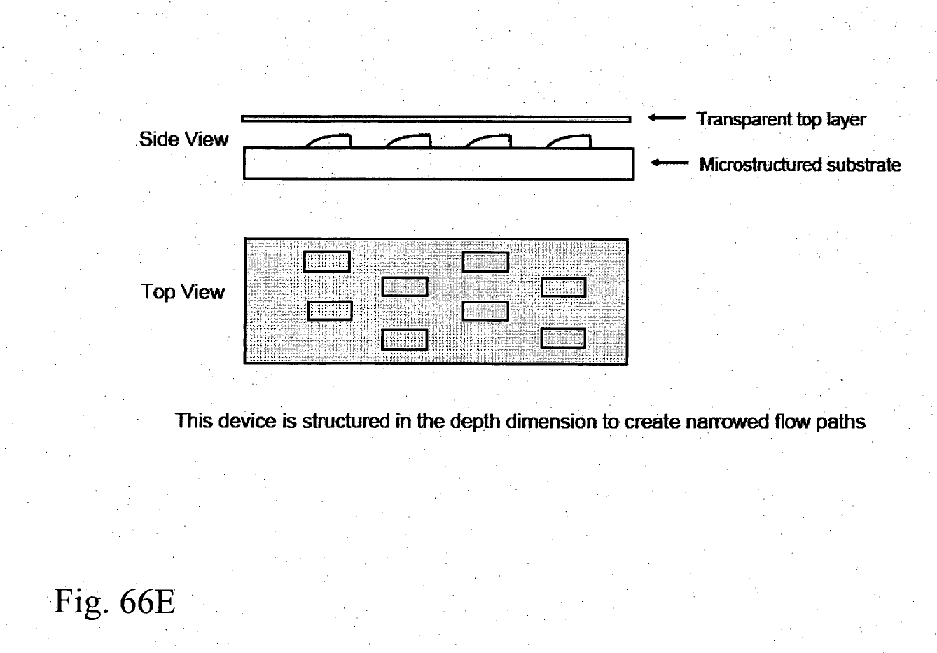

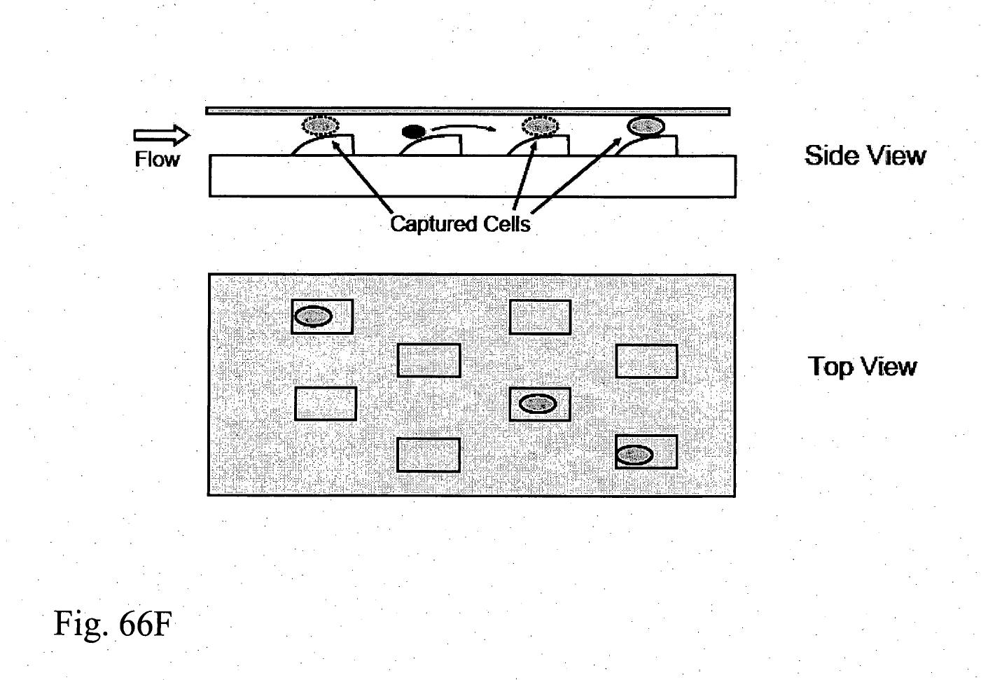

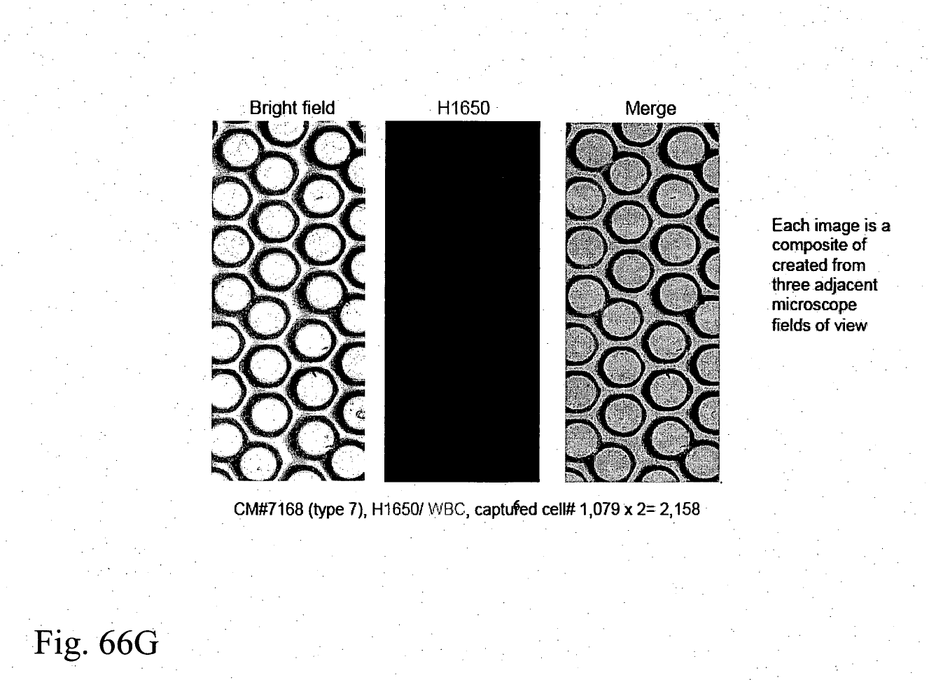

In one embodiment of the invention, CTCs or other cells larger than a chosen cutoff size may be captured using a device that includes obstacles arranged in an array of subarrays. The subarrays are arrayed over the field with a slight stagger, or uneven spacing, initially designed in order to introduce variation in the flow lines and encourage the interaction of cells with the obstacles. One effect of this arrangement is that each subarray gives rise to a region in which the flow path is narrowed, as shown in FIG. 66A. In the array shown in the figure, the regular gap between obstacles is 46 μm, while the narrowed gap is 17 μm. The array and subarrays may be varied in order to result in any desirable gap sizes, as well as any desired density of narrowed gaps in relation to regular gaps.

Such a staggered array is particularly useful for preferential capture of CTCs in a blood sample, since CTCs tend to be larger than most other blood cells. CTCs or other large cells may be captured within the array without the need for a functionalized surface containing antibodies or other binding moieties, since cell capture is based on array geometry. Fabrication of such a device is therefore simplified.

A staggered array of the invention is shown in FIG. 66B. Narrowed flow paths are dispersed regularly throughout the device, and these paths may be sized to capture cells of a given hydrodynamic size or larger, while allowing cells smaller than this cutoff size to flow through the array without being retained. If a large cell is lodged in a narrow flow path, thereby blocking it, smaller cells are still able to flow around via the unblocked larger flow paths, as shown in FIG. 66C. This design avoids the problem of clogging that may occur in a uniform array.

Desirably, the device is configured such that CTCs or other cells of interest are statistically likely to encounter and be trapped in the areas of narrowed gaps. Devices may be optimized for particular applications by varying the density of the restricted flow paths to alter the probability of capture of target cells.

In one configuration, a larger percentage of flow paths near the device outlet may be designed to be narrow (FIG. 66D), thereby allowing for capture of any large cells that were not captured elsewhere in the array. Unless all available narrow gaps are occupied by target cells, clogging is still avoided in this configuration.

Some devices of the invention have a relatively large depth dimension in order to accommodate high throughput of sample, whereas in other embodiments, the depth dimension is much smaller, with the result that captured cells are largely found in one focal plane and are easier to view under a microscope. In the device shown in FIG. 66E, the depth dimension is structured to create narrowed flow paths, resulting in capture of cells in a single focal plane (FIG. 66F). The captured cells are directly below the transparent window for simplified viewing. Fabrication of such devices may be achieved readily by a variety of means, e.g., injection molding or hot embossing of polymer substrates.

Once captured, cells may be released, e.g., by treatment with a hypotonic solution that causes the cells to shrink and be released from the device. Upon release and collection, cells may be returned to their original osmolarity and subjected to further analysis, e.g, molecular analysis. Alternatively, analysis may be conducted within the device without releasing the cells.

### Example 9

### Cell Capture of H1650 Cells Using Staggered Arrays

A capture module chip (FIG. 57C) was used to process a sample of H1650 lung cancer cells. Parameters of the capture module are as follows: the chip dimensions are 66.0×24.9 mm; the obstacle field dimensions are 51.3×18.9 mm; the obstacle diameter is 104 μm; the port dimensions are 2.83×2.83 mm on the front side and 1.66×1.66 mm on the back side; the substrate is silicon; and the etch depth is 100 μm. The H1650 lung cancer cells were spiked at 10,000 cells/ml into buffy coat and run at 1.6 ml/hour (FIG. 66G). An estimated 12,700 H1650 cells passed through the device. The device contained approximately 7,230 capture locations in the active area. The yield of H1650 cells following the experiment was 16%, indicating that a substantial portion of available capture locations was occupied by H1650 cells.

### Example 10

### Size Distribution of Cancer Cells

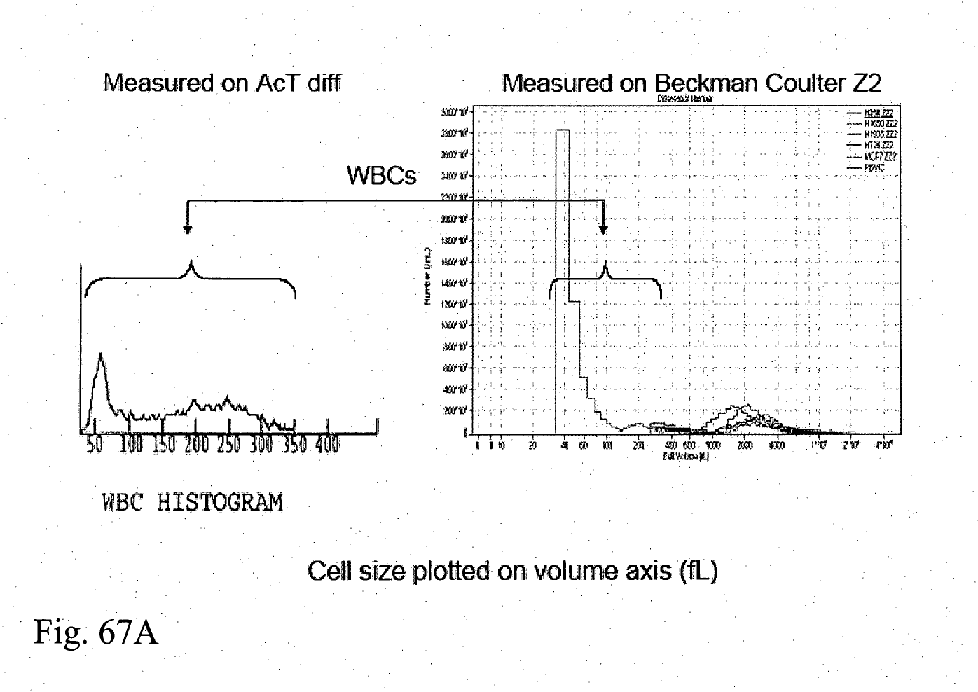

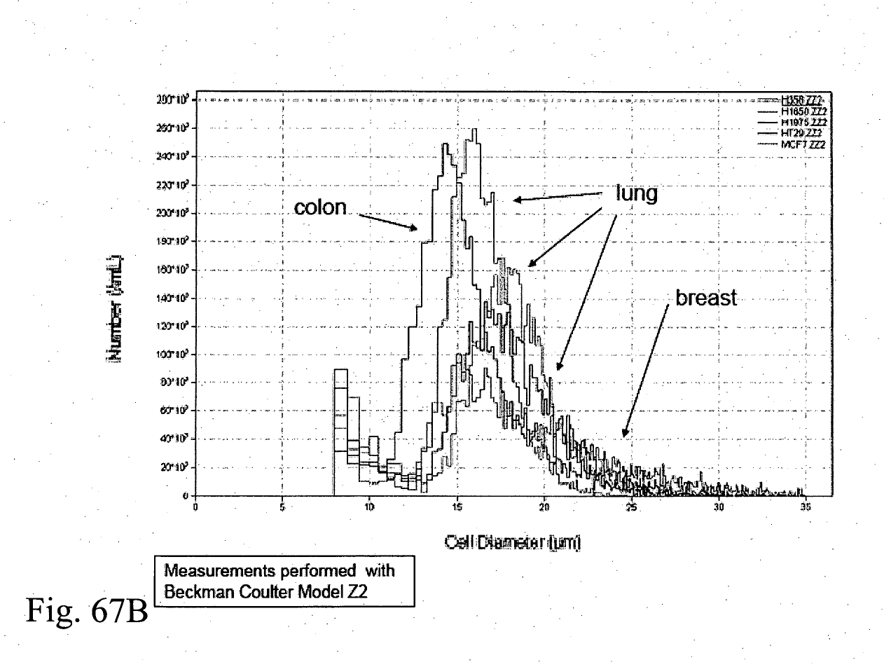

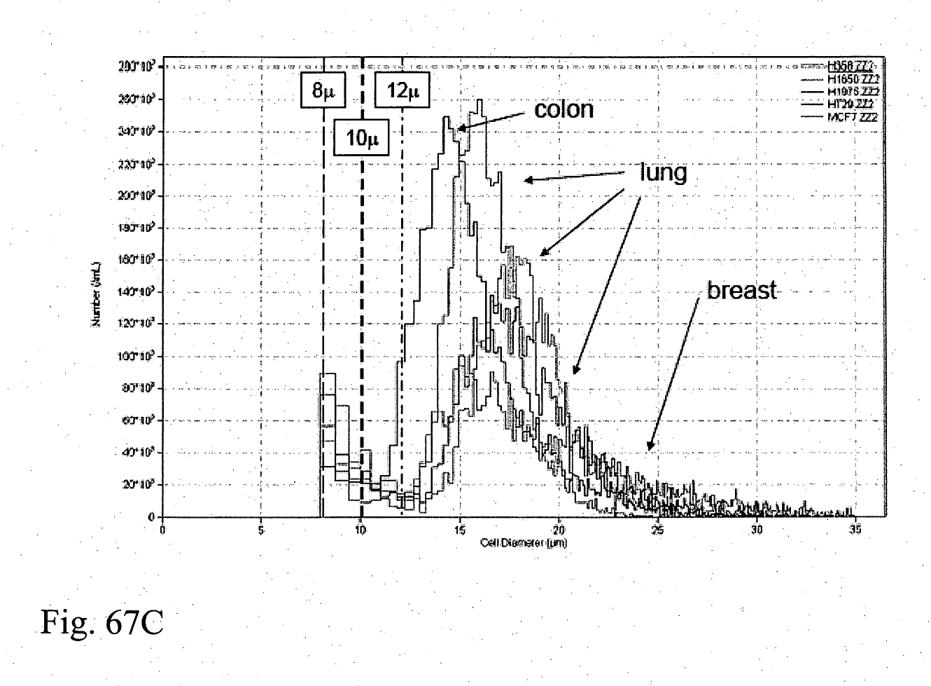

In order to determine the size distribution of cancer cells, several cancer cell lines were passed through a Beckman Coulter Model Z2 counting device (FIG. 67A). Cell lines that were tested in this experiment included H358, H1650, H1975, HT29, and MCF7 cells, which include colon, lung, and breast cancer cells. As FIG. 67A shows, each of these cell lines consists of cells that are larger than most white blood cells. The size distributions of each cancer cell line are similar to each other and are well-separated from the distribution of white blood cells shown. A closeup of the size distribution of the cancer cells (FIG. 67B) reveals a generally Gaussian distribution of cells in each case, with only a small minority of cells below 8, 10, or even 12 μm in size (FIG. 67C). These data offer strong support for the principle of enrichment of CTCs from other blood cells based on size.

### Example 11

### Capture Device Using a Microscope Slide

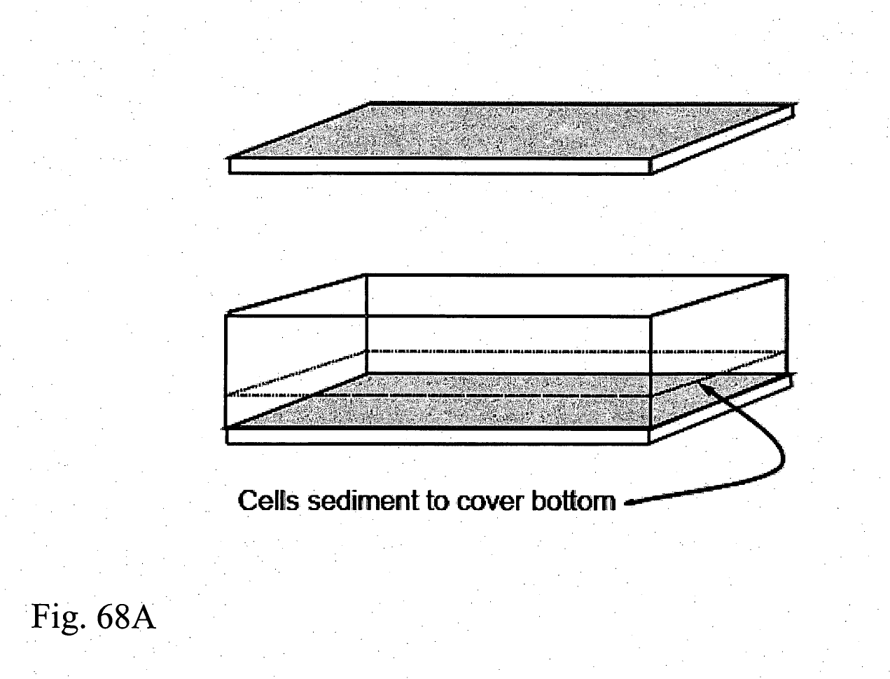

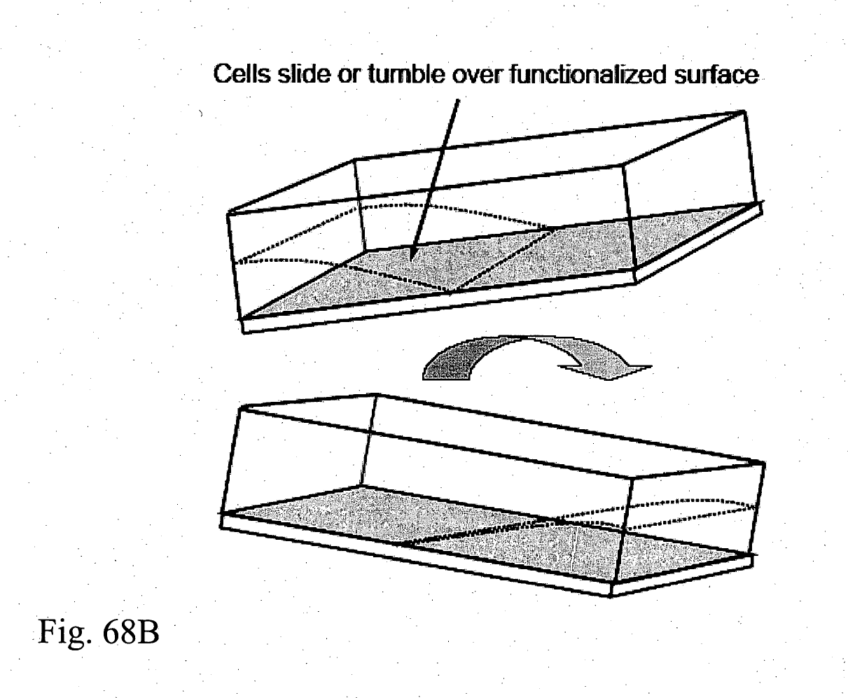

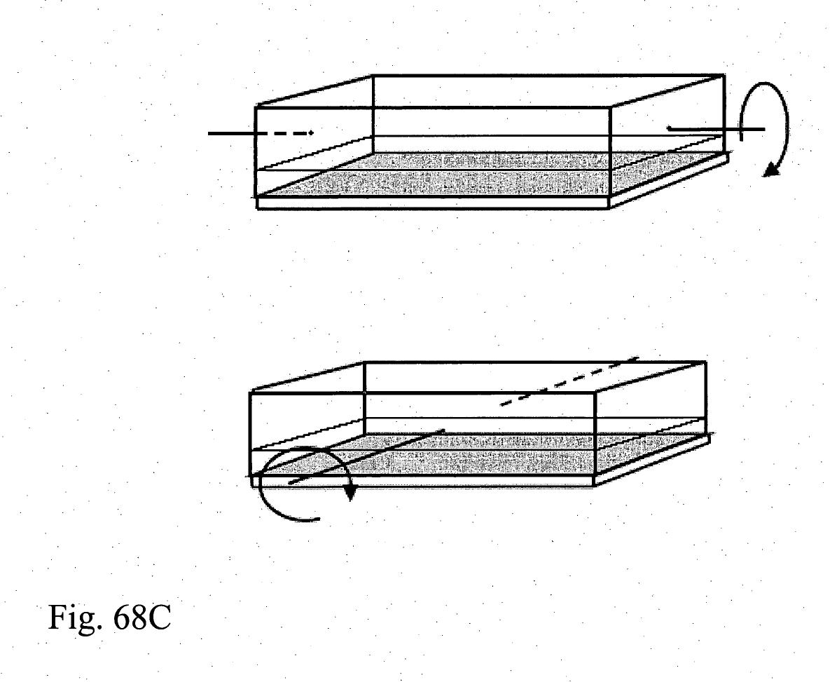

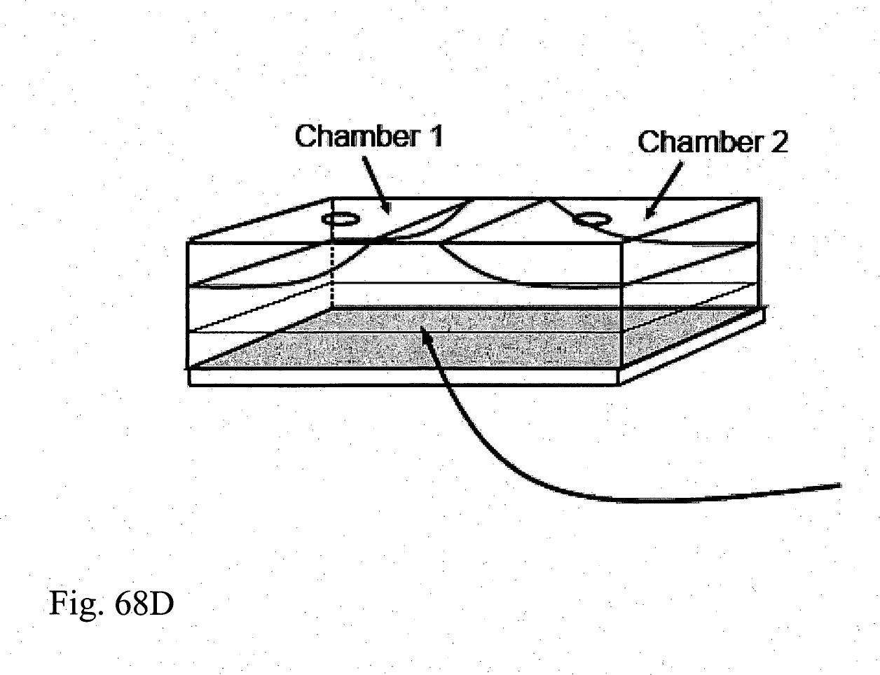

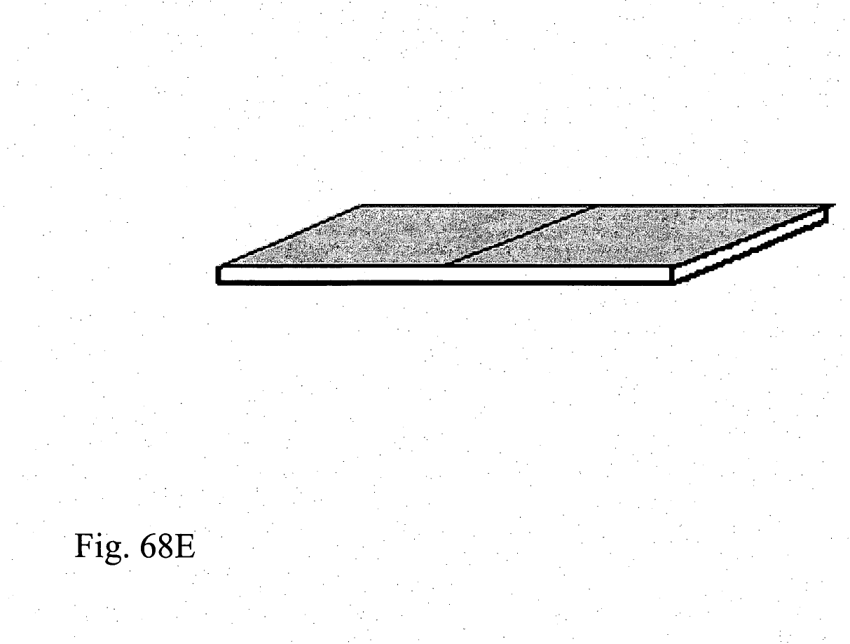

The invention encompasses a variety of cell capture devices and methods. In one embodiment, a capture device of the invention utilizes a functionalized surface, e.g., a glass microscope slide, as shown in FIG. 68A. The slide may be functionalized with an antibody or other capture moiety specific for the cell type of interest, e.g., CTCs, using standard chemistries. The device includes a sample fluid chamber, which may have, for example, a capacity of 10 ml or greater, with the functionalized slide on the bottom of the chamber. Any fluid, e.g., blood or a blood fraction, may be placed within the chamber for processing.

Cells within the fluid sample sediment to the bottom of the chamber via gravity, or optionally centrifugation (see Example 12), or application of other forces, and are bound by the functionalized surface. In order to keep the remaining cells tumbling, the chamber may be rocked (FIG. 68B) or rotated (FIG. 68C). Subsequently, the chamber may be washed and removed, and the slide is then available for staining, visualization, and/or other subsequent analysis.

Several advantages of such a device and method are evident. For example, the flat capture surface allows for easy visualization of captured cells. Furthermore, the uniform cell capture on the flat surface simplifies cell quantification. In addition, the residence time for cells contacting the surface is long in comparison to other methods, improving capture efficiency and allowing for the total duration of the experiment to be shortened. This duration may also be shortened in view of the fact that there is no limiting flow rate. Because the cells are not flowing through a device, they are also not subjected to flow-induced shear.

Other advantages include the fact that, in the configuration described here, surface area is generally not a limiting factor in the capture of rare cells. Furthermore, it is particularly straightforward to analyze captured cells using a light microscope or other visualization techniques, allowing for the analysis of morphology, organelle characteristics, or other cellular characteristics.

The capture device may be coupled to other devices for processing cellular samples or other fluid samples, and it is compatible with microcapture technologies.

In one variation, shown in FIG. 68D, two additional fluid chambers are present in the device. The fluid chambers, which may be filled with air, are alternately filled and emptied in order to cause fluid motion inside the main chamber of the device. The air chambers have a flexible wall separating them, and may be filled and emptied using any mechanism. The device mobilizes the cellular sample or other fluid sample, keeping sedimented cells tumbling and preventing the blockage of capture sites on the functionalized surface.

The capture surface of any of the above devices may be microstructured, e.g., with low relief, including micro-posts, micro-fins, and/or micro-corrugation. The functionalized surface may be, e.g., a microfabricated silicon chip surface or a plastic surface. This approach provides, for example, multiple, spatially patterned capture functionalities on the surface for differential capture, quantification, and/or targeting of multiple cell populations (FIG. 68E).

### Example 12

### Centrifugal Capture Device Using a Microscope Slide

Prior to using a capture device of the invention, it is advantageous to perform microfluidics-based cell enrichment with a cell enrichment device of the invention. For example, by applying a first enrichment step to a blood sample, most erythrocytes, leukocytes, and platelets are removed. In one set of experiments, when blood samples were processed using cell enrichment devices of the invention having a cutoff of 8 μm, 10 μm, and 12 μm, erythrocytes and platelets were removed completely in each case, and the leukocyte concentration was reduced to 1.25×10⁵ cells/ml, 2,900 cells/ml, and 111 cells/ml, respectively. Thus, a large portion of the contaminating cells in a blood sample or other cellular sample may be removed prior to a capture step, helping to avoid nonspecific sedimentation on a functionalized surface. However, the resulting enriched sample may be highly diluted, thereby increasing the processing time necessary to capture cells of interest, e.g., CTCs.

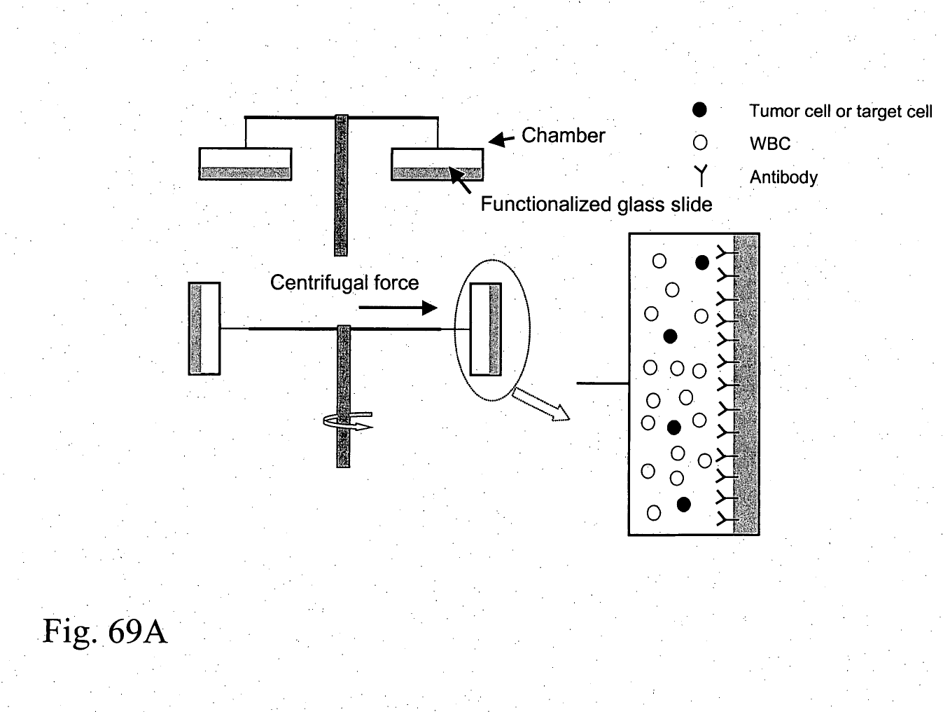

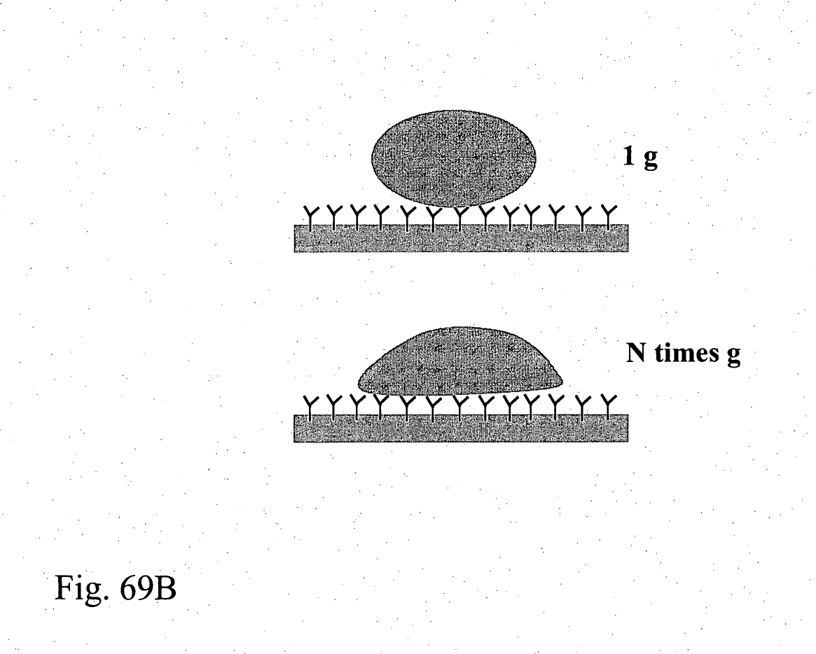

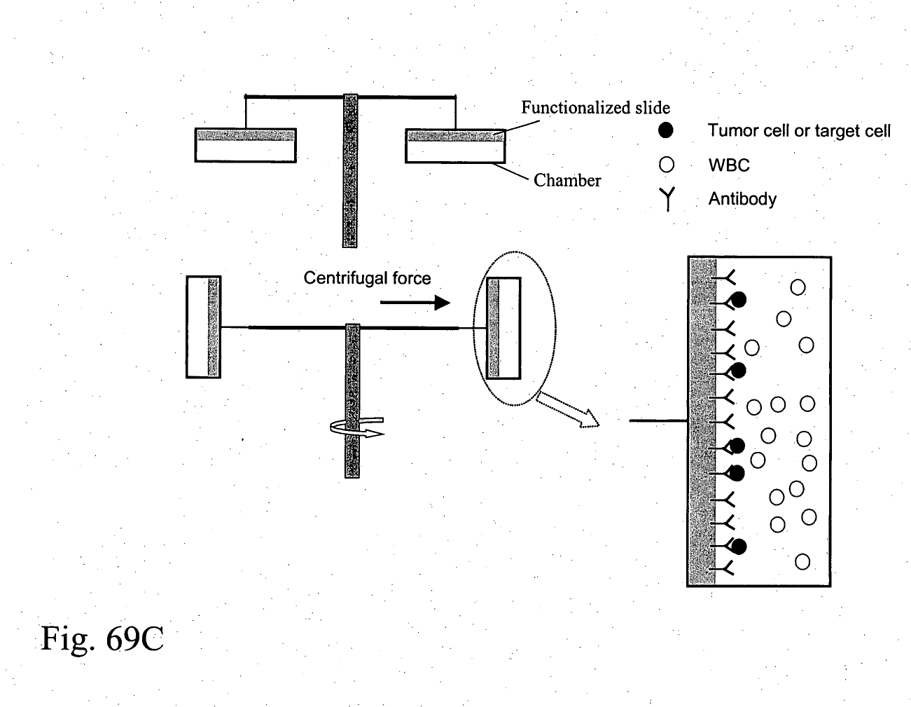

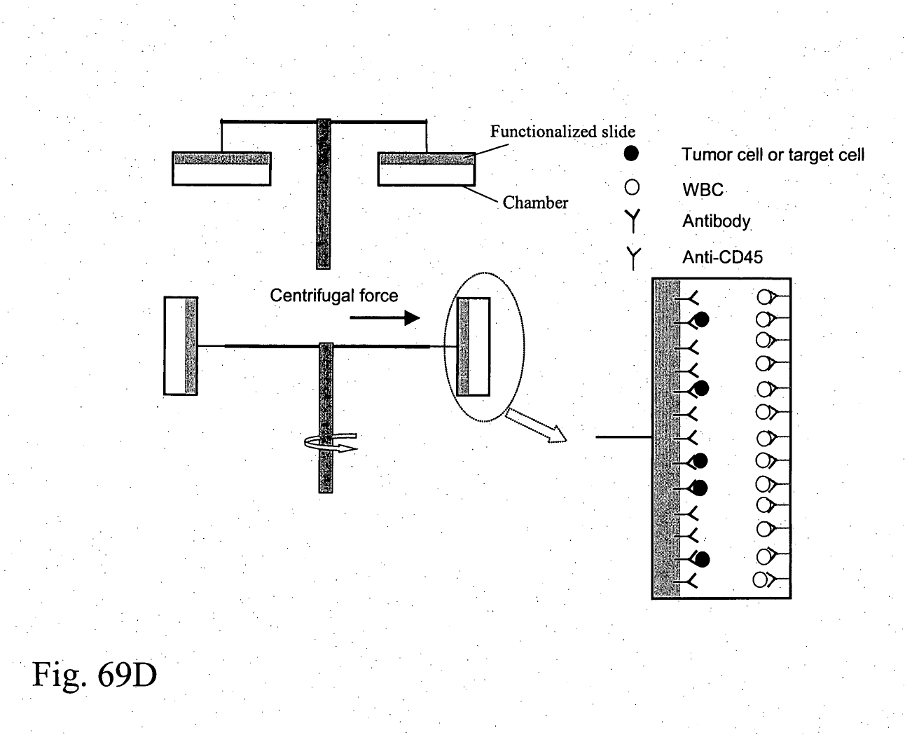

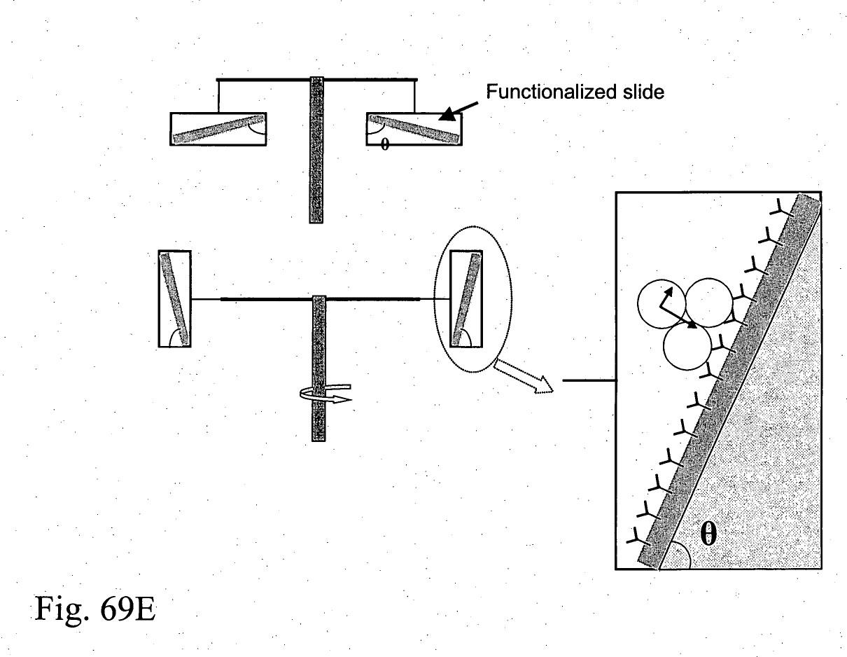

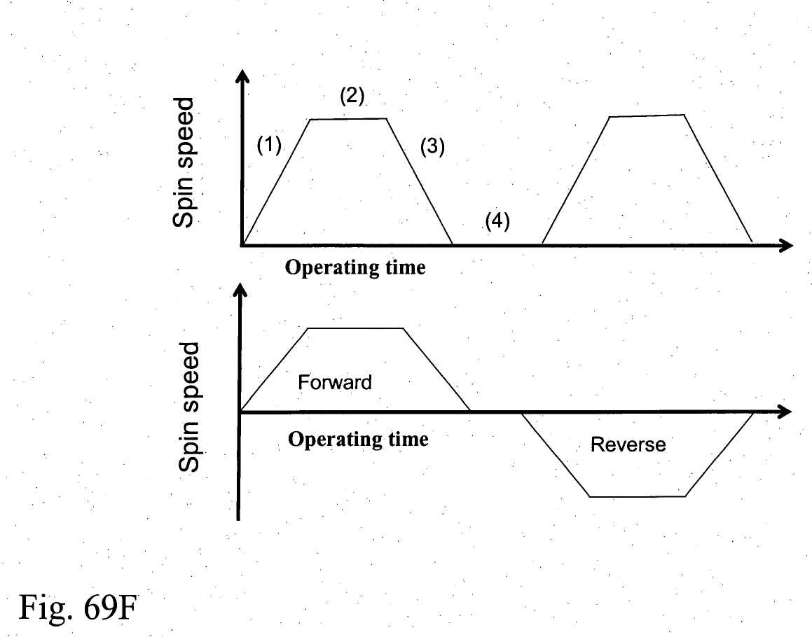

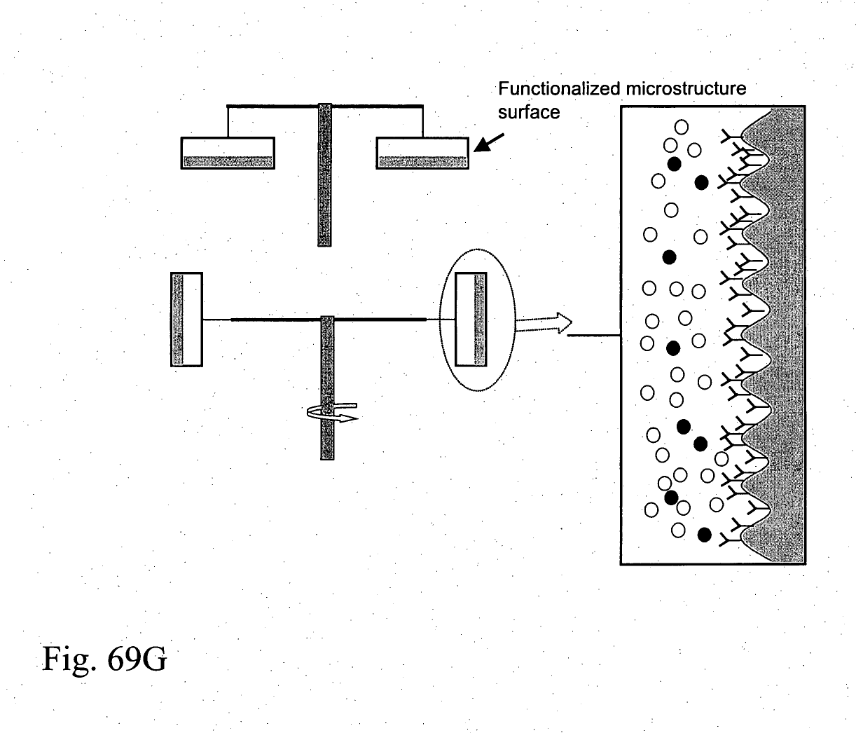

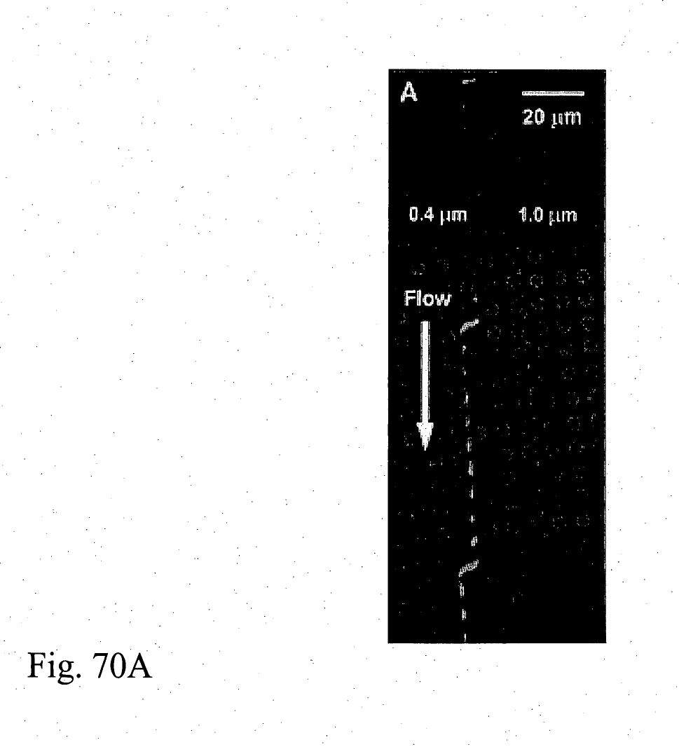

To decrease the time required to process a sample, the device described in Example 11 may be used in combination with a centrifuge (FIG. 69A). In this method, cells of interest, e.g., CTCs, are flattened against the functionalized slide (FIG. 69B) when the sample is exposed to a high centrifugal field of N×g, where, for example, N is a large number, e.g., 1,000 or greater. This centrifugal method substantially increases the contact location and area between CTCs and binding moieties, e.g., antibodies.

Cell sedimentation velocity may be estimated by the equation:

$$u = \frac{{ad}_{cell}^{2} ( {ρ}_{cell} - {ρ}_{plasma} )}{18 {μ}_{plasma}}$$

where u represents velocity, d represents cell diameter, ρ represents density, μ represents viscosity, and a represents acceleration, i.e., gravitational or centrifugal field. The parameter a may be expressed as N×g, where N equals 1 in the case of gravity, and N generally equals a large number, e.g., 1,000 or greater, in the case of centrifugation. When N equals 1, i.e., in the presence of gravity alone, it takes approximately one hour for a 14 μm diameter cell to settle in a 2 cm high liquid level chamber; however, with a centrifugal field of N×g, sedimentation time is reduced by a factor of N, thereby significantly reducing the time required to perform the experiment.

Following capture of CTCs, leukocytes or other contaminating cells that are bound nonspecifically to the functionalized surface may be removed by inverting the chamber and subjecting it once again to a high centrifugal force (FIG. 69C). This step greatly reduces the number of contaminating cells that remain attached to the functionalized surface. In one embodiment, antibodies specific for contaminating cells such as leukocytes may be coupled to a functionalized surface opposite the surface that is used to capture the cells of interest (FIG. 69D), thereby capturing the contaminating cells and further minimizing contamination of the captured cells of interest. In another variation, the functionalized surface used to capture cells of interest may be inclined at an angle, resulting in a centrifugal force component that drives cell rolling along the planar surface, in addition to the perpendicular component of the centrifugal force (FIG. 69E). The component of the centrifugal force that drives cell rolling helps to spread clusters of cells and increases the efficiency of cell capture.

The applied centrifugal field may be optimized in a number of ways (FIG. 69F). For example, each period of centrifugation may be modified, including the “spin up” phase (period between starting centrifugation and attaining the desired rotational speed), “spin time” (period of centrifugation at desired rotational speed), “spin down” (period between beginning to slow centrifugation and coming to a stop), and “rest time” (period between spins). In each case, the duration, rotational speed, and/or rotational acceleration may be optimized to suit the application. This includes spinning the chamber in both the forward and reverse directions, as described above.

To improve capture efficiency, the functionalized surface may be micro-structured (FIG. 69G), as in Example 11.

### Example 13

### Capture Device

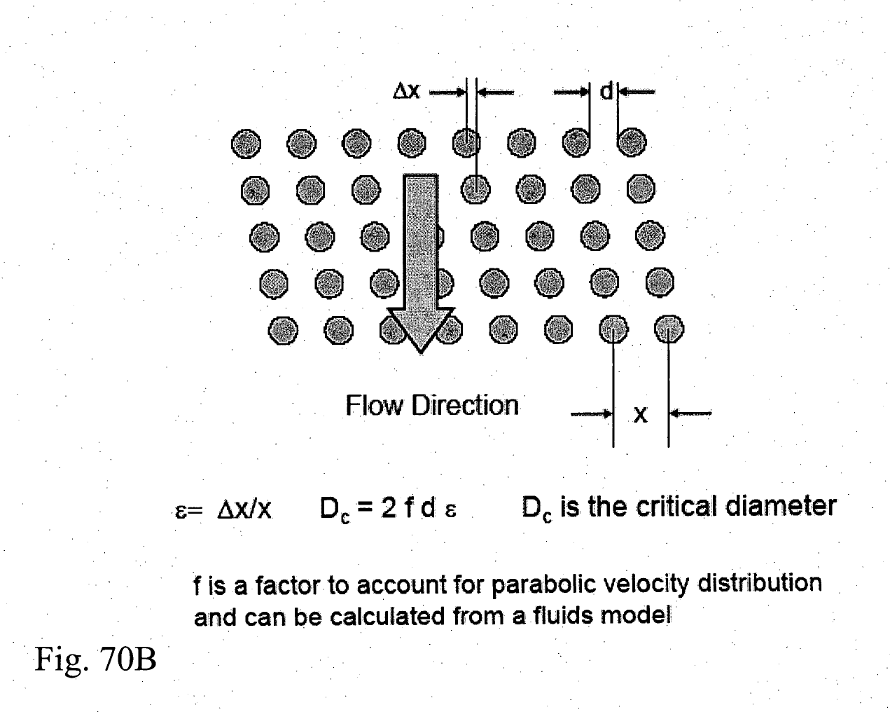

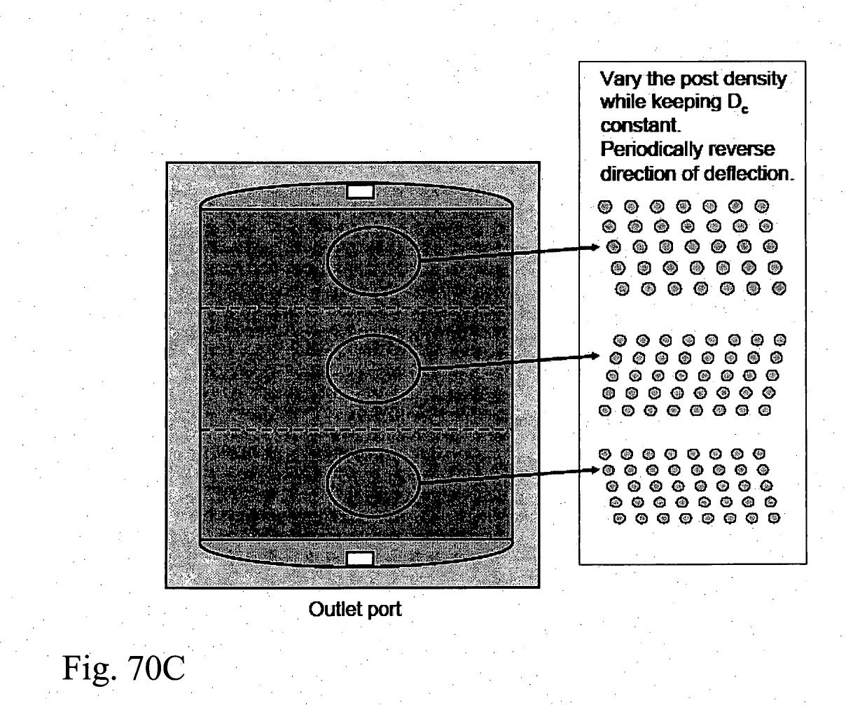

In the enrichment devices of the invention that include obstacles (FIG. 70A, and described above), large cells generally have numerous interactions with the obstacles, while small cells are able to flow through the device with minimal contact with the obstacles. A capture device that includes antibodies or other binding moieties attached to the surfaces of arrayed obstacles may be designed using similar principles, and combines both size and affinity selectivity.

In a regular array of obstacles, the critical diameter depends on a number of parameters, including the gap size and the distance between obstacles (obstacle offset), as shown in FIG. 70B. As described above, cells that are larger than the critical diameter are deflected, while cells that are smaller than this parameter move in the average flow direction. Thus, based on the size of the cell type of interest, e.g., a particular type of CTC, the critical diameter may be optimized. This may be achieved, for example, by selecting an appropriate gap size and offset. The optimized device may provide efficient capture with very low contamination.

In one instance, the obstacle density may be varied throughout the device. For example, obstacles may be arrayed at a lower density near the sample inlet of the device, or order to prevent clogging, while the density may be increased near the device outlet, in order to maximize capture.

It is possible to vary the arrangement of obstacles while keeping the critical size constant. Thus, devices of the invention may include variable obstacle arrays in which the direction of deflection, the gap size, and/or the distance between obstacles is varied throughout the device, in order to increase flow rate, decrease clogging, or achieve other design goals (FIG. 70C).

In some devices, both target cells, e.g., CTCs, and contaminating cells, e.g., leukocytes, bind to the floor of the device. For example, this may occur in devices that include a functionalized silicon substrate containing obstacles, as all exposed surfaces of the silicon substrate are typically functionalized with antibody or other binding moiety. Thus, capture devices may be operated in an inverted orientation, such that any cells that sediment come into contact with a non-functionalized surface and do not bind. This may result in reduced clogging and may generally improve device performance.

The capture device described in this example, or other capture devices of the invention, may also include nonfunctionalized areas that may be used for enrichment or other purposes.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A device for processing a cellular sample, said device comprising a channel comprising a first array of obstacles that form a network of gaps, wherein said obstacles are configured to cause one or more first cells to preferentially make contact with said obstacles, and wherein at least some of said obstacles comprise one or more binding moieties that selectively bind said first cells.

2. The device of claim 1, wherein said first cells comprise epithelial cells, cancer cells, bone marrow cells, fetal cells, progenitor cells, stem cells, foam cells, mesenchymal cells, immune system cells, endothelial cells, endometrial cells, connective tissue cells, trophoblasts, bacteria, fungi, or pathogens.

3. The device of claim 2, wherein said first cells comprise epithelial cells or cancer cells.

4. The device of claim 1, wherein said cellular sample comprises blood, sweat, tears, ear flow, sputum, lymph, bone marrow suspension, urine, saliva, semen, vaginal flow, cerebrospinal fluid, brain fluid, ascites, milk, secretions of the respiratory, intestinal or genitourinary tract, amniotic fluid, or a water sample.

5. The device of claim 1, wherein said array of obstacles is a two-dimensional array.

6. The device of claim 1, wherein said array of obstacles comprises a plurality of rows, each successive row being offset by less than half of the period of the previous row.

7. The device of claim 1, wherein said array of obstacles comprises a staggered two-dimensional array of obstacles.

8. The device of claim 7, said device further comprising a second staggered two-dimensional array of obstacles that form a network of gaps, wherein said first array and said second array are oriented in different directions.

9. The device of claim 1, said device further comprising a second array of obstacles that form a network of gaps, wherein said first array is situated upstream of said second array, and wherein said second array has a higher density than said first array.

10. The device of claim 9, said device further comprising one or more additional arrays of obstacles that form a network of gaps, wherein each additional array has an equal or higher density than any array upstream of said additional array.

11. The device of claim 1, wherein said gaps are at least 40 microns.

12. The device of claim 1, wherein said binding moieties specifically bind a cell surface cancer marker.

13. The device of claim 12, wherein said cell surface cancer marker is selected from Table 1.

14. The device of claim 12, wherein said cell surface cancer marker is selected from the group consisting of EpCAM, E-Cadherin, Mucin-1, Cytokeratin 8, EGFR, and leukocyte associated receptor (LAR).

15. The device of claim 1, wherein said binding moieties comprise a polypeptide.

16. The device of claim 15, wherein said polypeptide comprises an antibody.

17. The device of claim 16, wherein said antibody specifically binds one or more epithelial cells, cancer cells, bone marrow cells, fetal cells, progenitor cells, stem cells, foam cells, mesenchymal cells, immune system cells, endothelial cells, endometrial cells, connective tissue cells, trophoblasts, bacteria, fungi, or pathogens.

18. The device of claim 1, wherein said device comprises an inlet for delivery of said cellular sample.

19. The device of claim 18, wherein said device is configured for delivery of a second fluid medium through said inlet, said fluid medium comprising a buffer, a lysis reagent, a nucleic acid amplification reagent, an osmolarity regulating reagent, a labeling reagent, a preservative, or a fixing reagent.

20. The device of claim 1, wherein said device further comprises a programmable heating unit.

21. A method of detecting cancer cells in a cellular sample, said method comprising the steps of:
a) introducing said cellular sample into a device comprising a channel comprising a first array of obstacles that form a network of gaps, wherein said obstacles are configured to cause one or more first cells to preferentially make contact with said obstacles, and wherein at least some of said obstacles comprise one or more binding moieties that selectively bind said first cells; and
b) detecting the presence or absence of cancer cells in said device.

22. A method for diagnosing whether a subject has cancer, said method comprising the steps of:
a) introducing a cellular sample from said subject comprising one or more cancer cells into a device comprising a channel comprising a first array of obstacles that form a network of gaps, wherein said obstacles are configured to cause one or more first cells to preferentially make contact with said obstacles, and wherein at least some of said obstacles comprise one or more binding moieties that selectively bind said first cells;
b) detecting the presence or absence of said cancer cells in said device; and
c) diagnosing the presence or absence of cancer in said subject based on the results of step b).

23. The device of claim 1, wherein fluid flows through said gaps such that said fluid is divided unequally into a major flux and a minor flux.


SEQUENCE LISTING (53 sequences)

SEQ ID NO: 1 - DNA, 17 bp, artificial sequence
  synthetic
ttgctgctggtggtggc

SEQ ID NO: 2 - DNA, 20 bp, artificial sequence
  synthetic
cagggattccgtcatatggc

SEQ ID NO: 3 - DNA, 18 bp, artificial sequence
  synthetic
gatcggcctcttcatgcg

SEQ ID NO: 4 - DNA, 22 bp, artificial sequence
  synthetic
gatccaaaggtcatcaactccc

SEQ ID NO: 5 - DNA, 18 bp, artificial sequence
  synthetic
gctgtccaacgaatgggc

SEQ ID NO: 6 - DNA, 20 bp, artificial sequence
  synthetic
ggcgttctcctttctccagg

SEQ ID NO: 7 - DNA, 19 bp, artificial sequence
  synthetic
atgcactgggccaggtctt

SEQ ID NO: 8 - DNA, 21 bp, artificial sequence
  synthetic
cgatggtacatatgggtggct

SEQ ID NO: 9 - DNA, 19 bp, artificial sequence
  synthetic
aggctgtccaacgaatggg

SEQ ID NO: 10 - DNA, 19 bp, artificial sequence
  synthetic
ctgagggaggcgttctcct

SEQ ID NO: 11 - DNA, 22 bp, artificial sequence
  synthetic
tcagagcctgtgtttctaccaa

SEQ ID NO: 12 - DNA, 22 bp, artificial sequence
  synthetic
tggtctcacaggaccactgatt

SEQ ID NO: 13 - DNA, 20 bp, artificial sequence
  synthetic
tccaaatgagctggcaagtg

SEQ ID NO: 14 - DNA, 23 bp, artificial sequence
  synthetic
tcccaaacactcagtgaaacaaa

SEQ ID NO: 15 - DNA, 24 bp, artificial sequence
  synthetic
aaataatcagtgtgattcgtggag

SEQ ID NO: 16 - DNA, 22 bp, artificial sequence
  synthetic
gaggccagtgctgtctctaagg

SEQ ID NO: 17 - DNA, 20 bp, artificial sequence
  synthetic
gtgcatcgctggtaacatcc

SEQ ID NO: 18 - DNA, 20 bp, artificial sequence
  synthetic
tgtggagatgagcagggtct

SEQ ID NO: 19 - DNA, 21 bp, artificial sequence
  synthetic
acttcacagccctgcgtaaac

SEQ ID NO: 20 - DNA, 21 bp, artificial sequence
  synthetic
atgggacaggcactgatttgt

SEQ ID NO: 21 - DNA, 20 bp, artificial sequence
  synthetic
atcgcattcatgcgtcttca

SEQ ID NO: 22 - DNA, 20 bp, artificial sequence
  synthetic
atccccatggcaaactcttg

SEQ ID NO: 23 - DNA, 22 bp, artificial sequence
  synthetic
gcagcgggttacatcttctttc

SEQ ID NO: 24 - DNA, 22 bp, artificial sequence
  synthetic
cagctctggctcacactaccag

SEQ ID NO: 25 - DNA, 22 bp, artificial sequence
  synthetic
gcagcgggttacatcttctttc

SEQ ID NO: 26 - DNA, 19 bp, artificial sequence
  synthetic
catcctcccctgcatgtgt

SEQ ID NO: 27 - DNA, 20 bp, artificial sequence
  synthetic
ccgcagcatgtcaagatcac

SEQ ID NO: 28 - DNA, 25 bp, artificial sequence
  synthetic
tccttctgcatggtattctttctct

SEQ ID NO: 29 - DNA, 14 bp, artificial sequence
  synthetic
tttgggctggccaa

SEQ ID NO: 30 - DNA, 14 bp, artificial sequence
  synthetic
ttttgggcgggcca

SEQ ID NO: 31 - DNA, 17 bp, artificial sequence
  synthetic
atggccagcgtggacaa

SEQ ID NO: 32 - DNA, 23 bp, artificial sequence
  synthetic
agcaggtactgggagccaatatt

SEQ ID NO: 33 - DNA, 16 bp, artificial sequence
  synthetic
atgagctgcgtgatga

SEQ ID NO: 34 - DNA, 16 bp, artificial sequence
  synthetic
atgagctgcatgatga

SEQ ID NO: 35 - DNA, 22 bp, artificial sequence
  synthetic
gcctcttacacccagtggagaa

SEQ ID NO: 36 - DNA, 22 bp, artificial sequence
  synthetic
ttctgggatccagagtccctta

SEQ ID NO: 37 - DNA, 14 bp, artificial sequence
  synthetic
accggagcccagca

SEQ ID NO: 38 - DNA, 14 bp, artificial sequence
  synthetic
accggagctcagca

SEQ ID NO: 39 - DNA, 14 bp, artificial sequence
  synthetic
accggagcacagca

SEQ ID NO: 40 - DNA, 21 bp, artificial sequence
  synthetic
tcgcaaagggcatgaactact

SEQ ID NO: 41 - DNA, 21 bp, artificial sequence
  synthetic
atcttgacatgctgcggtgtt

SEQ ID NO: 42 - DNA, 14 bp, artificial sequence
  synthetic
ttggtgcaccgcga

SEQ ID NO: 43 - DNA, 14 bp, artificial sequence
  synthetic
tggtgctccgcgac

SEQ ID NO: 44 - DNA, 19 bp, artificial sequence
  synthetic
agtcaggacccatgcacgg

SEQ ID NO: 45 - DNA, 20 bp, artificial sequence
  synthetic
acccaagatgcagcagtgtg

SEQ ID NO: 46 - DNA, 21 bp, artificial sequence
  synthetic
gatgtcctccttgttctactc

SEQ ID NO: 47 - DNA, 22 bp, artificial sequence
  synthetic
tacagggaataatcgagcatgc

SEQ ID NO: 48 - DNA, 22 bp, artificial sequence
  synthetic
gaagggaaatagcaaatggaca

SEQ ID NO: 49 - DNA, 20 bp, artificial sequence
  synthetic
cgatggagtccaagttctgg

SEQ ID NO: 50 - DNA, 20 bp, artificial sequence
  synthetic
agcacttacagctctggcca

SEQ ID NO: 51 - DNA, 22 bp, artificial sequence
  synthetic
gactgaacataactgtaggctg

SEQ ID NO: 52 - DNA, 20 bp, artificial sequence
  synthetic
gttcggcacggtgtataagg

SEQ ID NO: 53 - DNA, 20 bp, artificial sequence
  synthetic
ctggccatcacgtaggcttc